(19) United States
(12) Patent Application Publication
(10) Pub. No.: US 2021/0379078 A1
(43) Pub. Date: Dec. 9, 2021

(54) Killing Senescent Cells And Treating Senescence-Associated Conditions Using A Bcl Inhibitor And An Mcl-1 Inhibitor

(71) Applicant: Unity Biotechnology, Inc., South San Francisco, CA (US)

(72) Inventors: Pieter Bas Kwak, South San Francisco, CA (US); Scott Armstrong, South San Francisco, CA (US); Pedro Beltran, South San Francisco, CA (US); Anne-Marie Beausoleil, South San Francisco, CA (US)

(21) Appl. No.: 17/285,420

(22) PCT Filed: Oct. 24, 2019
(86) PCT No.: PCT/US2019/057821

§ 371 Date: Apr. 14, 2021

Related U.S. Application Data
(60) Provisional application No. 62/752,938, filed on Oct. 30, 2018.

(51) Int. Cl. A61K 31/553; A61K 31/4725; G01N 33/50; A61K 31/519; A61K 31/4162; A61P 11/00; A61P 19/02; A61P 27/02; A61P 9/10

(52) U.S. Cl. CPC A61K 31/553; A61K 31/4725; G01N 33/502; A61K 31/519; A61P 9/10; A61P 11/00; A61P 19/02; A61P 27/02; A61K 31/4162

(57) ABSTRACT

This invention is based on the discovery that inhibiting more than one pathway in senescent cells leading to apoptosis has a profound effect: namely, increasing the potency or the cell specificity of the therapy. Combining a Bcl inhibitor with an Mcl 1 inhibitor increases the ability of the Bcl inhibitor to remove senescent cells from the site of an adverse condition synergistically. This increases the types of senescent cells that can be targeted, broadens the therapeutic range, and allows the user to tailor a particular combination of agents by adjusting the molar ratio for the patient being treated. Suitable indications for treatment may include any condition thought to be mediated at least in part by senescent cells, such as ophthalmic conditions, pulmonary conditions, and atherosclerosis.

### CROSS-REFERENCE TO RELATED APPLICATIONS

This application claims the benefit of U.S. Provisional Application No. 62/752,938, filed Oct. 30, 2018, the disclosure of which is incorporated herein by reference in its entirety.

### FIELD OF THE INVENTION

The technology disclosed and claimed below relates generally to the field of senescent cells and their role in age-related conditions. In particular, this disclosure provides for combination senolytic therapies useful for treating senescence-associated diseases or disorders.

### INCORPORATION BY REFERENCE

The following patents and patent applications are hereby incorporated herein by reference in their entirety for all purposes, including the removal of senescent cells, the treatment of senescence-related diseases in general, and the treatment of particular conditions such as atherosclerosis, eye disease, lung disease, liver disease, and atherosclerosis: U.S. Pat. No. 10,010,546; pre-grant publications US 2017/0266211 A1, and US 2018/0000816 A1; International Application Nos. PCT/US2018/046553 and PCT/US2018/046567; and U.S. provisional application 62/682,655.

The following patents, patent applications and scientific publications are hereby incorporated herein by reference in their entirety for all purposes, including the synthesis, formulation and use of Bcl inhibitors: U.S. Pat. Nos. 8,691,184; 9,096,625, and 10,010,546; pre-grant publication US 2017/0281649 A1; international application PCT/US2018/046553; and U.S. provisional applications 62/664,850; 62/664,891; 62/664,860; 62/664,863, 62/684,681; PCT publications WO 2017/101851, WO 2018/033128, WO 2018/052120, WO 2006/050447; Cancer Cell, 2016, 30(6), 834-835, doi:10.1016/j.ccell.2016.11.016; Nat. Struct. Mol. Biol., 2016, 23(6), 600-607, doi:10.1038/nsmb.3223; FEBS Lett., 2016, 591(1), 240-251, doi:10.1002/1873-3468.12497; European Journal of Cancer, 50, 6, 109-110, doi:10.1016/50959-8049(14)70464-2; J. Med. Chem., 2008, 51, 717-720, doi:10.1021/jm701358v; J. Med. Chem., 2007, 50, 8, 1723-1726, doi:10.1021/jm0614001; J. Med. Chem., 2013, 56, 3048-3067, doi:10.1021/jm4001105; J. Med. Chem., 2013, 56, 3048-3067, doi:10.1021/jm4001105; Blood, 2015, 126, 363-372, doi:10.1182/blood-2014-10-604975; Cancer Discovery, 2018, Ramsey et al., doi:10.1158/2159-8290.CD-18-0140; Cancer Res., 2013, 73(17), 5485-96, doi:10.1158/0008-5472.CAN-12-2272; and Expert Opin. Ther. Targets, 2013, 17(1), 61-75, doi:10.1517/14728222.2013.733001.

The following PCT Publications are hereby incorporated herein by reference in their entirety for all purposes, including the synthesis, formulation and use of Bcl-xL inhibitors: WO 2018/033128, WO 2018/092064, WO 2016/094517, WO 2016/094509, WO 2016/094505, WO 2012/103059, WO 2006/069186.

The following patents, patent applications and scientific publications are hereby incorporated herein by reference in their entirety for all purposes, including the synthesis, formulation and use of Mcl-1 inhibitors: U.S. Pat. Nos. 9,562,061; 9,840,518; PCT Publications: WO 2017/125224, WO 2018/144680, WO 2007/008627, WO 2008/130970, WO 2008/131000, WO 2014/047427, WO 2015/5031608, WO 2015/148854, WO 2013/052943, WO 2013/149124, WO 2015/153959, WO 2011/094708, WO 2013/142281, WO 2012/122370, WO 2010/024783, WO 2015/097123, WO 2016/033486, WO 2018/183418, WO 2018/178227, WO 2018/178226, WO 2018/127575, WO 2018/015526, WO 2017/182625, WO 2017/152076, WO 2017/147410, WO 2017/011323, WO 2016/200726; scientific publications: Cell Death Dis., 2015, 6, e1590, doi:10.1038/cddis.2014.561; Mol. Cancer Ther., 2014, 13(3), 565-575, doi:10.1158/1535-7163.MCT-12-0767; Cancer Research, 2017, Kump et al., doi:10.1158/1538-7445.AM2017-1173; FEBS Lett., 2017, 591(1), 240-251, doi:10.1002/1873-3468.12497; Cancer Res., 2006, 66(17), 8698-8706, Zeitlin et al.

For all purposes in the United States and in other jurisdictions where effective, each and every publication and patent document cited in this disclosure is hereby incorporated herein by reference in its entirety for all purposes to the same extent as if each such publication or document was specifically and individually indicated to be incorporated herein by reference.

### BACKGROUND

Some of the research conducted recently is focused on the premise that cells that have lost replicative capacity (known as senescent cells) remain in the tissue, where they trigger, mediate, or exacerbate age-related conditions. The senescent cells are thought to produce a constellation of secreted factors that act as cytokines, pro-inflammatory agents, and other compounds that cause degree progression and adverse symptoms, such as pain.

Local clearance of senescent cells attenuates the development of post-traumatic osteoarthritis and creates a pro-regenerative environment. O. H. Jeon et al., Nat. Med. 23(6):775, 2017. Small-molecule drugs have been identified that selectively remove senescent cells in and around the affected area, potentially alleviating adverse signs and symptoms of the condition, leaving other cells intact. Several intracellular pathways that are active in senescent cells can be targeted: for example, the Bcl-2 and Bcl-xL pathways (US 2018/117173 A1: Krizhanovsky et al., Yeda; US 2017/0056421 A1: Zhou et al., Arkansas; U.S. Pat. No. 9,849,128: Laberge et al.), the MDM2 pathway (U.S. Pat. No. 9,849,128: Laberge et al.), and the FLIP pathway (US 2018/021323 A1).

The technology described in this patent application represents a further advance in the development of senolytic agents for eliminating senescent cells and resolving age-related conditions.

### SUMMARY

This invention is based on the discovery that inhibiting more than one pathway in senescent cells leading to apoptosis has a profound effect: specifically to increase the potency or the cell specificity of the therapy. Combining a Bcl inhibitor with an Mcl-1 inhibitor in accordance with this invention increases the ability of the Bcl inhibitor to remove senescent cells from the site of an adverse condition—not just additively, but synergistically. Some Bcl and Mcl-1 inhibitors that are ineffective on their own when used in vivo may be combined to form a potent duo that is effective for treatment of a wide range of conditions that are thought to be mediated by senescent cells.

The technology provided in this disclosure represents an important advance in the science of senolytic medicine in several ways. First, effective combinations of the two agents has the ability to eliminate senescent cells in particular tissues that may not be easily amenable to treatment via a single senolytic agent. Second, even where single agents are effective for eliminating target cells, the synergistic effect of Bcl Mcl-1 combinations means that the tissue burden of the combined therapy (in terms of molecular mass) is substantially reduced. This has the potential benefit of increasing the therapeutic range for a particular target, increasing the potency against target cells while decreasing the risk of side effects. Third, the ability to adjust the molar ratio of the two agents allows the user to fine-tune the effect of the combination for a particular tissue target or a particular patient.

To our knowledge, this is the first demonstration that a combination of two different inhibitors targeting apoptosis can be used effectively to treat diseases mediated by senescent cells.

Combination senolytic therapies are described and exemplified herein comprising Bcl inhibitors and Mcl-1 inhibitors. Contacting senescent cells in vitro or in vivo with the senolytic combinations of the invention selectively eliminates such cells. The inhibitors can be used for administration to a target tissue in a subject having an age-related senescence-associated disease or disorder, thereby selectively eliminating senescent cells in or around the tissue and relieving one or more symptoms or signs of the conditions.

Specifically contemplated inventive embodiments are as follows:

A method for treating a senescence-associated disease or disorder comprising administering to a subject in need thereof therapeutically-effective amounts of a Bcl inhibitor and an Mcl-1 inhibitor.

The method of embodiment 1, wherein said Bcl inhibitor and Mcl-1 inhibitor selectively kill senescent cells.

A method for selectively killing a senescent cell, comprising contacting the cell with an effective amount of a senolytic combination, wherein the senolytic combination is a means for inhibiting Bcl and a means for inhibiting Mcl-1.

A method of enhancing the senolytic activity of a Bcl inhibitor and/or the therapeutic efficacy of the Bcl inhibitor for treating a senescence associated disease or disorder, wherein the method comprises combining the Bcl inhibitor with a means for inhibiting Mcl-1.

A method of enhancing the senolytic activity of an Mcl-1 inhibitor and/or the therapeutic efficacy of the Mcl-1 inhibitor for treating a senescence associated disease or disorder, wherein the method comprises combining the Mcl-1 inhibitor with a means for inhibiting Bcl.

The method of embodiments 1-5, wherein the senescence-associated disease or disorder is not cancer.

The method of embodiments 1-6, wherein the Bcl inhibitor is a Bcl-2/Bcl-xL/Bcl-w inhibitor, a Bcl-2/Bcl-xL inhibitor, a Bcl-xL/Bcl-w inhibitor, or a Bcl-xL selective inhibitor.

The method of embodiments 1-7, wherein the Bcl inhibitor is any one of the Bcl inhibitors listed or exemplified in this disclosure.

The method of embodiments 1-8, wherein the Mcl-1 inhibitor is a small molecule compound, a peptide mimetic, a BH3-derived peptide, or a stapled peptide.

The method of embodiments 1-9, wherein the Mcl-1 inhibitor is any one of the Mcl-1 inhibitors listed or exemplified in this disclosure.

The method of embodiments 1-10, wherein the Bcl inhibitor is navitoclax (ABT263) and the Mcl-1 inhibitor is selected from AMG-176, AZD-5991, S-63845, and A1210477.

The method of embodiments 1-10, wherein the Bcl inhibitor is (R)-5-(4-chlorophenyl)-4-(3-fluoro-5-(4-(4-(4-(4-(4-(hydroxymethyl)piperidin-1-yl)-1-(phenylthio)butan-2-ylamino)-3-(trifluoromethylsulfonyl)phenylsulfonamido)phenyl)piperazin-1-yl)phenyl)-1-isopropyl-2-methyl-1H-pyrrole-3-carboxylic acid (Compound 26) and the Mcl-1 inhibitor is selected from AMG-176, AZD-5991, and S-63845.

The method of embodiments 1-10, wherein the Bcl inhibitor is A1331852 and the Mcl-1 inhibitor is AMG-176.

The method of embodiments 1-13, wherein the Bcl inhibitor and the Mcl-1 inhibitor in combination have a synergy coefficient (δ) greater than 10 for killing irradiated small airway epithelial cells (SAEC).

The method of embodiments 14, wherein the synergy coefficient (δ) is between 10-100.

The method of embodiments 1-15, wherein the senescent cells are senescent endothelial cells, senescent fibroblasts, senescent mesenchymal cells, senescent chondrocytes, or senescent synoviocytes.

The method of embodiments 1-15, wherein the cells are senescent epithelial cells.

The method of embodiments 1-16, wherein the senescence-associated disease or disorder is atherosclerosis.

The method of embodiments 1-16, wherein the senescence-associated disease or disorder is osteoarthritis.

The method of embodiments 1-16, wherein the senescence-associated disease or disorder is a pulmonary disease, such as idiopathic pulmonary fibrosis (IPF) or chronic obstructive pulmonary disease (COPD).

The method of embodiments 1-16, wherein the senescence-associated disease or disorder is an eye disease or disorder, such as age-related macular degeneration, glaucoma, or diabetic retinopathy.

The method of claims 1-16, wherein the senescence-associated disease or disorder is a liver disease, such as non-alcoholic steatohepatitis (NASH), primary biliary cholangitis (PBC), or primary sclerosing cholangitis (PSC).

The method of embodiments 1-2, 4-22, wherein the Bcl inhibitor and the Mcl-1 inhibitor are administered as a combination within at least one treatment cycle, which treatment cycle comprises a treatment course followed by a non-treatment interval; and wherein the total dose of the combination administered during the treatment cycle is an amount less than the amount effective for a cancer treatment.

The method of embodiment 3, wherein the senolytic combination contacts the senescent cell within at least one treatment cycle, which treatment cycle comprises a treatment course followed by a non-treatment interval; and wherein the total dose of the senolytic combination administered during the treatment cycle is an amount less than the amount effective for a cancer treatment.

The method of embodiments 1-17, 19-21, 23, wherein the Bcl inhibitor and the Mcl-1 inhibitor are administered directly to an organ or tissue affected by the senescence-associated disease or disorder that comprises the senescent cells.

The method of embodiments 18, 22-23, wherein the Bcl inhibitor and the Mcl-1 inhibitor are administered systemically.

The method of embodiment 2 or 22, wherein the senolytic combination is administered directly to an organ or tissue affected by the senescence-associated disease or disorder that comprises the senescent cells.

The method of embodiments 3, 18, 22, and 24, wherein the senolytic combination is administered systemically.

A combination of a Bcl inhibitor medicament and an Mcl-1 inhibitor medicament for treating a senescence-associated disease or disorder, wherein the Bcl inhibitor medicament and the Mcl-1 inhibitor medicament selectively kill senescent cells.

Use of a Bcl inhibitor in combination with an Mcl-1 inhibitor for the manufacture of a medicament for the treatment of a senescence-associated disease or disorder in a subject.

A unit dose of a pharmaceutical composition that is formulated for relief of symptoms of a senescence-associated disease or disorder at a disease site in a subject in need thereof;

wherein the unit dose contains an amount of a first compound that constitutes a means for selectively inhibiting Bcl and an amount of a second compound that constitutes a means for specifically inhibiting Mcl-1 in a formulation that is configured for administration in or around the site of the disorder in the subject the subject;

wherein the formulation of the composition, the amount of the first compound, the amount of the second compound, and the molar ratio of the first compound to the second compound configure the unit dose such that one or more administrations of the unit dose in or around the disease site during a treatment period is effective in selectively removing senescent cells from the disease site, and

thereby providing the subject with a subsequent therapeutic period during which the signs or symptoms of the senescence-associated disease or disorder are relieved as a result of the administration of the composition to the disease site during the treatment period.

A method of identifying a combination of medicaments that is effective for killing senescent cells or treating a senescence-associated disease or disorder, the method comprising:

(1) contacting a senescent cell (such as an irradiated cell) with a predetermined concentration and molar ratio of a test Bcl inhibitor and a test Mcl-1 inhibitor;

(2) contacting a non-senescent cell (such as a non-irradiated cell of the same tissue type) with the same concentration and molar ratio of the test Bcl inhibitor and the test Mcl-1 inhibitor; and

(3) identifying the test Bcl inhibitor and the test Mcl-1 inhibitor as an effective combination of medicaments at said concentration and molar ratio if the combination has an LD50 that is selective (such as 3, 5, or 10 times lower) for the senescent cells compared with the non-senescent cells.

The invention is set forth in the above embodiments, in the description that follows, in the figures, experimental examples, and in the appended claims.

### DRAWINGS

FIGS. 1A-1D demonstrate the ability to induce senescence in primary human epithelial cells by irradiation, where FIG. 1A demonstrates normal, non-senescent cells (NsC), as validated by the detection of senescence β-galactosidase staining (FIGS. 1B and 1C) and by qPCR detecting p16 (FIG. 1D). See Example 1.

FIGS. 2A-2C demonstrate a concentration-response curve for the senolytic combination of navitoclax and AMG-176 (FIG. 2A, 2B), as compared to navitoclax alone in FIG. 2C, which demonstrates sensitivity of senescent lung epithelial cell survival (SnC) to incubation with a senolytic, whereas this senolytic combination shows limited senolysis in non-senescent cells (NsC). See Example 5.

FIGS. 3A-3C demonstrate that in both OA-dosed mouse lungs and MBE cells (FIG. 3A-3C), when BIM-Bcl-xL interactions were blocked by the aryl sulfonamide Bcl-2/Bcl-xL inhibitor Compound 1 (FIG. 3B), a compensatory Mcl-1 binding to BIM was observed (FIG. 3B). In FIG. 3C, Bcl-xL appeared to compensate for BIM binding when Mcl-1 was inhibited by the Mcl-1 inhibitor S-63845 at 1 μM and 10 μM. See Example 6.

FIGS. 4A-4D demonstrate dose-responses for cell viability on primary human SAECs, expressed as an EC50 for various senolytic combinations using the Bcl-2/Bcl-xL inhibitor navitoclax in combination with four different Mcl-1 inhibitors tested: AMG-176 (FIG. 4A), S-63845 (FIG. 4B), AZD-5991 (FIG. 4C), and A-1210477 (FIG. 4D). See Example 7.

FIGS. 5A-5D show heat maps of various dose-responses on primary human SAECs, expressed as a senolytic coefficient (δ), for various senolytic combinations using the Bcl-2/Bcl-xL inhibitor navitoclax in combination with four different Mcl-1 inhibitors tested: AMG-176 (FIG. 5A), S-63845 (FIG. 5B), AZD-5991 (FIG. 5C), and A-1210477 (FIG. 5D). See Example 7.

FIGS. 6A-6C demonstrates synergistic senolysis as measured by dose-responses for cell viability on primary human SAECs, expressed as an EC50 with an aryl sulfonamide Bcl-2/Bcl-xL inhibitor Compound 26 in combination with three different Mcl-1 inhibitors: AMG-176 (FIG. 6A), S-63845 (FIG. 6B), and AZD-5991 (FIG. 6C). See Example 7.

FIGS. 7A-7C show heat maps of various dose-responses on primary human SAECs, expressed as a senolytic coefficient (δ), demonstrates synergistic senolysis with an aryl sulfonamide Bch 2/Bcl-xL inhibitor Compound 26 in combination with three different Mcl-1 inhibitors: AMG-176 (FIG. 7A), S-63845 (FIG. 7B), and AZD-5991 (FIG. 7C). See Example 7.

FIGS. 8A-8B demonstrate synergistic senolysis as measured by dose-responses for cell viability on primary human SAECs, expressed as an EC50 with a Bcl-xL selective inhibitor A-1331852 in combination with the Mcl-1 inhibitor AMG-176 (FIG. 8A), and Venetoclax, a known Bcl-2-selective inhibitor, in combination with the Mcl-1 inhibitor AMG-176 (FIG. 8B). See Example 7.

FIGS. 9A-9B shows heat maps of dose-responses on primary human SAECs, expressed as a senolytic coefficient (δ) using a Bcl-xL selective inhibitor A-1331852 in combination with the Mcl-1 inhibitor AMG-176 (FIG. 9A) and Venetoclax, a known Bcl-2-selective inhibitor, in combination with the Mcl-1 inhibitor AMG-176 (FIG. 9B). See Example 7.

FIG. 10 shows the effect of Compound 1+AZD-5991 on bleomycin-induced p16 expression in mouse lung epithelial cells at different concentrations of AZD-5991(0.3 mg/ml, 0.5 mg/ml, 1 mg/ml). See Example 8.

FIG. 11 shows the effect of Compound 1+AZD-5991 on caspase 3/7 activity in bleomycin-induced mouse lung epithelial cells at different concentrations of AZD-5991(0.3 mg/ml, 0.5 mg/ml, 1 mg/ml). See Example 8.

### DETAILED DESCRIPTION

Senescent cells are characterized as cells that no longer have replicative capacity, but remain in the tissue of origin, eliciting a senescence-associated secretory phenotype (SASP). Senescent cells accumulate with age, which is why disease conditions mediated by senescent cells occur more frequently in older adults. It is a premise of this disclosure that many age-related disease conditions are mediated by senescent cells, and that selective removal of the cells from tissues at or around the disease condition can be used clinically for the treatment of such conditions.

The technology described and claimed below describes combination senolytic therapies that can be used to selectively eliminate senescent cells from a target tissue for purposes of treatment of senescence-associated diseases or disorders.

### Inhibition of Bcl Protein Activity

The Bcl protein family (TC #1.A.21) includes evolutionarily-conserved proteins that share Bcl-2 homology (BH) domains. Bcl proteins are most notable for their ability to up- or down-regulate apoptosis, a form of programmed cell death, at the mitochondrion. The following explanation is provided to assist the user in understanding some of the scientific underpinnings of the methods and senolytic combinations of the invention. These concepts are not needed to practice the invention, nor do they limit the use of the senolytic combinations and methods described herein in any manner beyond that which is expressly stated or required.

In the context of this invention, the Bcl proteins of particular interest are those that downregulate apoptosis. Anti-apoptotic Bcl proteins contain BH1 and BH2 domains, some of them contain an additional N-terminal BH4 domain (Bcl-2, Bcl-xL and Bcl-w (Bcl-2L2), inhibiting these proteins increases the rate or susceptibility of cells to apoptosis. Thus, an inhibitor of such proteins can be used to help eliminate cells in which the proteins are expressed.

In the mid-2000s, Abbott Laboratories developed a novel inhibitor of Bcl-2, Bcl-xL and Bcl-w, known as ABT-737 (Navitoclax) as an oncology therapeutic. This compound is part of a group of BH3 mimetic small molecule inhibitors that target these Bcl-2 family proteins, but not Al or Mcl-1. ABT-737 was superior to previous Bcl-2 inhibitors given its higher affinity for Bcl-2, Bcl-xL and Bcl-w. In vitro studies showed that primary cells from patients with B-cell malignancies are sensitive to ABT-737. In human patients, ABT-737 is effective against some types of cancer cells, but is subject to dose-limiting thrombocytopenia.

### Bcl Inhibitors

This section provides compounds that constitute a means for inhibiting members of the Bcl family, particularly Bcl-2, Bcl-xL, Bcl-w, and combinations thereof. They are suitable for testing as senolytic agents in combination with Mcl-1 inhibitors, according to this invention.

One class of exemplary Bcl inhibitors includes, for example, A-107250, A-1155463, A-1331852, AB141523 (2-methoxy-antimycin A3), ABT-737, APG-2575, APG-1252 (BM-1252), APG-2575, AZ-Mcl1, BH3I-1, (−)BI97D6, BM-903, BM-956, BM-957, BM-1074, BM-1197, BXI-61 (NSC354961, 3-[(9-Amino-7-ethoxyacridin-3-yl)diazenyl]pyridine-2,6-diamine) or BXI-72 (NSC334072, bisbenzimide), 2,3-DCPE (2-[[3-(2,3-Dichlorophenoxy)propyl]amino]ethanol), EU5346 (ML311), gossypols, gossypol (BL 193), (−)-gossypol ((−)BL 193), (+)-gossypol ((+)BL 193), R-(−)-gossypol (AT-101), S-(−)-gossypol, apogossypol, gossypolone, HA14-1, JY-1-106, MAIM1, Navitoclax (ABT-263), Obatoclax (GX15-070) pyrogallols, acylpyrogallols, 563845, Sabutoclax (BI-97C1), TM-179, TM-1206, Venetoclax (ABT-199, GDC-0199, RG7601), UM-36, VU661013, WEHI-539, ((R) 4-(4-chlorophenyl)-3-(3-(4-(4-(4-((4-(dimethylamino)-1-(phenylthio)butan-2-yl)amino)-3-nitrophenylsulfonamido)phenyl)piperazin-1-yl)phenyl)-5-ethyl-1-methyl-1H-pyrrole-2-carboxylic acid (“Compound 21”), (R)-5-(4-chlorophenyl)-4-(3-(4-(4-(4-((4-(dimethylamino)-1-(phenylthio)butan-2-yl)amino)-3-nitrophenylsulfonamido)phenyl)piperazin-1-yl)phenyl)-1-ethyl-2-methyl-1H-pyrrole-3-carboxylic acid (“Compound 14”), (R)-5-(4-chlorophenyl)-4-(3-(4-(4-(4-(4-(dimethylamino)-1-(phenylthio)butan-2-ylamino)-3-nitrophenylsulfonamido)phenyl)piperazin-1-yl)phenyl)-1-isopropyl-2-methyl-1H-pyrrole-3-carboxylic acid (“Compound 15”), and pharmaceutically acceptable salts thereof.

Included in the class of exemplary Bcl inhibitors are aryl sulfonamides that have the following structure:

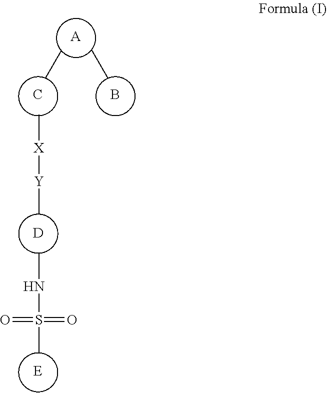

wherein A is an optionally substituted 2, 3-1H-pyrrolylene; B and E individually are optionally substituted phenyl; C is optionally substituted 1,3-phenylene; D is optionally substituted 1,4-phenylene; and X and Y taken together form the following:

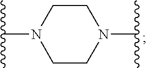 [SMILES: *N1CCN(*)CC1]

Other exemplary aryl sulfonamides Bcl inhibitors include those that have the following structures:

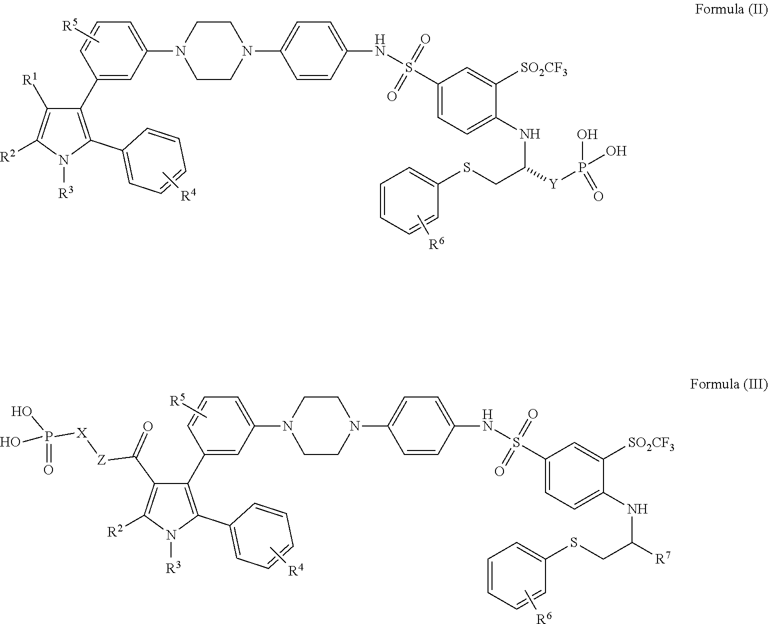

wherein X is substituted or unsubstituted, is selected from the group consisting of alkylene, alkenylene, cycloalkylene, cycloalkenylene, and heterocycloalkylene; Y is selected from the group consisting of (CH₂)ₙ—N(R^{a}) and:

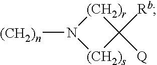

Q is selected from the group consisting of O, O(CH₂)₁₋₃, NR^{c}, NR^{c}(C₁₋₃alkylene), OC(═O)(C₁₋₃alkylene), C(═O)O, C(═O)O(C₁₋₃alkylene), NHC(═O)(C₁₋₃alkylene), C(═O)NH, and C(═O)NH(C₁₋₃alkylene);
Z is O or NR^{c};

R¹ and R², independently, are selected from the group consisting of H, CN, NO₂, halo, alkyl, cycloalkyl, alkenyl, cycloalkenyl, alkynyl, aryl, heteroaryl, heterocycloalkyl, OR′, SR′, NR′R″, COR′, CO₂R′, OCOR′, CONR′R″, CONR′SO₂R″, NR′COR″, NR′CONR″R′″, NR′C═SNR″R′″, NR′SO₂R″, SO₂R′, and SO₂NR′R″;

R³ is selected from a group consisting of H, alkyl, cycloalkyl, alkenyl, cycloalkenyl, alkynyl, aryl, heteroaryl, heterocycloalkyl, OR′, NR′R″, OCOR′, CO₂R′, COR′, CONR′R″, CONR′SO₂R″, C₁₋₃alkyleneCH(OH)CH₂OH, SO₂R′, and SO₂NR′R″;

R′, R″, and R′″, independently, are H, alkyl, cycloalkyl, alkenyl, cycloalkenyl, alkynyl, aryl, heteroaryl, C₁₋₃alkyleneheterocycloalkyl, or heterocycloalkyl;

R′ and R″, or R″ and R′″, can be taken together with the atom to which they are bound to form a 3 to 7 membered ring;

R⁴ is hydrogen, halo, C₁₋₃alkyl, CF₃, or CN;

R⁵ is hydrogen, halo, C₁₋₃alkyl, substituted C₁₋₃alkyl, hydroxyalkyl, alkoxy, or substituted alkoxy;

R⁶ is selected from the group consisting of H, CN, NO₂, halo, alkyl, cycloalkyl, alkenyl, cycloalkenyl, alkynyl, aryl, heteroaryl, heterocycloalkyl, OR′, SR′, NR′R″, CO₂R′, OCOR′, CONR′R″, CONR′SO₂R″, NR′COR″, NR′CONR″R′″, NR′C═SNR″R′″, NR′SO₂R″, SO₂R′, and SO₂NR′R″;

R⁷, substituted or unsubstituted, is selected from the group consisting of hydrogen, alkyl, alkenyl, (CH₂)₀₋₃cycloalkyl, (CH₂)₀₋₃cycloalkenyl, (CH₂)₀₋₃heterocycloalkyl, (CH₂)₀₋₃aryl, and (CH₂)₀₋₃ heteroaryl;

R⁸ is selected from the group consisting of hydrogen, halo, NO₂, CN, SO₂CF₃, and CF₃;

R^{a} is selected from the group consisting of hydrogen, alkyl, heteroalkyl, alkenyl, hydroxyalkyl, alkoxy, substituted alkoxy, cycloalkyl, cycloalkenyl, and heterocycloalkyl;

R^{b} is hydrogen or alkyl;

R^{c} is selected from the group consisting of hydrogen, alkyl, substituted alkyl, hydroxyalkyl, alkoxy, and substituted alkoxy;

and n, r, and s, independently, are 1, 2, 3, 4, 5, or 6.

Still other exemplary aryl sulfonamides Bcl inhibitors include those that have the following structure:

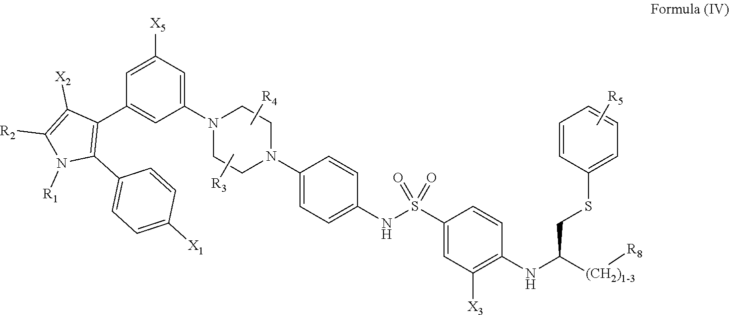

wherein:
R₁ and R₂ are independently C₁ to C₄ alkyl;
R₃, R₄ and R₅ are independently —H or —CH₃;
R₈ is —OH or —N(R₆)(R₇), wherein R₆ and R₇ are independently alkyl or heteroalkyl, and are optionally cyclized;
X₁ is —F, —Cl, —Br, or —OCH₃;
X₂ is —SO₂R′ or —CO₂R′, where R′ is —H, —CH₃, or —CH₂CH₃;
X₃ is —SO₂CF₃; —SO₂CH₃; or —NO₂
X₅ is —F, —Br, —Cl, —H, or —OCH₃.

More exemplary aryl sulfonamides Bcl inhibitors include those that have the following structure:

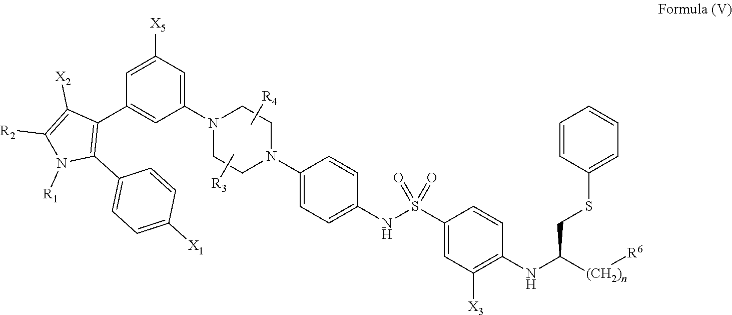

wherein:
X¹ is —Cl;
X² is —COOH or —SO₂CH₃;
X³ is —SO₂CF₃, —SO₂CH₃, or —NO₂;
X⁵ is —F or —H;
R¹ is —CH(CH₃)₂;
R² is —CH₃;
R³ and R⁴ are both —H;
n is 2;
   R⁶ is selected from —OH, —OR⁷,

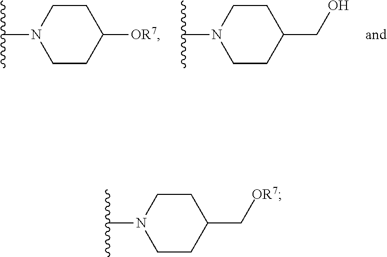

and
R⁷ is —PO(OH)₂, or a salt or a stereoisomer thereof.

Specifically, such exemplary aryl sulfonamide Bcl inhibitors include those in Table 1:

TABLE 1

| Compound | |
|---|---|
| No. | Compound Structure and Name |
| 1 | 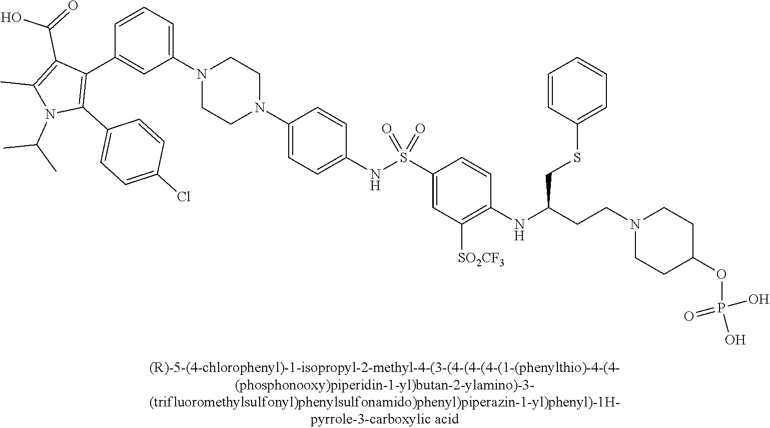 |
| 2 | 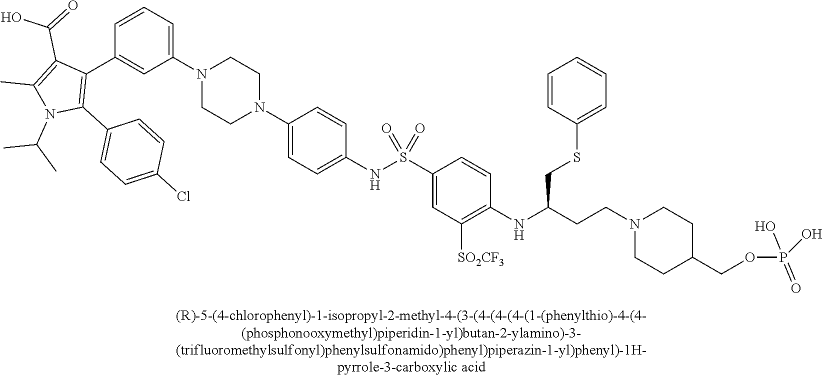 |
| 3 | 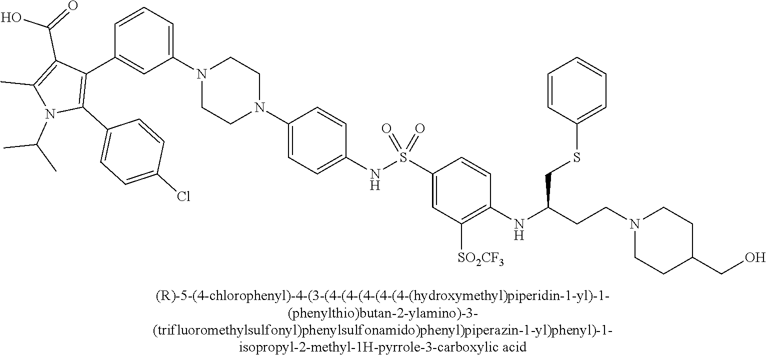 |
| 4 | 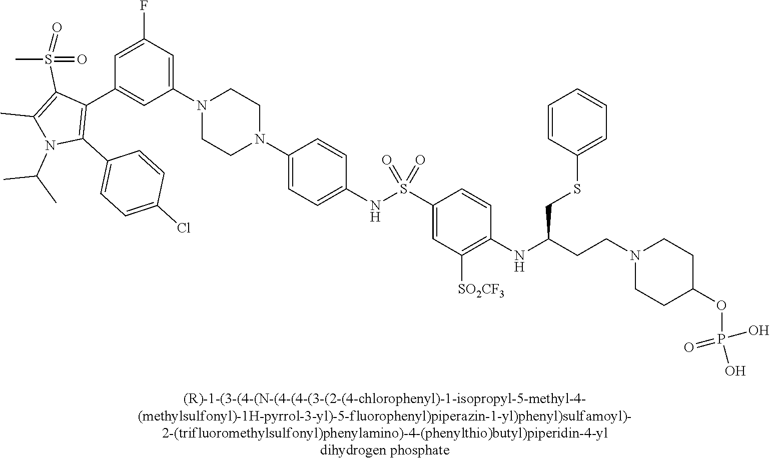 |
| 5 | 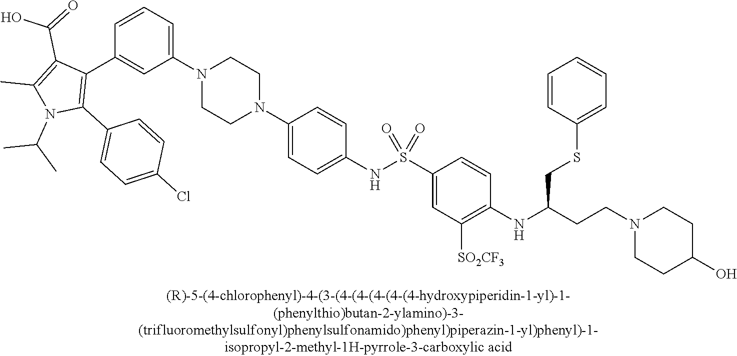 |
| 6 | 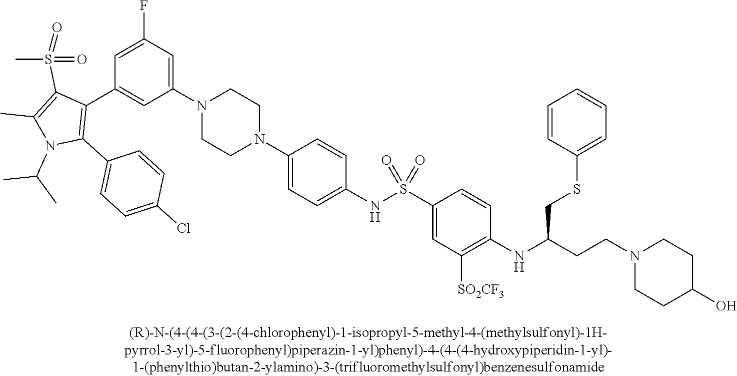 |
| 7 | 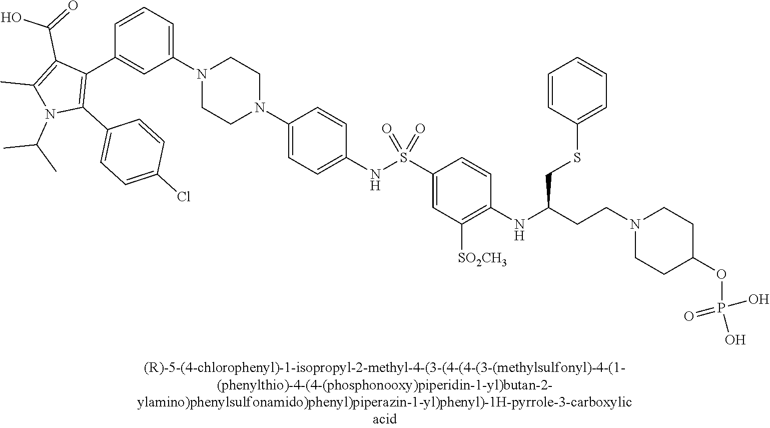 [SMILES: Cc1c(C(=O)O)c(-c2cccc(N3CCN(c4ccc(NS(=O)(=O)c5ccc(N[C@H](CCN6CCC(OP(=O)(O)O)CC6)CSc6ccccc6)c(S(C)(=O)=O)c5)cc4)CC3)c2)c(-c2ccc(Cl)cc2)n1C(C)C] |
| 8 | 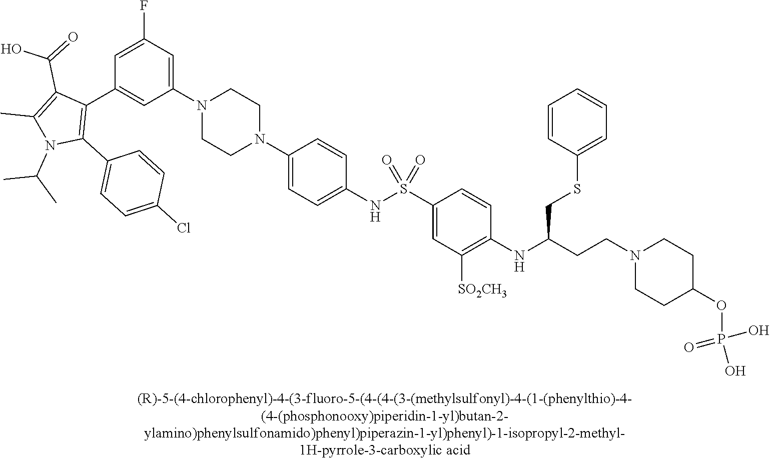 [SMILES: Cc1c(C(=O)O)c(-c2cc(F)cc(N3CCN(c4ccc(NS(=O)(=O)c5ccc(N[C@H](CCN6CCC(OP(=O)(O)O)CC6)CSc6ccccc6)c(S(C)(=O)=O)c5)cc4)CC3)c2)c(-c2ccc(Cl)cc2)n1C(C)C] |
| 9 | 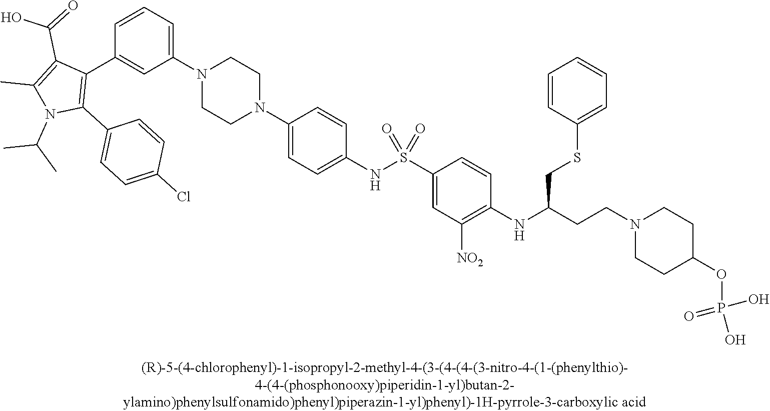 [SMILES: Cc1c(C(=O)O)c(-c2cccc(N3CCN(c4ccc(NS(=O)(=O)c5ccc(N[C@H](CCN6CCC(OP(=O)(O)O)CC6)CSc6ccccc6)c([N+](=O)[O-])c5)cc4)CC3)c2)c(-c2ccc(Cl)cc2)n1C(C)C] |
| 10 | 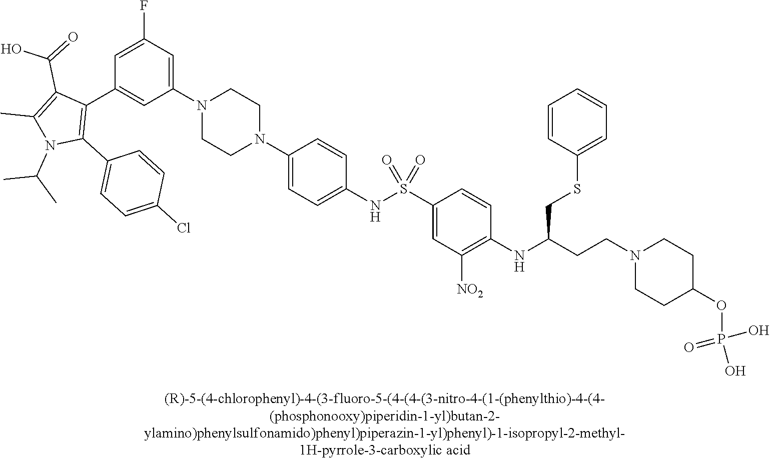 [SMILES: Cc1c(C(=O)O)c(-c2cc(F)cc(N3CCN(c4ccc(NS(=O)(=O)c5ccc(N[C@H](CCN6CCC(OP(=O)(O)O)CC6)CSc6ccccc6)c([N+](=O)[O-])c5)cc4)CC3)c2)c(-c2ccc(Cl)cc2)n1C(C)C] |
| 11 | 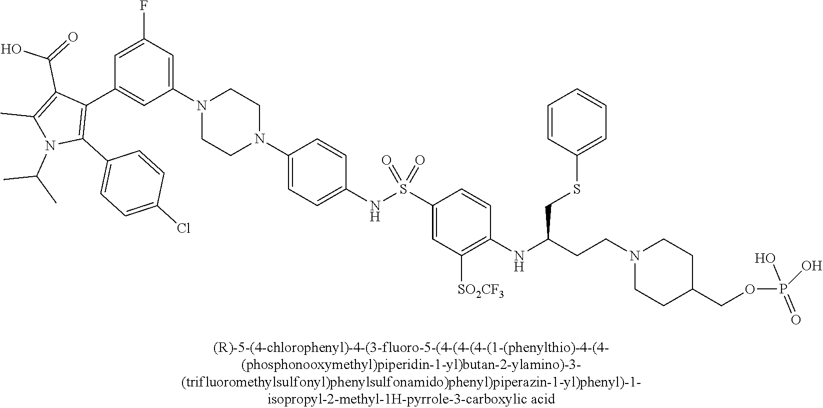 |
| 12 | 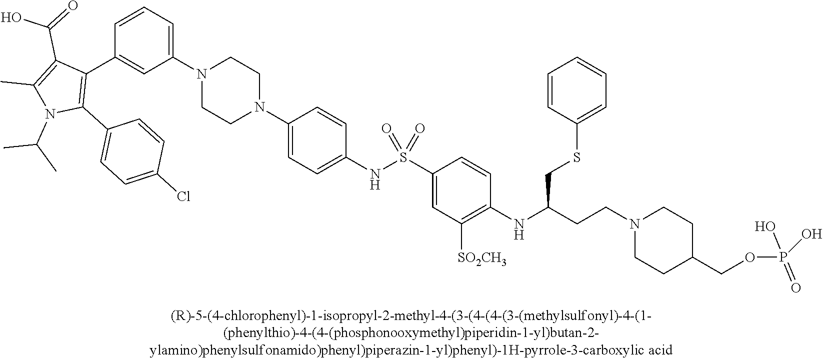 [SMILES: Cc1c(C(=O)O)c(-c2cccc(N3CCN(c4ccc(NS(=O)(=O)c5ccc(N[C@H](CCN6CCC(COP(=O)(O)O)CC6)CSc6ccccc6)c(S(C)(=O)=O)c5)cc4)CC3)c2)c(-c2ccc(Cl)cc2)n1C(C)C] |
| 13 | 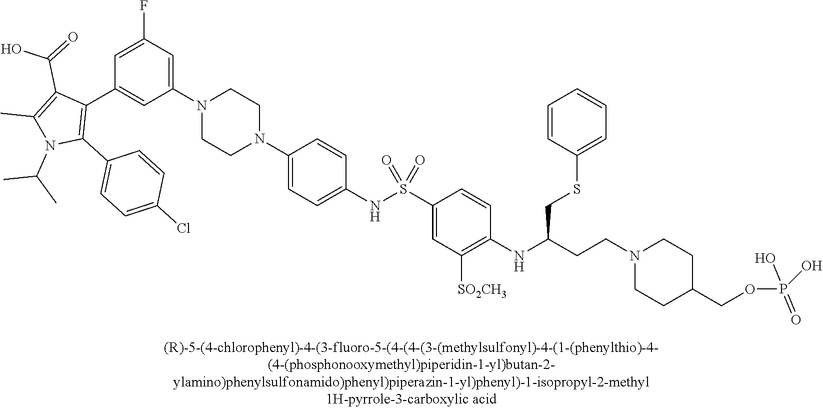 [SMILES: Cc1c(C(=O)O)c(-c2cc(F)cc(N3CCN(c4ccc(NS(=O)(=O)c5ccc(N[C@H](CCN6CCC(COP(=O)(O)O)CC6)CSc6ccccc6)c(S(C)(=O)=O)c5)cc4)CC3)c2)c(-c2ccc(Cl)cc2)n1C(C)C] |
| 14 | 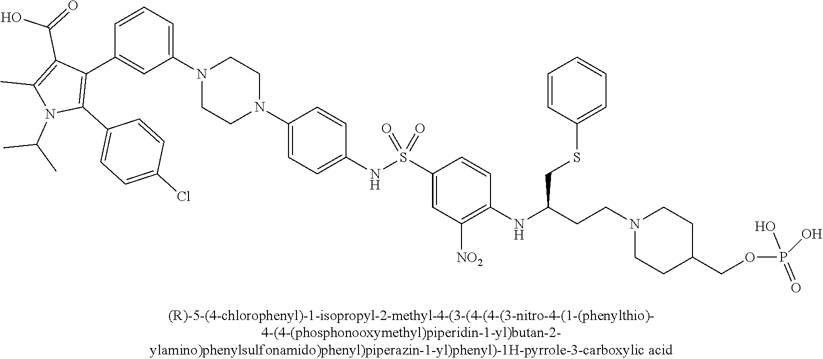 [SMILES: Cc1c(C(=O)O)c(-c2cccc(N3CCN(c4ccc(NS(=O)(=O)c5ccc(N[C@H](CCN6CCC(COP(=O)(O)O)CC6)CSc6ccccc6)c([N+](=O)[O-])c5)cc4)CC3)c2)c(-c2ccc(Cl)cc2)n1C(C)C] |
| 15 | 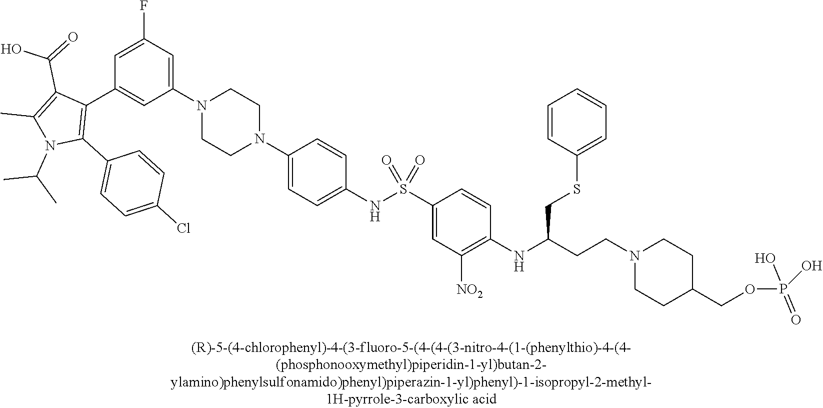 [SMILES: Cc1c(C(=O)O)c(-c2cc(F)cc(N3CCN(c4ccc(NS(=O)(=O)c5ccc(N[C@H](CCN6CCC(COP(=O)(O)O)CC6)CSc6ccccc6)c([N+](=O)[O-])c5)cc4)CC3)c2)c(-c2ccc(Cl)cc2)n1C(C)C] |
| 16 | 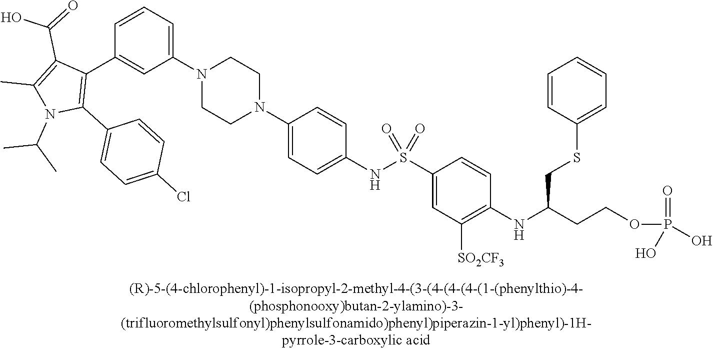 |
| 17 | 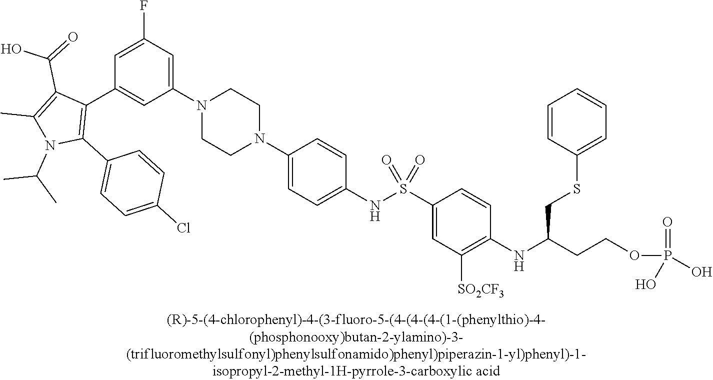 |
| 18 | 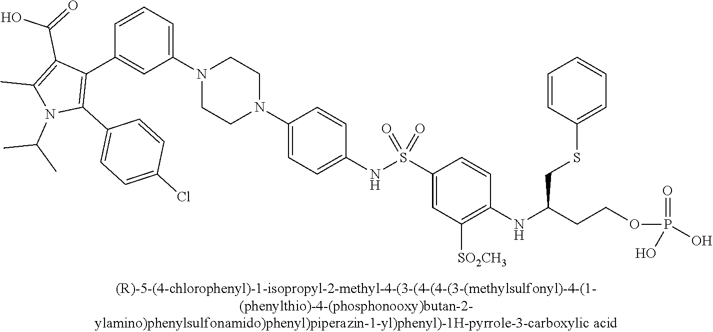 [SMILES: Cc1c(C(=O)O)c(-c2cccc(N3CCN(c4ccc(NS(=O)(=O)c5ccc(N[C@H](CCOP(=O)(O)O)CSc6ccccc6)c(S(C)(=O)=O)c5)cc4)CC3)c2)c(-c2ccc(Cl)cc2)n1C(C)C] |
| 19 | 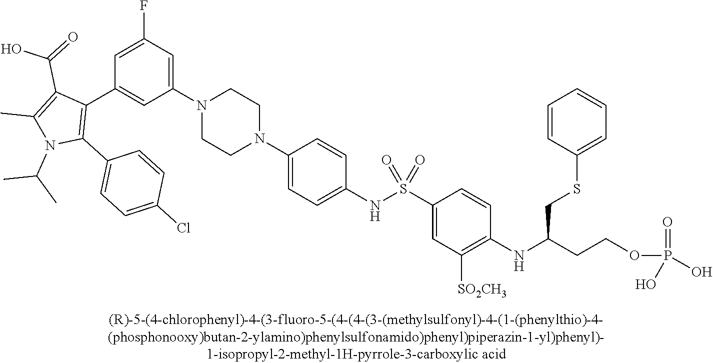 [SMILES: Cc1c(C(=O)O)c(-c2cc(F)cc(N3CCN(c4ccc(NS(=O)(=O)c5ccc(N[C@H](CCOP(=O)(O)O)CSc6ccccc6)c(S(C)(=O)=O)c5)cc4)CC3)c2)c(-c2ccc(Cl)cc2)n1C(C)C] |
| 20 | 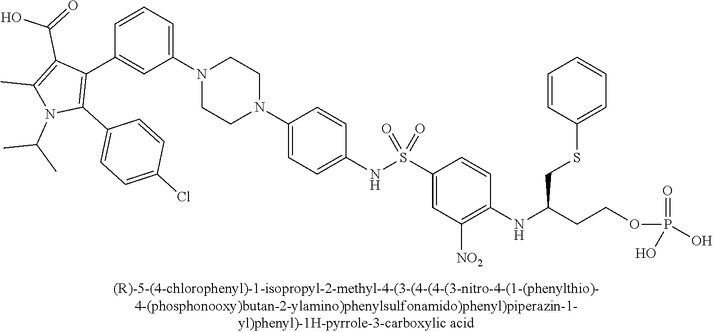 [SMILES: Cc1c(C(=O)O)c(-c2cccc(N3CCN(c4ccc(NS(=O)(=O)c5ccc(N[C@H](CCOP(=O)(O)O)CSc6ccccc6)c([N+](=O)[O-])c5)cc4)CC3)c2)c(-c2ccc(Cl)cc2)n1C(C)C] |
| 21 | 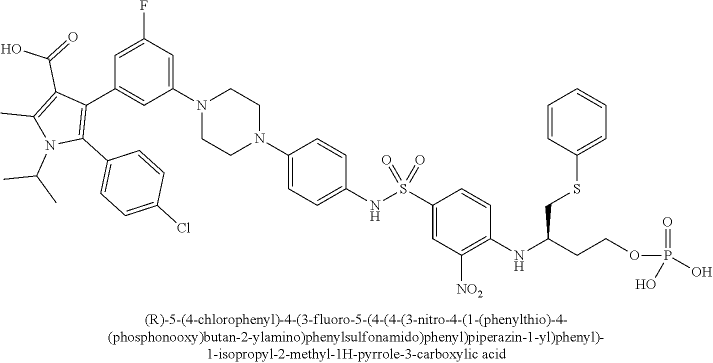 [SMILES: Cc1c(C(=O)O)c(-c2cc(F)cc(N3CCN(c4ccc(NS(=O)(=O)c5ccc(N[C@H](CCOP(=O)(O)O)CSc6ccccc6)c([N+](=O)[O-])c5)cc4)CC3)c2)c(-c2ccc(Cl)cc2)n1C(C)C] |
| 26 | 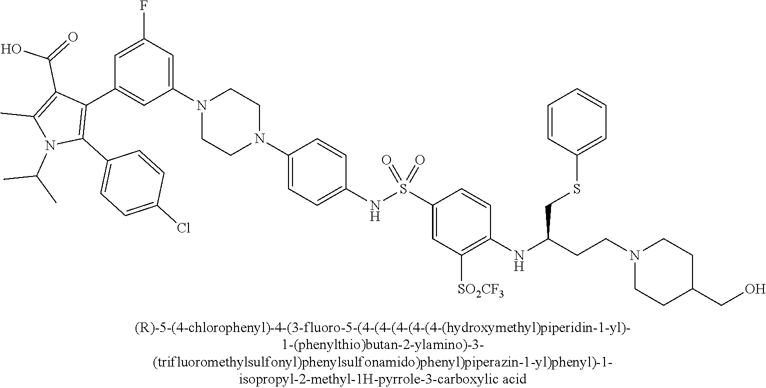 |
| 27 | 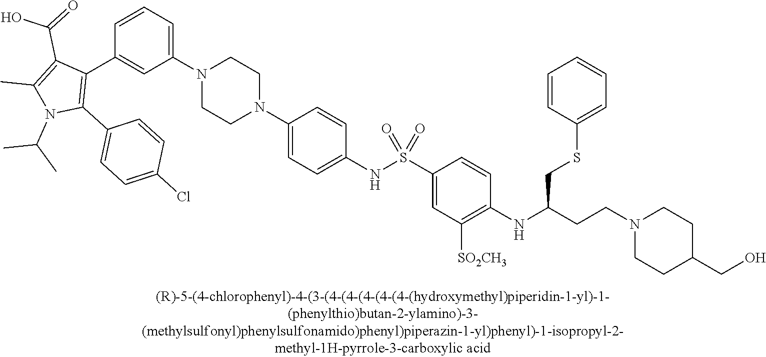 [SMILES: Cc1c(C(=O)O)c(-c2cccc(N3CCN(c4ccc(NS(=O)(=O)c5ccc(N[C@H](CCN6CCC(CO)CC6)CSc6ccccc6)c(S(C)(=O)=O)c5)cc4)CC3)c2)c(-c2ccc(Cl)cc2)n1C(C)C] |
| 28 | 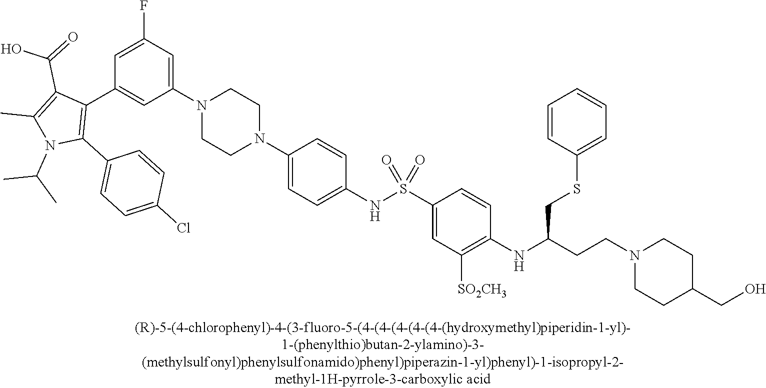 [SMILES: Cc1c(C(=O)O)c(-c2cc(F)cc(N3CCN(c4ccc(NS(=O)(=O)c5ccc(N[C@H](CCN6CCC(CO)CC6)CSc6ccccc6)c(S(C)(=O)=O)c5)cc4)CC3)c2)c(-c2ccc(Cl)cc2)n1C(C)C] |
| 29 | 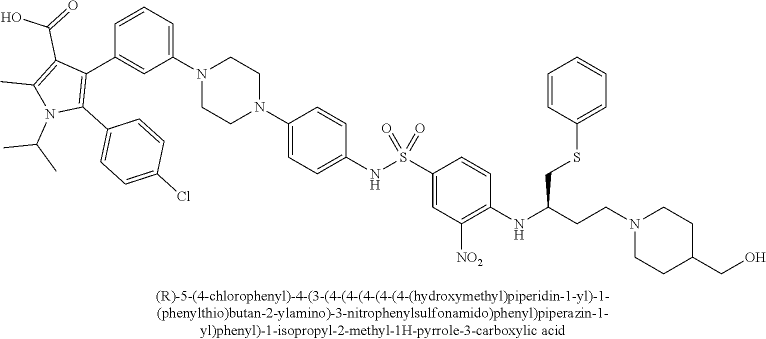 [SMILES: Cc1c(C(=O)O)c(-c2cccc(N3CCN(c4ccc(NS(=O)(=O)c5ccc(N[C@H](CCN6CCC(CO)CC6)CSc6ccccc6)c([N+](=O)[O-])c5)cc4)CC3)c2)c(-c2ccc(Cl)cc2)n1C(C)C] |
| 30 | 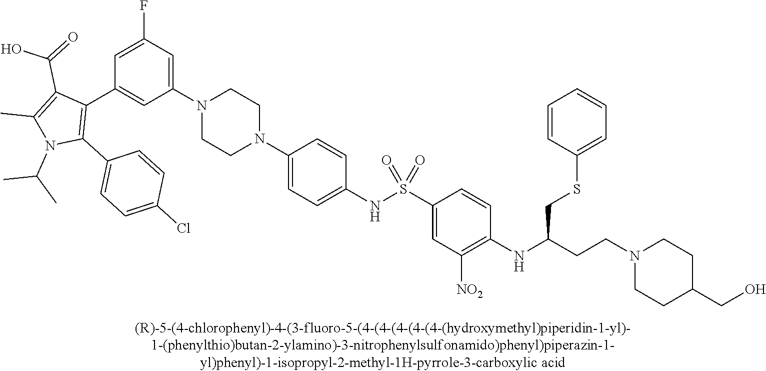 [SMILES: Cc1c(C(=O)O)c(-c2cc(F)cc(N3CCN(c4ccc(NS(=O)(=O)c5ccc(N[C@H](CCN6CCC(CO)CC6)CSc6ccccc6)c([N+](=O)[O-])c5)cc4)CC3)c2)c(-c2ccc(Cl)cc2)n1C(C)C] |
| 31 | 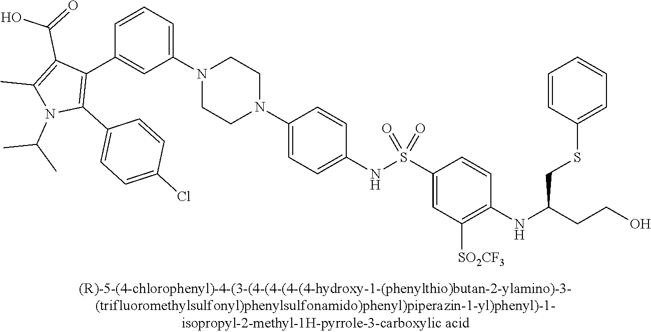 |
| 32 | 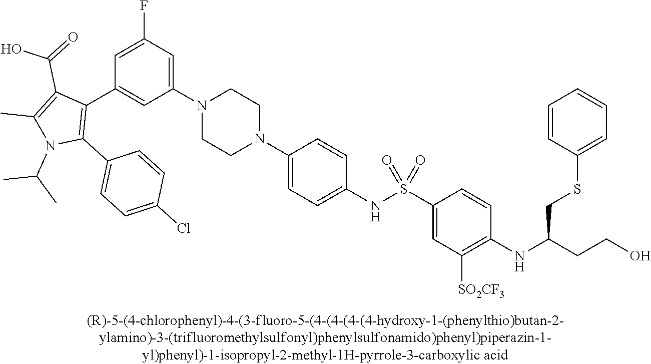 |
| 33 | 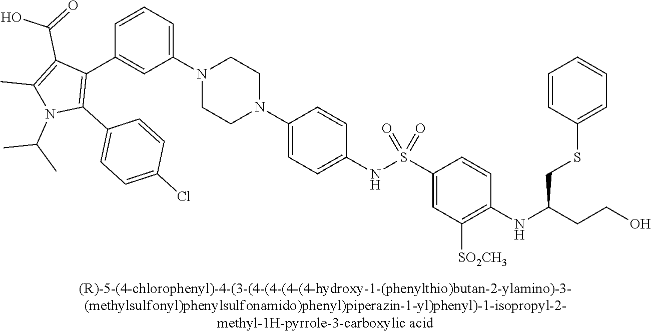 [SMILES: Cc1c(C(=O)O)c(-c2cccc(N3CCN(c4ccc(NS(=O)(=O)c5ccc(N[C@H](CCO)CSc6ccccc6)c(S(C)(=O)=O)c5)cc4)CC3)c2)c(-c2ccc(Cl)cc2)n1C(C)C] |
| 34 | 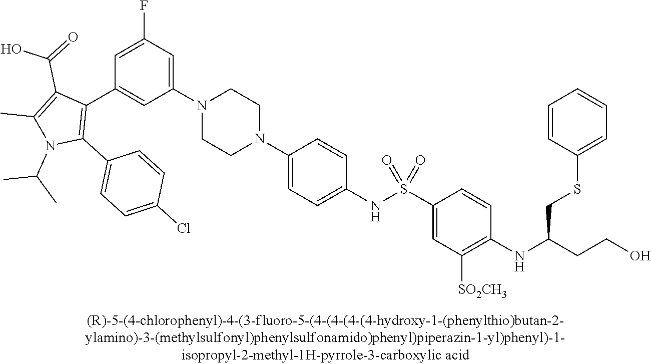 [SMILES: Cc1c(C(=O)O)c(-c2cc(F)cc(N3CCN(c4ccc(NS(=O)(=O)c5ccc(N[C@H](CCO)CSc6ccccc6)c(S(C)(=O)=O)c5)cc4)CC3)c2)c(-c2ccc(Cl)cc2)n1C(C)C] |
| 35 | 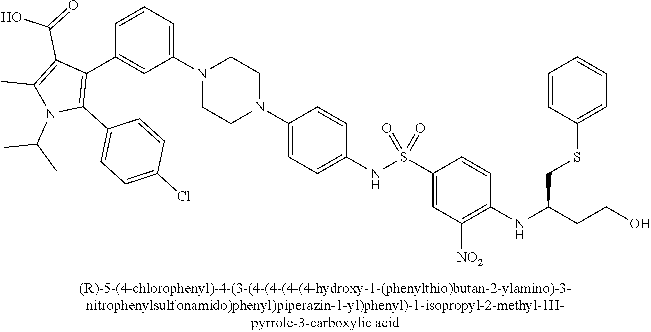 [SMILES: Cc1c(C(=O)O)c(-c2cccc(N3CCN(c4ccc(NS(=O)(=O)c5ccc(N[C@H](CCO)CSc6ccccc6)c([N+](=O)[O-])c5)cc4)CC3)c2)c(-c2ccc(Cl)cc2)n1C(C)C] |
| 36 | 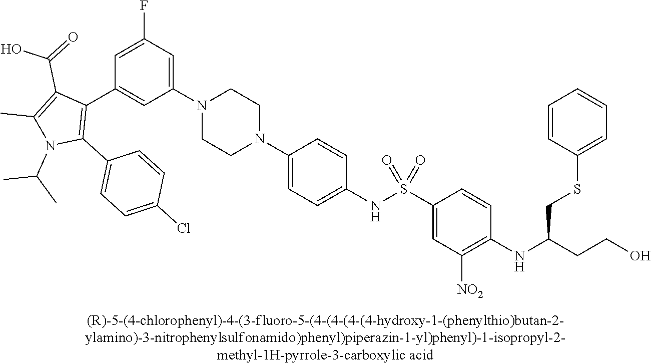 [SMILES: Cc1c(C(=O)O)c(-c2cc(F)cc(N3CCN(c4ccc(NS(=O)(=O)c5ccc(N[C@H](CCO)CSc6ccccc6)c([N+](=O)[O-])c5)cc4)CC3)c2)c(-c2ccc(Cl)cc2)n1C(C)C] |

Another class of exemplary Bcl inhibitors includes acyl benzylamines that have the following structure:

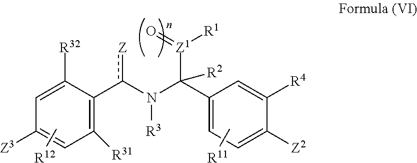

wherein:

Z¹ is C or S;

n is 1 or 2 wherein when Z¹ is C, n is 1;

R¹ is selected from R²¹, OH, OR²¹, NH₂ and NR²¹R²²;

R² is selected from H and R²¹;

or R¹ and R² together with the atoms through which they are connected form a 5- or 6-membered carbocyclic or heterocyclic ring, optionally substituted with one or more R²³;

Z is O and R³² is R¹²; or Z and R³² together with the atoms through which they are connected form a 5- or 6-membered aryl or heteroaryl ring optionally substituted with one or more R¹² groups;

R³ is selected from hydrogen, alkyl and substituted alkyl;

R⁴ is selected from hydrogen, alkyl, substituted alkyl, nitro, alkylsulfonyl, substituted alkylsulfonyl, alkylsulfinyl, substituted alkylsulfinyl, cyano, alkylcarbonyl, substituted alkylcarbonyl, C(O)OH, C(O)NH₂, halogen, SO₂NH₂, alkylaminosulfonyl, substituted alkylaminosulfonyl, alkylsulfonylamino and substituted alkylsulfonylamino, alkoxycarbonyl and substituted alkoxycarbonyl;

each R²¹ is independently selected from alkyl and substituted alkyl;

R²² is selected from hydrogen, alkyl and substituted alkyl;

each R²³ is independently selected from alkyl, substituted alkyl, hydroxyl, halogen, alkoxy and substituted alkoxy;

Z² is selected from —NR⁵R⁶, hydrogen, alkyl, substituted alkyl, alkenyl, substituted alkenyl, alkoxy, substituted alkoxy, alkylsulfanyl, substituted alkylsulfanyl, alkynyl, substituted alkynyl, aryl, substituted aryl, arylalkoxy, substituted arylalkoxy, aryloxy, substituted aryloxy, aryloxyalkoxy, substituted aryloxyalkoxy, arylsulfanyl, substituted arylsulfanyl, arylsulfanylalkoxy, substituted arylsulfanylalkoxy, cycloalkylalkoxy, substituted cycloalkylalkoxy, cycloalkyloxy, substituted cycloalkyloxy, halogen, carbonyloxy, haloalkoxy, haloalkyl, hydroxy and nitro;

R⁵ and R⁶ are independently selected from hydrogen, alkyl and substituted alkyl;

Z³ is selected from —NR⁵R⁶, aryl, substituted aryl, heteroaryl, substituted heteroaryl, carbocycle, substituted carbocycle, heterocycle and substituted heterocycle;

R¹¹ and R¹² are each one or more optional substituents each independently selected from alkyl, substituted alkyl, alkoxy, substituted alkoxy, halogen, cyano, nitro, carboxy, C(O)NH₂, SO₂NH₂, sulfonate, hydroxyl, alkylsulfonyl, substituted alkylsulfonyl, alkylaminosulfonyl, substituted alkylaminosulfonyl, alkylsulfonylamino, substituted alkylsulfonylamino, alkoxycarbonyl, substituted alkoxycarbonyl and —NR⁵R⁶; and

R³¹ is selected from H, R¹² and L³-Y³ wherein L³ is a linker and Y³ is selected from aryl, substituted aryl, heteroaryl and substituted heteroaryl.

Another class of exemplary Bcl inhibitors includes phosphonamidates that have the following structure:

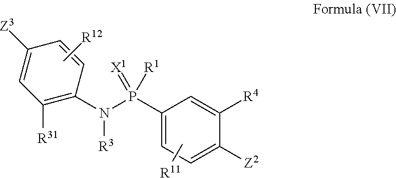

wherein:

X¹ is O or S;

R¹ is selected from SR²¹, OR²¹, and NR²¹R²²;

R³ is selected from hydrogen, alkyl and substituted alkyl;

R⁴ is selected from hydrogen, alkyl, substituted alkyl, nitro, alkylsulfonyl, substituted alkylsulfonyl, alkylsulfinyl, substituted alkylsulfinyl, cyano, alkylcarbonyl, substituted alkylcarbonyl, C(O)OH, C(O)NH₂, halogen, SO₂NH₂, alkylaminosulfonyl, substituted alkylaminosulfonyl, alkylsulfonylamino and substituted alkylsulfonylamino, alkanoyl, substituted alkanoyl, alkylaminocarbonyl, substituted alkylaminocarbonyl, alkyloxycarbonyl and substituted alkyloxycarbonyl;

R²¹ and R²² are independently selected from hydrogen, alkyl and substituted alkyl;

or R²¹ and R²² together with the N atom through which they are connected form a 5- or 6-membered heterocyclic ring, optionally substituted with one or more R²³;

Z² is selected from —NR⁵R⁶, hydrogen, alkyl, substituted alkyl, alkenyl, substituted alkenyl, alkoxy, substituted alkoxy, alkylsulfanyl, substituted alkylsulfanyl, alkynyl, substituted alkynyl, aryl, substituted aryl, heteroaryl, substituted heteroaryl, carbocycle, substituted carbocycle, heterocycle, substituted heterocycle, arylalkoxy, substituted arylalkoxy, aryloxy, substituted aryloxy, aryloxyalkoxy, substituted aryloxyalkoxy, arylsulfanyl, substituted arylsulfanyl, arylsulfanylalkoxy, substituted arylsulfanylalkoxy, cycloalkylalkoxy, substituted cycloalkylalkoxy, cycloalkyloxy, substituted cycloalkyloxy, halogen, carbonyloxy, haloalkoxy, haloalkyl, hydroxy and nitro;

Z³ is selected from heterocycle, substituted heterocycle, —NR⁵R⁶, aryl, substituted aryl, heteroaryl, substituted heteroaryl, carbocycle and substituted carbocycle;

R⁵ and R⁶ are independently selected from hydrogen, alkyl and substituted alkyl;

R¹¹ and R¹² are each one or more optional substituents each independently selected from alkyl, substituted alkyl, alkoxy, substituted alkoxy, halogen, cyano, nitro, carboxy, C(O)NH₂, SO₂NH₂, sulfonate, hydroxyl, alkylsulfonyl, substituted alkylsulfonyl, alkylaminosulfonyl, substituted alkylaminosulfonyl, alkylsulfonylamino, substituted alkylsulfonylamino, alkyloxycarbonyl, substituted alkyloxycarbonyl and —NR⁵R⁶; and

R³¹ is selected from H, R¹² and L³-Y³ wherein L³ is a linker and Y³ is selected from aryl, substituted aryl, heteroaryl and substituted heteroaryl.

Another class of exemplary Bcl inhibitors includes phospholidines that have the following structure:

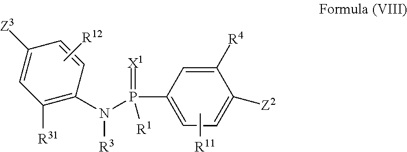

wherein:

X¹ is O or S;

R¹ and R³ together with the N and P atoms through which they are connected form a 5-, 6- or 7-membered heterocyclic ring, optionally substituted with one or more R²³;

R⁴ is selected from hydrogen, alkyl, substituted alkyl, nitro, alkylsulfonyl (e.g., CH₃SO₂—), substituted alkylsulfonyl (e.g., CF₃SO₂—), alkylsulfinyl, substituted alkylsulfinyl, cyano, C(O)OH, C(O)NH₂, halogen, SO₂NH₂, alkylaminosulfonyl, substituted alkylaminosulfonyl, alkylsulfonylamino and substituted alkylsulfonylamino, alkanoyl, substituted alkanoyl, alkylaminocarbonyl, substituted alkylaminocarbonyl, alkyloxycarbonyl and substituted alkyloxycarbonyl;

R²² is selected from hydrogen, alkyl and substituted alkyl;

each R²³ is independently selected from alkyl, substituted alkyl, —CONH₂, COOH, CONHR²², hydroxyl, halogen, alkoxy and substituted alkoxy;

Z² is selected from —NR⁵R⁶, hydrogen, alkyl, substituted alkyl, alkenyl, substituted alkenyl, alkoxy, substituted alkoxy, alkylsulfanyl, substituted alkylsulfanyl, alkynyl, substituted alkynyl, aryl, substituted aryl, heteroaryl, substituted heteroaryl, carbocycle, substituted carbocycle, heterocycle, substituted heterocycle, arylalkoxy, substituted arylalkoxy, aryloxy, substituted aryloxy, aryloxyalkoxy, substituted aryloxyalkoxy, arylsulfanyl, substituted arylsulfanyl, arylsulfanylalkoxy, substituted arylsulfanylalkoxy, cycloalkylalkoxy, substituted cycloalkylalkoxy, cycloalkyloxy, substituted cycloalkyloxy, halogen, carbonyloxy, haloalkoxy, haloalkyl, hydroxy and nitro;

Z³ is selected from heterocycle, substituted heterocycle, —NR⁵R⁶, aryl, substituted aryl, heteroaryl, substituted heteroaryl, carbocycle and substituted carbocycle;

R⁵ and R⁶ are independently selected from hydrogen, alkyl and substituted alkyl;

R¹¹ and R¹² are each one or more optional substituents each independently selected from alkyl, substituted alkyl, alkoxy, substituted alkoxy, halogen, cyano, nitro, carboxy, C(O)NH₂, SO₂NH₂, sulfonate, hydroxyl, alkylsulfonyl, substituted alkylsulfon24yl, alkylaminosulfonyl, substituted alkylaminosulfonyl, alkylsulfonylamino, substituted alkylsulfonylamino, alkyloxycarbonyl, substituted alkyloxycarbonyl and —NR⁵R⁶; and

R³¹ is selected from H, R¹² and L³-Y³ wherein L³ is a linker and Y³ is selected from aryl, substituted aryl, heteroaryl and substituted heteroaryl.

Another class of exemplary Bcl inhibitors includes acyl phosphoamidates that have the following structure:

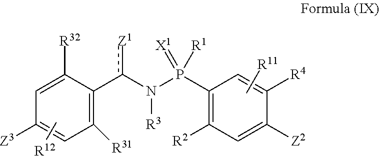

wherein:

X¹ is O or S;

R¹ is selected from SH, SR²¹, OH, OR²¹, NH₂ and NR²¹R²²;

R² is selected from H and R¹¹; or R¹ and R² together with the atoms through which they are connected form a 5- or 6-membered heterocyclic ring optionally substituted with one or more R²³;

Z¹ is O and R³² is R¹²; or Z¹ and R³² together with the atoms through which they are connected form a 5- or 6-membered fused carbocyclic, heterocyclic, aryl or heteroaryl ring optionally substituted with one or more R¹² groups;

R³ is selected from hydrogen, alkyl and substituted alkyl;

R⁴ is selected from hydrogen, alkyl, substituted alkyl, nitro, alkylsulfonyl (e.g., CH₃SO₂—), substituted alkylsulfonyl (e.g., CF₃SO₂—), alkylsulfinyl, substituted alkylsulfinyl, cyano, alkylcarbonyl, substituted alkylcarbonyl, C(O)OH, C(O)NH₂, halogen, SO₂NH₂, alkylaminosulfonyl, substituted alkylaminosulfonyl, alkylsulfonylamino and substituted alkylsulfonylamino, alkyloxycarbonyl and substituted alkyloxycarbonyl;

each R²¹ is independently selected from alkyl and substituted alkyl;

R²² is selected from hydrogen, alkyl and substituted alkyl; or R²¹ and R²² together with the N atom through which they are connected form a 5- or 6-membered heterocyclic ring, optionally substituted with one or more R²³;

each R²³ is independently selected from alkyl, substituted alkyl, hydroxyl, halogen, alkoxy and substituted alkoxy;

Z² is selected from —NR⁵R⁶, hydrogen, alkyl, substituted alkyl, alkenyl, substituted alkenyl, alkoxy, substituted alkoxy, alkylsulfanyl, substituted alkylsulfanyl, alkynyl, substituted alkynyl, aryl, substituted aryl, arylalkoxy, substituted arylalkoxy, aryloxy, substituted aryloxy, aryloxyalkoxy, substituted aryloxyalkoxy, arylsulfanyl, substituted arylsulfanyl, arylsulfanylalkoxy, substituted arylsulfanylalkoxy, cycloalkylalkoxy, substituted cycloalkylalkoxy, cycloalkyloxy, substituted cycloalkyloxy, halogen, carbonyloxy, haloalkoxy, haloalkyl, hydroxy and nitro;

R⁵ and R⁶ are independently selected from hydrogen, alkyl and substituted alkyl;

Z³ is selected from —NR⁵R⁶, aryl, substituted aryl, heteroaryl, substituted heteroaryl, carbocycle, substituted carbocycle, heterocycle and substituted heterocycle;

R¹¹ and R¹² are each independently one or more optional substituents each independently selected from alkyl, substituted alkyl, alkoxy, substituted alkoxy, halogen, cyano, nitro, carboxy, C(O)NH₂, SO₂NH₂, sulfonate, hydroxyl, alkylsulfonyl, substituted alkylsulfonyl, alkylaminosulfonyl, substituted alkylaminosulfonyl, alkylsulfonylamino, substituted alkylsulfonylamino, alkyloxycarbonyl, substituted alkyloxycarbonyl and —NR⁵R⁶; and

R³¹ is selected from H, R¹² and L³-Y³ wherein L³ is a linker and Y³ is selected from aryl, substituted aryl, heteroaryl and substituted heteroaryl.

In some embodiments, the Bcl inhibitor is BH3I-1, which is described in PCT Publication WO 2018/033128 and has the following chemical structure:

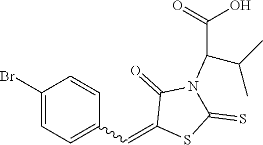 [SMILES: CC(C)C(C(=O)O)N1C(=O)C(=Cc2ccc(Br)cc2)SC1=S]

In some embodiments, the Bcl inhibitor is Sabutoclax (BI-97C1), which is described in PCT Publication WO 2018/033128 and has the following structure:

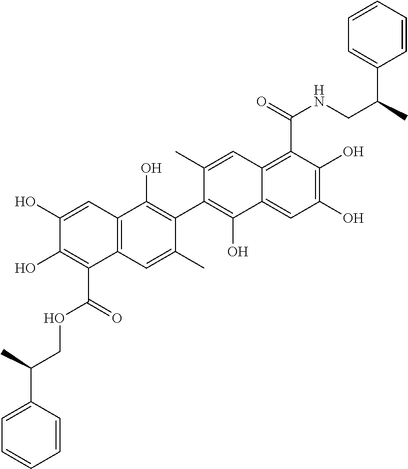 [SMILES: Cc1cc2c(C(=O)NC[C@H](C)c3ccccc3)c(O)c(O)cc2c(O)c1-c1c(C)cc2c(C(=O)OC[C@H](C)c3ccccc3)c(O)c(O)cc2c1O]

In some embodiments, the Bcl inhibitor is A-107250, which has the following structure:

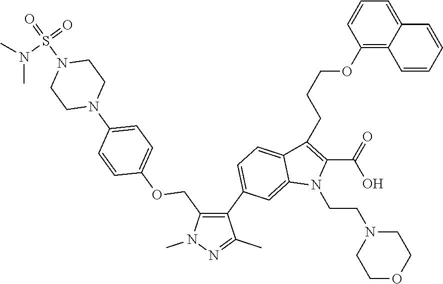 [SMILES: Cc1nn(C)c(COc2ccc(N3CCN(S(=O)(=O)N(C)C)CC3)cc2)c1-c1ccc2c(CCCOc3cccc4ccccc34)c(C(=O)O)n(CCN3CCOCC3)c2c1]

In some embodiments, the Bcl inhibitor is S63845, which is described in Cancer Cell, 2016, 30(6), 834-835, doi:10.1016/j.ccell.2016.11.016 and has the following structure:

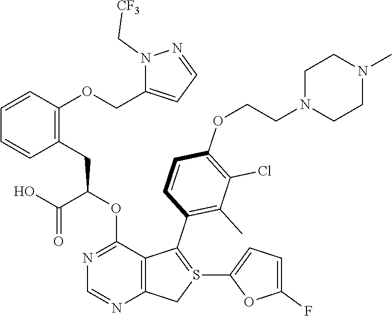

In some embodiments, the Bcl inhibitor is MAIM1, which is described in Nat. Struct. Mol. Biol., 2016, 23(6), 600-607, doi:10.1038/nsmb.3223 and has the chemical structure:

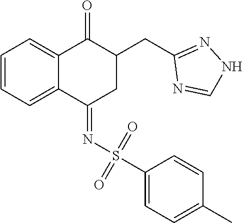 [SMILES: Cc1ccc(S(=O)(=O)/N=C2\CC(Cc3nc[nH]n3)C(=O)c3ccccc32)cc1]

In some embodiments, the Bcl inhibitor is UM-36, which is described in FEBS Lett., 2016, 591(1), 240-251, doi:10.1002/1873-3468.12497 and has the chemical structure:

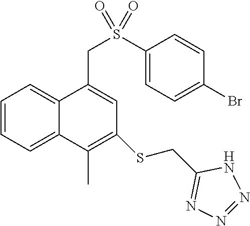 [SMILES: Cc1c(SCc2nnn[nH]2)cc(CS(=O)(=O)c2ccc(Br)cc2)c2ccccc12]

In some embodiments, the Bcl inhibitor is EU5346 (ML311), which is described in FEBS Lett., 2016, 591(1), 240-251, doi:10.1002/1873-3468.12497 and has the chemical structure:

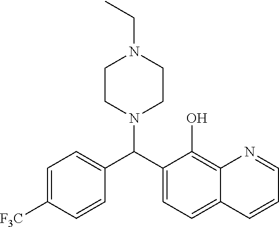 [SMILES: CCN1CCN(C(c2ccc(C(F)(F)F)cc2)c2ccc3cccnc3c2O)CC1]

In other embodiments, the Bcl inhibitor is APG-1252 (BM-1252). Additional disclosure related to APG-1252 (BM-1252) can be found in European Journal of Cancer, 50, 6, 109-110, doi:10.1016/S0959-8049(14)70464-2.

In some embodiments, the Bcl inhibitor is TM-1205, which is described in J. Med. Chem., 2008, 51, 717-720, doi:10.1021/jm701358v and has the following structure:

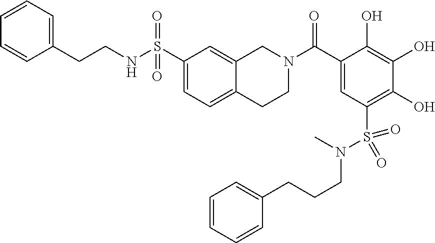 [SMILES: CN(CCCc1ccccc1)S(=O)(=O)c1cc(C(=O)N2CCc3ccc(S(=O)(=O)NCCc4ccccc4)cc3C2)c(O)c(O)c1O]

In some embodiments, the Bcl inhibitor is a pyrogallol. In some cases, the pyrogallol is an acylpyrogallol. In some embodiments, the Bcl inhibitor is TM-179. Additional disclosure related to pyrogallols, acylpyrogallols, and TM-179 can be found in J. Med. Chem., 2007, 50, 8, 1723-1726, doi:10.1021/jm0614001. TM-179 has the following structure:

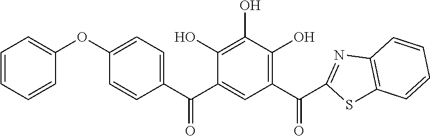 [SMILES: O=C(c1ccc(Oc2ccccc2)cc1)c1cc(C(=O)c2nc3ccccc3s2)c(O)c(O)c1O]

In some embodiments, the Bcl inhibitor is BM-903, which is described in J. Med. Chem., 2013, 56, 3048-3067, doi:10.1021/jm4001105 and has the structure:

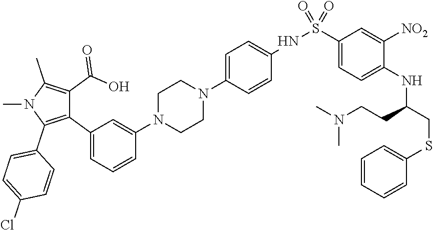 [SMILES: Cc1c(C(=O)O)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C]

In some embodiments, the Bcl inhibitor is BM-956, which is described in J. Med. Chem., 2013, 56, 3048-3067, doi:10.1021/jm4001105 and has the structure:

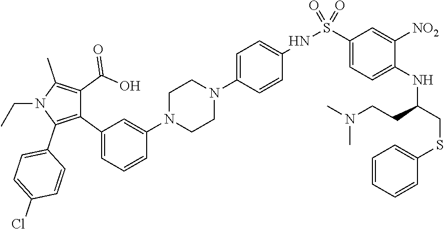 [SMILES: CCn1c(C)c(C(=O)O)c(-c2cccc(N3CCN(c4ccc(NS(=O)(=O)c5ccc(N[C@H](CCN(C)C)CSc6ccccc6)c([N+](=O)[O-])c5)cc4)CC3)c2)c1-c1ccc(Cl)cc1]

In some embodiments, the Bcl inhibitor is BM-1074, which is described in J. Med. Chem., 2013, 56, 3048-3067, doi:10.1021/jm4001105 and has the structure:

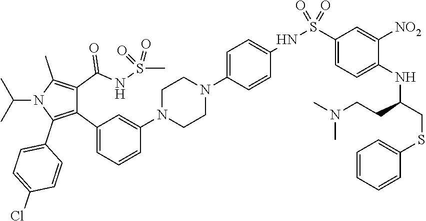 [SMILES: Cc1c(C(=O)NS(C)(=O)=O)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C(C)C]

In some embodiments, the Bcl inhibitor is (−)BI97D6, which is described in Blood, 2015, 126, 363-372, doi:10.1182/blood-2014-10-604975 and has the structure:

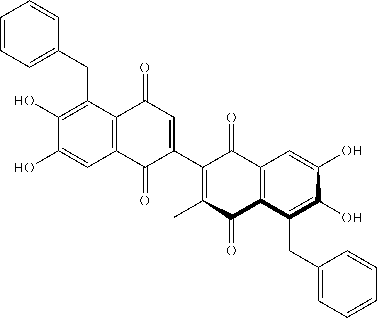

In some embodiments, the Bcl inhibitor is AB141523 (2-methoxy-antimycin A3), which is described in PCT Publication WO 2018/033128 and has the structure:

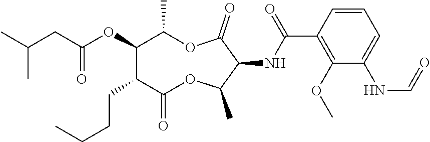 [SMILES: CCCC[C@H]1C(=O)O[C@H](C)[C@H](NC(=O)c2cccc(NC=O)c2OC)C(=O)O[C@@H](C)[C@@H]1OC(=O)CC(C)C]

In some embodiments, the Bcl inhibitor is JY-1-106, which is described in WO 2018/052120 and has the structure:

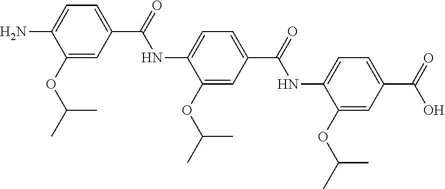 [SMILES: CC(C)Oc1cc(C(=O)Nc2ccc(C(=O)Nc3ccc(C(=O)O)cc3OC(C)C)cc2OC(C)C)ccc1N]

In some embodiments, the Bcl inhibitor is a gossypol. The gossypol can be, for example, R-(−)-gossypol (AT-101), S-(−)-gossypol, (−)-gossypol ((−)-BL 193), (+)-gossypol ((+)-BL 193), apogossypol, or gossypolone. In some cases, the Bcl inhibitor composition comprises a gossypol and acetic acid, or a gossypol and a pharmaceutically acceptable salt of acetic acid. Additional disclosure related to gossypol (BL 193) and R-(−)-gossypol (AT-101) can be found at Expert Opin. Ther. Targets, 2013, 17(1), 61-75, doi:10.1517/14728222.2013.733001. Additional disclosure related to apogossypol can be found in PCT Publication WO 2018/033128. Additional disclosure related to gossypolone can be found in PCT Publication WO 2006/050447.

The structure of R-(−)-gossypol (AT-101) is:

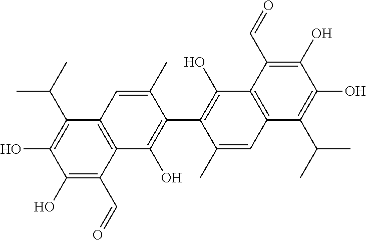 [SMILES: Cc1cc2c(C(C)C)c(O)c(O)c(C=O)c2c(O)c1-c1c(C)cc2c(C(C)C)c(O)c(O)c(C=O)c2c1O]

The structure of apogossypol is:

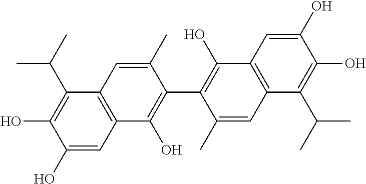 [SMILES: Cc1cc2c(C(C)C)c(O)c(O)cc2c(O)c1-c1c(C)cc2c(C(C)C)c(O)c(O)cc2c1O]

The structure of gossypolone is:

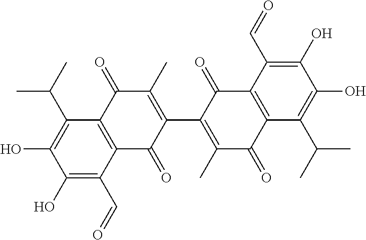 [SMILES: CC1=C(C2=C(C)C(=O)c3c(c(C=O)c(O)c(O)c3C(C)C)C2=O)C(=O)c2c(C=O)c(O)c(O)c(C(C)C)c2C1=O]

In some embodiments, the Bcl inhibitor is A-1155463, which has the structure:

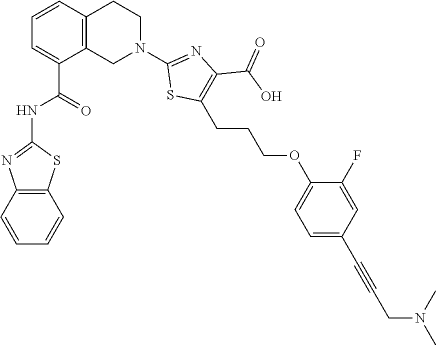 [SMILES: CN(C)CC#Cc1ccc(OCCCc2sc(N3CCc4cccc(C(=O)Nc5nc6ccccc6s5)c4C3)nc2C(=O)O)c(F)c1]

In some embodiments, the Bcl inhibitor is A-1331852, which is described in PCT Publication WO 2018/033128 and has the structure:

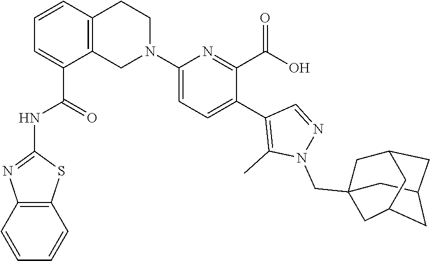 [SMILES: Cc1c(-c2ccc(N3CCc4cccc(C(=O)Nc5nc6ccccc6s5)c4C3)nc2C(=O)O)cnn1CC12CC3CC(CC(C3)C1)C2]

In some embodiments, the Bcl inhibitor is WEHI-539, which is described in PCT Publication WO 2018/033128 and has the structure:

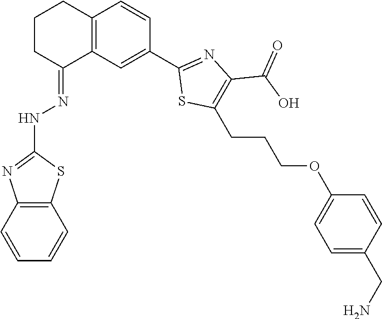 [SMILES: NCc1ccc(OCCCc2sc(-c3ccc4c(c3)/C(=N/Nc3nc5ccccc5s3)CCC4)nc2C(=O)O)cc1]

In some embodiments, the Bcl inhibitor is BXI-61 (NSC354961, 3-[(9-Amino-7-ethoxyacridin-3-yl)diazenyl]pyridine-2,6-diamine) or BXI-72 (NSC334072, bisbenzimide). Additional disclosure related to BXI-61 and BXI-72 can found at Cancer Res., 2013, 73(17), 5485-96, doi:10.1158/0008-5472.CAN-12-2272. BXI-61 has the structure:

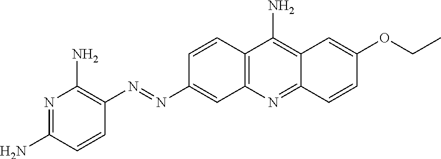 [SMILES: CCOc1ccc2nc3cc(/N=N/c4ccc(N)nc4N)ccc3c(N)c2c1]

BXI-72 has the structure:

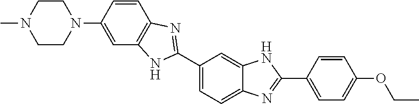 [SMILES: CCOc1ccc(-c2nc3ccc(-c4nc5ccc(N6CCN(C)CC6)cc5[nH]4)cc3[nH]2)cc1]

### Bcl-xL Inhibitors

This section provides compounds that constitute a means for inhibiting Bcl-xL. They are suitable for testing as senolytic agents in combination with Mcl-1 family inhibitors, according to this invention.

One class of exemplary Bcl-xL inhibitors includes, for example, A-1155463, A-1331852, A-385358, AB141523 (2-methoxy-antimycin A3), BH3I-1, gossypols, gossypol (BL 193), (−)-gossypol ((−) BL 193), (+)-gossypol ((+)BL 193), R-(−)-gossypol (AT-101), S-(−)-gossypol, apogossypol, gossypolone, Sabutoclax (BI-97C1), WEHI-539, and pharmaceutically acceptable salts thereof.

In some embodiments, the Bcl-xL inhibitor is A-1155463, which has the structure:

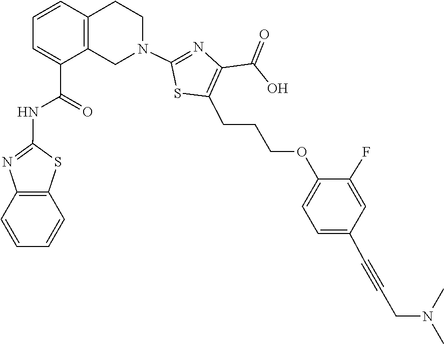 [SMILES: CN(C)CC#Cc1ccc(OCCCc2sc(N3CCc4cccc(C(=O)Nc5nc6ccccc6s5)c4C3)nc2C(=O)O)c(F)c1]

In some embodiments, the Bcl-xL inhibitor is A-1331852, which is described in PCT Publication WO 2018/033128 and has the structure:

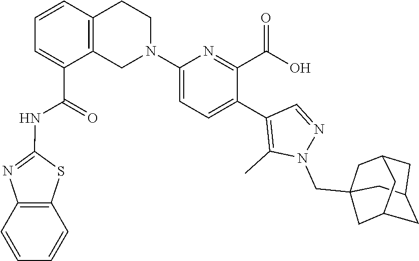 [SMILES: Cc1c(-c2ccc(N3CCc4cccc(C(=O)Nc5nc6ccccc6s5)c4C3)nc2C(=O)O)cnn1CC12CC3CC(CC(C3)C1)C2]

In some embodiments, the Bcl-xL inhibitor is AB141523 (2-methoxy-antimycin A3), which is described in PCT Publication WO 2018/033128 and has the structure:

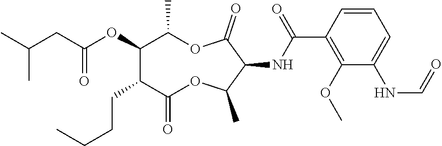 [SMILES: CCCC[C@H]1C(=O)O[C@H](C)[C@H](NC(=O)c2cccc(NC=O)c2OC)C(=O)O[C@@H](C)[C@@H]1OC(=O)CC(C)C]

In some embodiments, the Bcl-xL inhibitor is BH3I-1, which is described in PCT Publication WO 2018/033128 and has the following chemical structure:

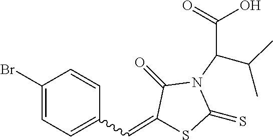 [SMILES: CC(C)C(C(=O)O)N1C(=O)C(=Cc2ccc(Br)cc2)SC1=S]

In some embodiments, the Bcl-xL inhibitor is a gossypol. The gossypol can be, for example, gossypol (BL 193), (−)-gossypol ((−)BL 193), (+)-gossypol ((+)BL 193), R-(−)-gossypol (AT-101), S-(−)-gossypol, apogossypol, or gossypolone. In some cases, the Bcl-xL inhibitor composition comprises a gossypol and acetic acid, or a gossypol and a pharmaceutically acceptable salt of acetic acid. Additional disclosure related to gossypol (BL 193) and R-(−)-gossypol (AT-101) can be found at Expert Opin. Ther. Targets, 2013, 17(1), 61-75, doi:10.1517/14728222.2013.733001. Additional disclosure related to apogossypol can be found in PCT Publication WO 2018/033128. Additional disclosure related to gossypolone can be found in PCT Publication WO 2006/050447.

The structure of R-(−)-gossypol (AT-101) is:

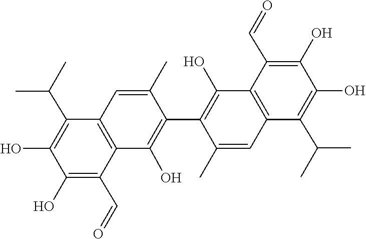 [SMILES: Cc1cc2c(C(C)C)c(O)c(O)c(C=O)c2c(O)c1-c1c(C)cc2c(C(C)C)c(O)c(O)c(C=O)c2c1O]

The structure of apogossypol is:

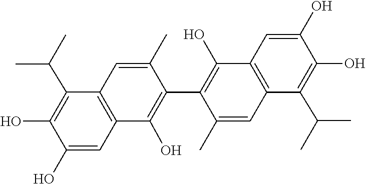 [SMILES: Cc1cc2c(C(C)C)c(O)c(O)cc2c(O)c1-c1c(C)cc2c(C(C)C)c(O)c(O)cc2c1O]

The structure of gossypolone is:

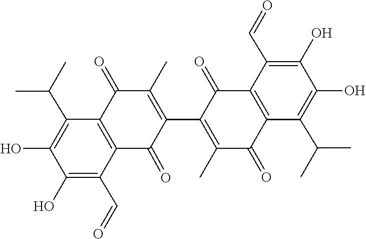 [SMILES: CC1=C(C2=C(C)C(=O)c3c(c(C=O)c(O)c(O)c3C(C)C)C2=O)C(=O)c2c(C=O)c(O)c(O)c(C(C)C)c2C1=O]

In some embodiments, the Bcl-xL inhibitor is Sabutoclax (BI-97C1), which is described in PCT Publication WO 2018/033128 and has the following structure:

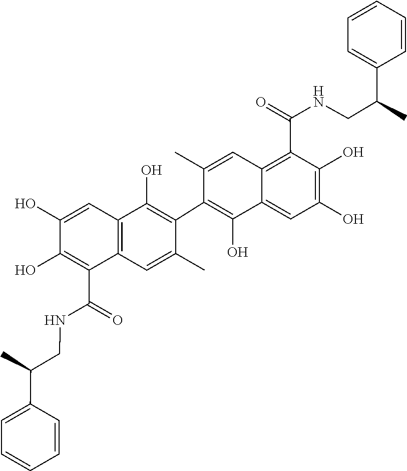 [SMILES: Cc1cc2c(C(=O)NC[C@H](C)c3ccccc3)c(O)c(O)cc2c(O)c1-c1c(C)cc2c(C(=O)NC[C@H](C)c3ccccc3)c(O)c(O)cc2c1O]

In some embodiments, the Bcl-xL inhibitor is WEHI-539, which is described in PCT Publication WO 2018/033128 and has the structure:

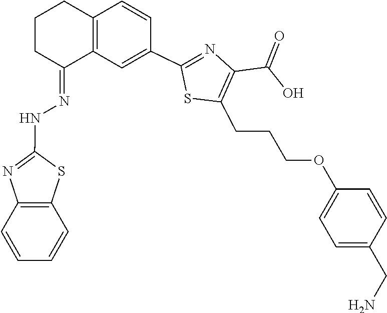 [SMILES: NCc1ccc(OCCCc2sc(-c3ccc4c(c3)/C(=N/Nc3nc5ccccc5s3)CCC4)nc2C(=O)O)cc1]

### Mcl-1 Inhibitors

This section provides compounds that constitute a means for inhibiting Mcl-1. They are suitable for testing as senolytic agents in combination with Bcl family inhibitors, according to this invention.

It has now been discovered that the combination of the Bcl inhibitors and the Mcl-1 inhibitors put forth in this disclosure provide an enhanced senolytic potency to promote the selective apoptosis of senescent cells.

One class of exemplary Mcl-1 inhibitors includes, for example, 483-LM, 347-PXN-0209, A-107250, A-1210477, AMG-176, AZ-Mcl1, AZD-5991, (−)BI97D6, gambogic acid, EU-517, EU5346 (ML311), EU-5148, gossypols, gossypol (BL 193), (−)-gossypol ((−)BL 193), (+)-gossypol ((+)BL 193), R-(−)-gossypol (AT-101), S-(−)-gossypol, apogossypol, gossypolone, JKY-5-037, KS-18, MAIM1, Maritoclax (marinopyrrol A), MI-223, MIM1, ONC-301, Pyridoclax (MR-29072), pyrogallols, acylpyrogallols, Ra-50072, S63845, S-64315 (MIK655), Sabutoclax (BI-97C1), SU 9516, TM-179, TW-37, UM-36, UMI-77, VU661013, or a pharmaceutically acceptable salt thereof.

In some embodiments, the Mcl-1 inhibitor has the formula:

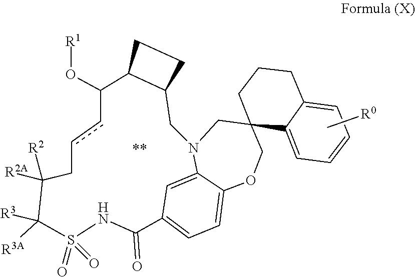

or a pharmaceutically acceptable salt thereof,
wherein:

the bond labeled as (**) is a single or double chemical bond which may be cis or trans;

R⁰ is a halo;

R¹ is H, C₁₋₆ alkyl, or —(CH₂CH₂O)ₙCH₃, wherein n is an integer from 1 to 4;

R² is H or C₁₋₆ alkyl;

R^{2A} is H or C₁₋₆ alkyl;

R³ is H or C₁₋₆ alkyl; and

R^{3A} is H, C₁₋₆ alkyl, C₃₋₆ cycloalkyl, or (CH₂)ₘ—C₃₋₆ cycloalkyl, wherein m is an integer from 1 to 4.

In some embodiments, the Mcl-1 inhibitor according to Formula (X) is AMG-176, which has the formula:

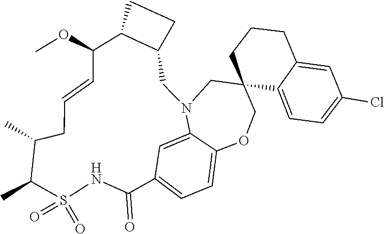 [SMILES: CO[C@@H]1/C=C/C[C@@H](C)[C@H](C)S(=O)(=O)NC(=O)c2ccc3c(c2)N(C[C@H]2CC[C@H]21)C[C@@]1(CCCc2cc(Cl)ccc21)CO3]

In some embodiments, the Mcl-1inhibitor is AZD-5991, which has the formula:

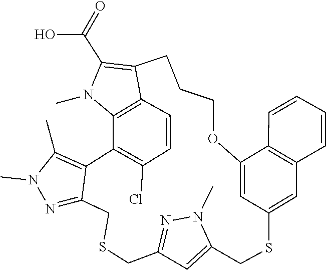 [SMILES: Cc1c2c(nn1C)CSCc1cc(n(C)n1)CSc1cc(c3ccccc3c1)OCCCc1c(C(=O)O)n(C)c3c-2c(Cl)ccc13]

In some embodiments, the Mcl-1inhibitor is S-64315 (MIK655), which has the formula:

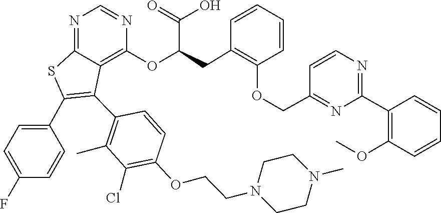 [SMILES: COc1ccccc1-c1nccc(COc2ccccc2C[C@@H](Oc2ncnc3sc(-c4ccc(F)cc4)c(-c4ccc(OCCN5CCN(C)CC5)c(Cl)c4C)c23)C(=O)O)n1]

In some embodiments, the Mcl-1inhibitor is Maritoclax (marinopyrrol A), which has the formula:

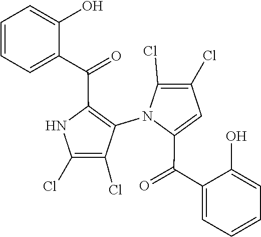 [SMILES: O=C(c1ccccc1O)c1[nH]c(Cl)c(Cl)c1-n1c(C(=O)c2ccccc2O)cc(Cl)c1Cl]

In some embodiments, the Mcl-1inhibitor is A-1210477, which is described in Cell Death Dis., 2015, 6, e1590, doi:10.1038/cddis.2014.561 and has the formula:

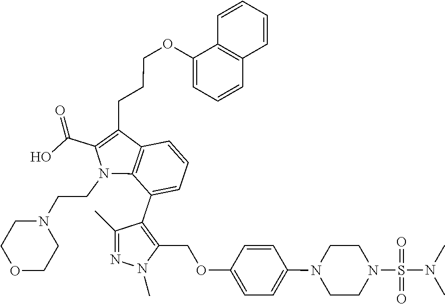 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCOCC3)c12]

In some embodiments, the Mcl-1inhibitor is gambogic acid, which has the formula:

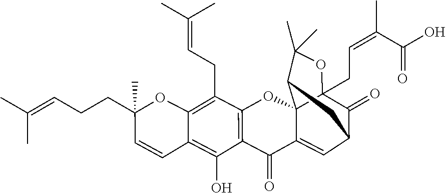 [SMILES: CC(C)=CCC[C@]1(C)C=Cc2c(O)c3c(c(CC=C(C)C)c2O1)O[C@]12C(=C[C@@H]4C[C@H]1C(C)(C)OC2(C/C=C(/C)C(=O)O)C4=O)C3=O]

In some embodiments, the Mcl-1inhibitor is UMI-77, which is described in Mol. Cancer Ther., 2014, 13(3), 565-575, doi:10.1158/1535-7163.MCT-12-0767 and has the formula:

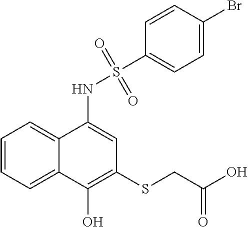 [SMILES: O=C(O)CSc1cc(NS(=O)(=O)c2ccc(Br)cc2)c2ccccc2c1O]

In some embodiments, the Mcl-1inhibitor is 483-LM, which is described in Cancer Research, 2017, Kump et al., doi:10.1158/1538-7445.AM2017-1173 and has the structure:

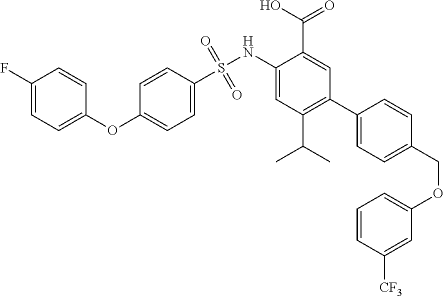 [SMILES: CC(C)c1cc(NS(=O)(=O)c2ccc(Oc3ccc(F)cc3)cc2)c(C(=O)O)cc1-c1ccc(COc2cccc(C(F)(F)F)c2)cc1]

In some embodiments, the Mcl-1inhibitor is EU5346 (ML311), which is described in FEBS Lett., 2017, 591(1), 240-251, doi:10.1002/1873-3468.12497 and has the structure:

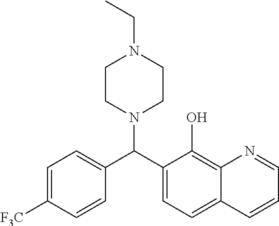 [SMILES: CCN1CCN(C(c2ccc(C(F)(F)F)cc2)c2ccc3cccnc3c2O)CC1]

In some embodiments, the Mcl-1inhibitor is TW-37, which is described in Cancer Res., 2006, 66(17), 8698-8706, Zeitlin et al. and has the structure:

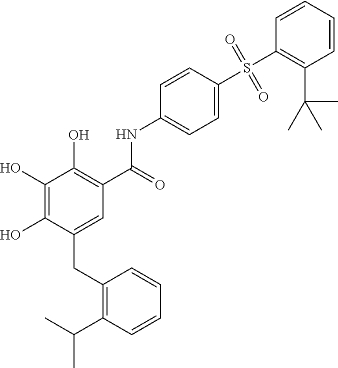 [SMILES: CC(C)c1ccccc1Cc1cc(C(=O)Nc2ccc(S(=O)(=O)c3ccccc3C(C)(C)C)cc2)c(O)c(O)c1O]

In some embodiments, the Mcl-1 inhibitor is Sabutoclax (BI-97C1), which is described in PCT Publication WO 2018/033128 and has the following structure:

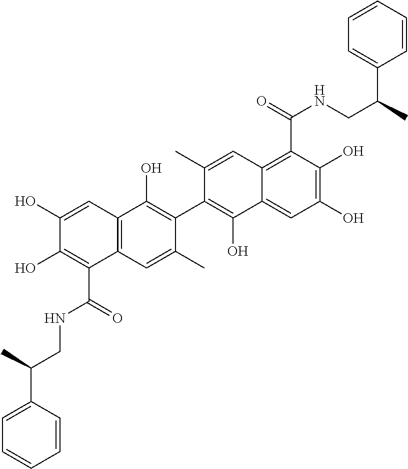 [SMILES: Cc1cc2c(C(=O)NC[C@H](C)c3ccccc3)c(O)c(O)cc2c(O)c1-c1c(C)cc2c(C(=O)NC[C@H](C)c3ccccc3)c(O)c(O)cc2c1O]

In some embodiments, the Mcl-1 inhibitor is a gossypol. The gossypol can be, for example, R-(−)-gossypol (AT-101), S-(−)-gossypol, (−)-gossypol ((−)-BL 193), (+)-gossypol ((+)-BL 193), apogossypol, or gossypolone. In some cases, the Mcl-1 inhibitor composition comprises a gossypol and acetic acid, or a gossypol and a pharmaceutically acceptable salt of acetic acid. Additional disclosure related to gossypol (BL 193) and R-(−)-gossypol (AT-101) can be found at Expert Opin. Ther. Targets, 2013, 17(1), 61-75, doi:10.1517/14728222.2013.733001. Additional disclosure related to apogossypol can be found in PCT Publication WO 2018/033128. Additional disclosure related to gossypolone can be found in PCT Publication WO 2006/050447.

The structure of R-(−)-gossypol (AT-101) is:

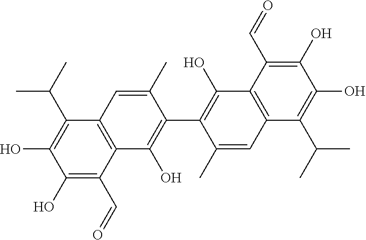 [SMILES: Cc1cc2c(C(C)C)c(O)c(O)c(C=O)c2c(O)c1-c1c(C)cc2c(C(C)C)c(O)c(O)c(C=O)c2c1O]

The structure of apogossypol is:

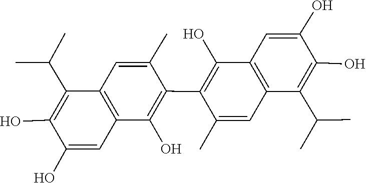 [SMILES: Cc1cc2c(C(C)C)c(O)c(O)cc2c(O)c1-c1c(C)cc2c(C(C)C)c(O)c(O)cc2c1O]

The structure of gossypolone is:

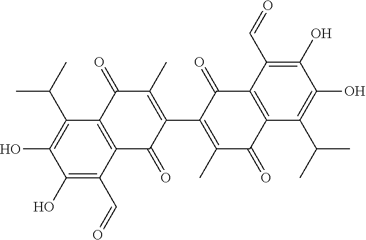 [SMILES: CC1=C(C2=C(C)C(=O)c3c(c(C=O)c(O)c(O)c3C(C)C)C2=O)C(=O)c2c(C=O)c(O)c(O)c(C(C)C)c2C1=O]

In some embodiments, the Mcl-1 inhibitor is AZ-Mcl1. Additional disclosure related to AZ-Mcl1 can be found in FEBS Lett., 2016, 591(1), 240-251, doi:10.1002/1873-3468.12497.

In some embodiments, the Mcl-1 inhibitor is (−)BI97D6, which is described in Blood, 2015, 126, 363-372, doi:10.1182/blood-2014-10-604975 and has the structure:

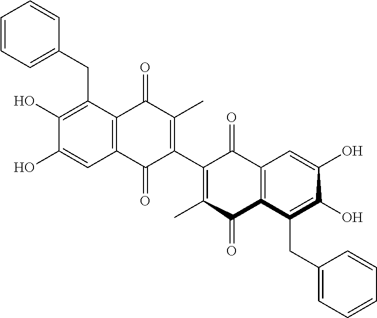

In some embodiments, the Mcl-1 inhibitor is a pyrogallol. In some cases, the pyrogallol is an acylpyrogallol. In some embodiments, the Mcl-1 inhibitor is TM-179. Additional disclosure related to pyrogallols, acylpyrogallols, and TM-179 can be found in J. Med. Chem., 2007, 50, 8, 1723-1726, doi:10.1021/jm0614001. TM-179 has the following structure:

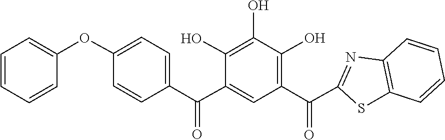 [SMILES: O=C(c1ccc(Oc2ccccc2)cc1)c1cc(C(=O)c2nc3ccccc3s2)c(O)c(O)c1O]

In some embodiments, the Mcl-1 inhibitor is VU661013, which is described in Cancer Discovery, 2018, Ramsey et al., doi:10.1158/2159-8290.CD-18-0140 and has the structure:

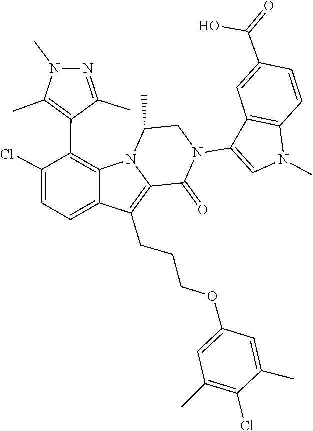 [SMILES: Cc1cc(OCCCc2c3n(c4c(-c5c(C)nn(C)c5C)c(Cl)ccc24)[C@H](C)CN(c2cn(C)c4ccc(C(=O)O)cc24)C3=O)cc(C)c1Cl]

In some embodiments, the Mcl-1 inhibitor is UM-36, which is described in FEBS Lett., 2016, 591(1), 240-251, doi:10.1002/1873-3468.12497 and has the chemical structure:

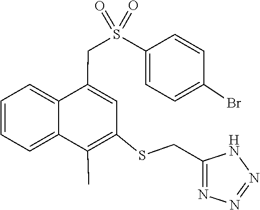 [SMILES: Cc1c(SCc2nnn[nH]2)cc(CS(=O)(=O)c2ccc(Br)cc2)c2ccccc12]

In some embodiments, the Mcl-1 inhibitor is MAIM1, which is described in Nat. Struct. Moi. Biol., 2016, 23(6), 600-607, doi:10.1038/nsmb.3223 and has the chemical structure:

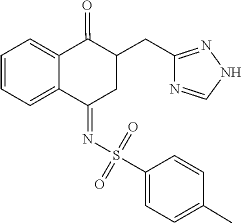 [SMILES: Cc1ccc(S(=O)(=O)/N=C2\CC(Cc3nc[nH]n3)C(=O)c3ccccc32)cc1]

In some embodiments, the Mcl-1 inhibitor is 563845, which is described in Cancer Cell, 2016, 30(6), 834-835, doi:10.1016/j.ccell.2016.11.016 and has the following structure:

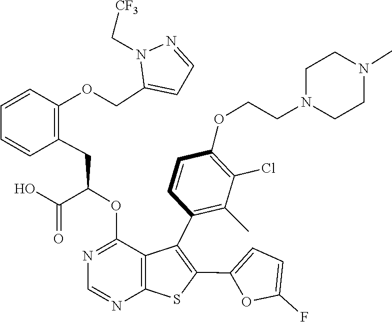

### Evaluating Compounds for Senolytic Activity

Bcl inhibitors and Mcl-1 inhibitors can be evaluated on the molecular level for their ability to perform in a way that indicates they are suitable senolytic combinations for use according to this invention.

For example, where the therapy includes triggering apoptosis of senescent cells by way of Bcl-2, Bcl-xL, Bcl-w, Mcl-1, or other Bcl family proteins, candidate Bcl inhibitor or Mcl-1 inhibitor compounds can be tested for their ability to inhibit binding between one or more Bcl and Mcl-1 proteins and their respective cognate ligand. As a non-limiting example, Example 2 provides an illustration of a homogeneous assay (an assay that does not require a separation step) based on oxygen channeling for purposes of determining by direct binding the ability of a candidate Bcl or Mcl-1 inhibitor to disrupt the binding of a cognate ligand to the Bcl family proteins of interest. As another non-limiting example, Example 6 provides a immunocapture and co-immunoprecipitation target engagement assay that can simultaneously detect both Bcl and Mcl-1 interactions with BIM in the presence of candidate Bcl and/or Mcl-1 inhibitors.

Candidate compounds can also be evaluated for an ability to kill senescent cells selectively. Cultured cells are contacted with the compound, and the degree of cytotoxicity or inhibition of the cells is determined. The ability of the compound to kill or inhibit senescent cells can be compared with the effect of the compound on normal cells that are freely dividing at low density, and normal cells that are in a quiescent state at high density. As non-limiting examples, Examples 3, 4 and 7, provide illustrations of selective senescent cell killing using various cell lines that are induced by irradiation to senesce: a primary human small airway epithelial cell (SAEC), or the primary human bronchial epithelial cells (HBEC), or the human fibroblast IMR90 cell line, or the human endothelial HUVEC cell line. Similar protocols are known and can be developed or optimized for testing the ability of candidate senolytic compounds to kill other senescent cell types.

Candidate senolytic combinations that are effective in selectively killing senescent cells in vitro can be further screened in animal models for particular diseases. As non-limiting examples, Examples 9-13 in the Experimental Section provide illustrations for pulmonary disease, osteoarthritis, glaucoma disease, diabetes-induced retinopathy, and atherosclerosis respectively.

### Determining Senolytic Synergy Between Bcl Inhibitor and Mcl-1 Inhibitor Combinations

Many of the effective combinations of Bcl and Mcl-1 inhibitors are attributable at least in part to functional synergy between the two compounds. According to current understanding (and without implying any limitation on the practice of the invention), the proteins Bcl-2, Bcl-xL, and Mcl-1 are all part of a mitochondrial pathway that regulate caspase 3, 6, and 7, leading to apoptosis. Synergy between Bcl inhibitors and Mcl-1 inhibitors may be direct or indirect, leading to enhanced inhibition, decreased regulation of caspase activity, and consequently an increase in apoptosis, leading to elimination of the senescent cell.

To quantify the degree of synergy of a combination of candidate senolytic agents, the combination response can be compared against an expected combination response, under the assumption of non-interaction calculated using a reference model (Tang J. et al. (2015) What is synergy? The saariselkä agreement revisited. Front. Pharmacol., 6, 181). Commonly-utilized reference models can include, for example, the highest single agent (HSA) model, where the synergy score quantifies the excess over the highest single drug response (Berenbaum M. C. (1989) What is synergy. Pharmacol. Rev., 41, 93-141); the Loewe additivity model, where the synergy score quantifies the excess over the expected response if the two drugs are the same compound (Loewe S. (1953) The problem of synergism and antagonism of combined drugs. ArzneimiettelForschung, 3, 286-290); the Bliss independence model, where the expected response is a multiplicative effect as if the two drugs act independently (Bliss C. I. (1939) The toxicity of poisons applied jointly. Ann. Appl. Biol., 26, 585-615); or the Zero interaction potency (ZIP) model, where the expected response corresponds to an additive effect as if the two drugs do not affect the potency of each other (Yadav B. et al. (2015) Searching for drug synergy in complex dose-response landscapes using an interaction potency model. Comput. Struct. Biotechnol. J., 13, 504-505).

To facilitate data processing of the senolytic dose-response matrices performed using different doses of tested combinations of Bcl inhibitors and Mcl-1 inhibitors on senescent cells, the user may employ an algorithm that uses key functions of R-package, called SynergyFinder. This algorithm is described by Ianevski A. et al. (2017) SynergyFinder: a web application for analyzing drug combination dose-response matrix data. Bioinformatics. August 1; 33(15): 2413-2415. The algorithm is publicly available from the Netherlands Translational Research Center, and can be accessed via the Internet. User instructions and tutorials of the SynergyFinder package have been published by He, Wennerberg, Aittokallio and Tang in 2016, updated 2018.

Unless explicitly stated or otherwise required, effective combinations of inhibitors according to this invention do not necessarily require measurable synergy at the target engagement level in experiments done in vitro in order to be effective for particular purposes in vivo. However, the user may find it useful to screen for effective combinations by calculating inhibition interactions according to the HAS model, the Loewe additivity model, the Bliss independence model, or the ZIP model. Reference in this disclosure to a delta (“δ”) synergy coefficient or index refers to the δ value calculated according to the ZIP model of Yadav et al., supra. Using this model, the larger the δ value, the stronger the synergistic senolysis. Any δ value larger than 0 shows positive synergy. The δ values given in the experimental sections below were calculated using the ZIP model.

Effective combinations of senolytic agents such as Bcl-xL or Bcl-2/xL inhibitors and Mcl-1 inhibitors according to this invention may have a δ value, or synergy coefficient that is greater than 5, 10, 15, 20, 30, 50, 80, or 150. Expressed in ranges, the synergy between such compounds may have δ values in the range of 1-500, 10-100, or 20-100.

### Formulation of Medicaments

Preparation and formulation of pharmaceutical agents for use according to this invention can incorporate standard technology, as described, for example, in the current edition of Remington: The Science and Practice of Pharmacy. The formulation will typically be optimized for administration to the target tissue, for example, by local administration, in a manner that enhances access of the active agent to the target senolytic cells and providing the optimal duration of effect, while minimizing side effects or exposure to tissues that are not involved in the condition being treated.

Pharmaceutical preparations for use in treating senescence-related conditions and other diseases can be prepared by mixing the candidate Bcl or Mcl-1 inhibitor with a pharmaceutically acceptable base or carrier and as needed one or more pharmaceutically acceptable excipients. Exemplary excipients and additives that can be used include surfactants (for example, polyoxyethylene and block copolymers); buffers and pH adjusting agents (for example, hydrochloric acid, sodium hydroxide, phosphate, citrate, and sodium cyanide); tonicity agents (for example, sodium bisulfite, sodium sulfite, glycerin, and propylene glycol); and chelating agents (for example, ascorbic acid, sodium edetate, and citric acid).

Depending on the target tissue, it may be appropriate to formulate the pharmaceutical composition for sustained or timed release. Oral timed release formulations may include a mixture of isomeric variants, binding agents, or coatings. Injectable time release formulations may include the active agent in combination with a binding agent, encapsulating agent, or microparticle.

For treatment of joint diseases such as osteoarthritis, the senolytic combinations of the invention are typically, for example, formulated for intra-articular administration. For treatment of eye disease such as glaucoma, diabetic retinopathy or age-related macular degeneration (AMD), the senolytic combinations of the invention may be formulated, for example, for intravitreal or intracameral administration. For treatment of lung diseases, the senolytic combinations of the invention may be formulated, for example, as an aerosol for intratracheal administration. For treatment of cardiovascular diseases or the treatment of hepatic diseases, the senolytic combinations of the invention may be formulated, for example, for systemic administration, which can take place via enteral administration (absorption of the drug through the gastrointestinal tract) or parenteral administration (generally injection, infusion, or implantation).

This invention provides commercial products that are kits that enclose unit doses of one or more of the agents or compositions described in this disclosure. Such kits typically comprise a pharmaceutical preparation in one or more containers. The preparations may be provided as one or more unit doses (either combined or separate). The kit may contain a device such as a syringe for administration of the senolytic agent or composition in or around the target tissue of a subject in need thereof. The product may also contain or be accompanied by an informational package insert describing the use and attendant benefits of the senolytic drugs in treating the senescence-associated disease or disorder, and optionally a device for delivery of the senolytic drugs.

### Treatment Design

Senescent cells accumulate with age, which is why conditions mediated by senescent cells occur more frequently in older adults. In addition, different types of stress on tissues may promote the emergence of senescent cells and the phenotype they express. Cell stressors include oxidative stress, metabolic stress, DNA damage (for example, as a result of environmental ultraviolet light exposure or genetic disorder), oncogene activation, and telomere shortening (resulting, for example, from hyperproliferation). Tissues that are subject to such stressors may have a higher prevalence of senescent cells, which in turn may lead to presentation of certain conditions at an earlier age, or in a more severe form. An inheritable susceptibility to certain conditions suggests that the accumulation of disease-mediating senescent cells may directly or indirectly be influenced by genetic components, which can lead to earlier presentation.

To treat a particular senescence-associated disease or disorder with a combination senolytic therapy according to this invention, the therapeutic regimen will depend on the location of the senescent cells, and the pathophysiology of the disease.

### Senescence-Associated Disease or Disorders Suitable for Treatment

The Bcl and Mcl-1 inhibitors of the invention can be used for prevention or treatment of various senescence-associated disease or disorders. Such conditions will typically (although not necessarily) be characterized by an overabundance of senescent cells (such as cells expressing, for example, p16 and other senescence markers, and/or secretion of SASPs) in or around the site of the disease or disorder, or an overabundance of expression of p16 and other senescence markers, and/or secretion of SASPs, in comparison with the number of such cells or the level of such expression in unaffected cells and/or tissues. Non-limiting examples of senescence-associated diseases or disorders include the treatment of osteoarthritis, eye disease, lung disease, atherosclerosis, liver disease and kidney disease, as illustrated in the following sections.

### Treatment of Osteoarthritis

The senolytic combinations of the invention can be developed for treating osteoarthritis. Similarly, the senolytic combinations of the invention can be developed for selectively eliminating senescent cells in or around a joint of a subject in need thereof, including but not limited to a joint affected by osteoarthritis.

Osteoarthritis degenerative joint disease is characterized by fibrillation of the cartilage at sites of high mechanical stress, bone sclerosis, and thickening of the synovium and the joint capsule. Fibrillation is a local surface disorganization involving splitting of the superficial layers of the cartilage. The early splitting is tangential with the cartilage surface, following the axes of the predominant collagen bundles. Collagen within the cartilage becomes disorganized, and proteoglycans are lost from the cartilage surface. In the absence of protective and lubricating effects of proteoglycans in a joint, collagen fibers become susceptible to degradation, and mechanical destruction ensues. Predisposing risk factors for developing osteoarthritis include increasing age, obesity, previous joint injury, overuse of the joint, weak thigh muscles, and genetics. Symptoms of osteoarthritis include sore or stiff joints, particularly the hips, knees, and lower back, after inactivity or overuse; stiffness after resting that goes away after movement; and pain that is worse after activity or toward the end of the day.

The senolytic combinations of the invention can be used to reduce or inhibit loss or erosion of proteoglycan layers in a joint, reduces inflammation in the affected joint, and promotes, stimulates, enhances, or induces production of collagen, for example, type 2 collagen. The senolytic combinations of the invention may cause a reduction in the amount, or level, of inflammatory cytokines, such as IL-6, produced in a joint and inflammation is reduced. The senolytic combinations of the invention can be used for treating osteoarthritis and/or inducing collagen, for example, Type 2 collagen, production in the joint of a subject. The senolytic combinations of the invention also can be used for decreasing, inhibiting, or reducing production of metalloproteinase 13 (MMP-13), which degrades collagen in a joint, and for restoring proteoglycan layer or inhibiting loss and/or degradation of the proteoglycan layer. Treatment with the senolytic combinations of the invention may also reduce the likelihood of, inhibits, or decreases erosion, or slows erosion of the bone. The senolytic combinations of the invention may be administered directly to an osteoarthritic joint, for example, intra-articularly, topically, transdermally, intradermally, or subcutaneously. The senolytic combinations of the invention may also restore, improve, or inhibit deterioration of strength of a join, and reduce joint pain.

### Treatment of Ophthalmic Conditions

The senolytic combinations of the invention can be used for preventing or treating an ophthalmic condition in a subject in need thereof by removing senescent cells in or around an eye of the subject, whereby at least one sign or symptom of the disease is decreased in severity. Such conditions include both back-of-the-eye diseases, and front-of-the-eye diseases. Similarly, the senolytic combinations of the invention can be developed for selectively eliminating senescent cells in or around ocular tissue in a subject in need thereof.

Diseases of the eye that can be treated according to this invention include presbyopia, macular degeneration (including wet or dry AMD), diabetic retinopathy, and glaucoma.

Macular degeneration is a neurodegenerative condition that can be characterized as a back-of-the-eye disease, it causes the loss of photoreceptor cells in the central part of retina, called the macula. Macular degeneration can be dry or wet. The dry form is more common than the wet, with about 90% of age-related macular degeneration (AMD) patients diagnosed with the dry form. The wet form of the disease can lead to more serious vision loss. Age and certain genetic factors and environmental factors can be risk factors for developing AMD. Environmental factors include, for example, omega-3 fatty acids intake, estrogen exposure, and increased serum levels of vitamin D. Genetic risk factors can include, for example, reduced ocular Dicer1 levels, and decreased micro RNAs, and DICER1 ablation.

Dry AMD is associated with atrophy of the retinal pigment epithelium (RPE) layer, which causes loss of photoreceptor cells. The dry form of AMD can result from aging and thinning of macular tissues and from deposition of pigment in the macula. With wet AMD, new blood vessels can grow beneath the retina and leak blood and fluid. Abnormally leaky choroidal neovascularization can cause the retinal cells to die, creating blind spots in central vision. Different forms of macular degeneration can also occur in younger patients. Non-age related etiology can be linked to, for example, heredity, diabetes, nutritional deficits, head injury, or infection.

The formation of exudates, or “drusen,” underneath the Bruch's membrane of the macula is can be a physical sign that macular degeneration can develop. Symptoms of macular degeneration include, for example, perceived distortion of straight lines and, in some cases, the center of vision appears more distorted than the rest of a scene; a dark, blurry area or “white-out” appears in the center of vision; or color perception changes or diminishes.

Another back-of-the-eye disease is diabetic retinopathy (DR). According to Wikipedia, the first stage of DR is non-proliferative, and typically has no substantial symptoms or signs. NPDR is detectable by fundus photography, in which microaneurysms (microscopic blood-filled bulges in the artery walls) can be seen. If there is reduced vision, fluorescein angiography can be done to see the back of the eye. Narrowing or blocked retinal blood vessels can be seen clearly and this is called retinal ischemia (lack of blood flow). Macular edema in which blood vessels leak their contents into the macular region can occur at any stage of NPDR. The symptoms of macular edema are blurred vision and darkened or distorted images that are not the same in both eyes. Ten percent (10%) of diabetic patients will have vision loss related to macular edema. Optical Coherence Tomography can show the areas of retinal thickening (due to fluid accumulation) of macular edema.

In the second stage of DR, abnormal new blood vessels (neovascularisation) form at the back of the eye as part of proliferative diabetic retinopathy (PDR); these can burst and bleed (vitreous hemorrhage) and blur the vision, because these new blood vessels are fragile. The first time this bleeding occurs, it may not be very severe. In most cases, it will leave just a few specks of blood, or spots floating in a person's visual field, though the spots often go away after few hours. These spots are often followed within a few days or weeks by a much greater leakage of blood, which blurs the vision. In extreme cases, a person may only be able to tell light from dark in that eye. It may take the blood anywhere from a few days to months or even years to clear from the inside of the eye, and in some cases the blood will not clear. These types of large hemorrhages tend to happen more than once, often during sleep.

On funduscopic exam, a doctor will see cotton wool spots, flame hemorrhages (similar lesions are also caused by the alpha-toxin of Clostridium novyi), and dot-blot hemorrhages.

Presbyopia is an age-related condition where the eye exhibits a progressively diminished ability to focus on near objects as the speed and amplitude of accommodation of a normal eye decreases with advancing age. Loss of elasticity of the crystalline lens and loss of contractility of the ciliary muscles can cause presbyopia. Age-related changes in the mechanical properties of the anterior lens capsule and posterior lens capsule suggest that the mechanical strength of the posterior lens capsule decreases significantly with age.

The laminated structure of the capsule of the eye also changes and can result, at least in part, from a change in the composition of the tissue. The major structural component of the lens capsule is basement membrane type IV collagen that is organized into a three-dimensional molecular network. Type IV collagen is composed of six homologous a chains (α1-6) that associate into heterotrimeric collagen IV protomers with each comprising a specific chain combination of α112, α345, or α556. Protomers share structural similarities of a triple-helical collagenous domain with the triplet peptide sequence of Gly-X-Y, ending in a globular C-terminal region termed the non-collagenous 1 (NC1) domain. The N-termini are composed of a helical domain termed the 7S domain, which is also involved in protomer-protomer interactions.

Collagen IV can influence cellular function and tissue stabilization. Posterior capsule opacification (PCO) develops as a complication in approximately 20-40% of patients in subsequent years after cataract surgery. PCO results from proliferation and activity of residual lens epithelial cells along the posterior capsule in a response akin to wound healing. Growth factors, such as fibroblast growth factor, transforming growth factor β, epidermal growth factor, hepatocyte growth factor, insulin-like growth factor, and interleukins IL-1 and IL-6, can also promote epithelial cell migration. In vitro, collagen IV can promote adherence of lens epithelial cells. Adhesion of the collagen IV, fibronectin, and laminin to the intraocular lens can inhibit cell migration and can reduce the risk of PCO.

Compounds provided by this disclosure can slow the disorganization of the type IV collagen network, decrease or inhibit epithelial cell migration and can also delay the onset of presbyopia or decrease or slow the progressive severity of the condition. They can also be useful for post-cataract surgery to reduce the likelihood of occurrence of PCO.

Another condition treatable according to the methods of the invention is glaucoma. Normally, clear fluid flows into and out of the front part of the eye, known as the anterior chamber. In individuals who have open/wide-angle glaucoma, the clear fluid drains too slowly, leading to increased pressure within the eye. If left untreated, the high pressure in the eye can subsequently damage the optic nerve and can lead to complete blindness. The loss of peripheral vision is caused by the death of ganglion cells in the retina. The effect of a therapy on inhibiting progression of glaucoma can be monitored by automated perimetry, gonioscopy, imaging technology, scanning laser tomography, HRT3, laser polarimetry, GDX, ocular coherence tomography, ophthalmoscopy, and pachymeter measurements that determine central corneal thickness.

Ophthalmic conditions treatable with the senolytic combinations of the invention include ischemic or vascular conditions, such as diabetic retinopathy, glaucomatous retinopathy, ischemic arteritic optic neuropathies, and vascular diseases characterized by arterial and venous occlusion, retinopathy of prematurity and sickle cell retinopathy.

Ophthalmic conditions treatable with the senolytic combinations of the invention include degenerative conditions, such as dermatochalasis, ptosis, keratitis sicca, Fuch's corneal dystrophy, presbyopia, cataract, wet age related macular degeneration (wet AMD), dry age related macular degeneration (dry AMD); degenerative vitreous disorders, including vitreomacular traction (VMT) syndrome, macular hole, epiretinal membrane (ERM), retinal tears, retinal detachment, and proliferative vitreoretinopathy (PVR).

Ophthalmic conditions treatable with the senolytic combinations of the invention include genetic conditions, such as retinitis pigmentosa, Stargardt disease, Best disease and Leber's hereditary optic neuropathy (LHON). Ophthalmic conditions treatable with the senolytic combinations of the invention include conditions caused by a bacterial, fungal, or virus infection. These include conditions caused or provoked by an etiologic agent such as herpes zoster varicella (HZV), herpes simplex, cytomegalovirus (CMV), and human immunodeficiency virus (HIV).

Ophthalmic conditions treatable with the senolytic combinations of the invention include inflammatory conditions, such as punctate choroiditis (PIC), multifocal choroiditis (MIC) and serpiginous choroidopathy. Ophthalmic conditions treatable with the senolytic combinations of the invention also include iatrogenic conditions, such as a post-vitrectomy cataract and radiation retinopathy.

### Treatment of Pulmonary Conditions

The senolytic combinations of the invention can be developed for treating pulmonary disease in accordance with this invention. Similarly, the senolytic combinations of the invention can be developed for selectively eliminating senescent cells in or around a lung of a subject in need thereof. Pulmonary conditions that can be treated according to this invention include idiopathic pulmonary fibrosis (IPF), chronic obstructive pulmonary disease (COPD), asthma, cystic fibrosis, bronchiectasis, and emphysema.

COPD is a lung disease defined by persistently poor airflow resulting from the breakdown of lung tissue, emphysema, and the dysfunction of the small airways, obstructive bronchiolitis. Primary symptoms of COPD include shortness of breath, wheezing, chest tightness, chronic cough, and excess sputum production. Elastase from cigarette smoke-activated neutrophils and macrophages can disintegrate the extracellular matrix of alveolar structures, resulting in enlarged air spaces and loss of respiratory capacity. COPD can be caused by, for example, tobacco smoke, cigarette smoke, cigar smoke, secondhand smoke, pipe smoke, occupational exposure, exposure to dust, smoke, fumes, and pollution, occurring over decades thereby implicating aging as a risk factor for developing COPD.

The processes that cause lung damage include, for example, oxidative stress produced by the high concentrations of free radicals in tobacco smoke, cytokine release due to the inflammatory response to irritants in the airway, and impairment of anti-protease enzymes by tobacco smoke and free radicals, allowing proteases to damage the lungs. Genetic susceptibility can also contribute to the disease. In about 1% percent of people with COPD, the disease results from a genetic disorder that causes low level production of alpha-1-antitrypsin in the liver. Alpha-1-antitrypsin is normally secreted into the bloodstream to help protect the lungs.

Pulmonary fibrosis is a chronic and progressive lung disease characterized by stiffening and scarring of the lung, which can lead to respiratory failure, lung cancer, and heart failure. Fibrosis is associated with repair of epithelium. Fibroblasts are activated, production of extracellular matrix proteins is increased, and transdifferentiation to contractile myofibroblasts contribute to wound contraction. A provisional matrix plugs the injured epithelium and provides a scaffold for epithelial cell migration, involving an epithelial-mesenchymal transition (EMT). Blood loss associated with epithelial injury induces platelet activation, production of growth factors, and an acute inflammatory response. Normally, the epithelial barrier heals and the inflammatory response resolves. However, in fibrotic disease the fibroblast response continues, resulting in unresolved wound healing. Formation of fibroblastic foci is a feature of the disease, reflecting locations of ongoing fibrogenesis.

Subjects at risk of developing pulmonary fibrosis include, for example, those exposed to environmental or occupational pollutants, such as asbestosis and silicosis; those who smoke cigarettes; those who have a connective tissue diseases such as RA, SLE, scleroderma, sarcoidosis, or Wegener's granulomatosis; those who have infections; those who take certain medications, including, for example, amiodarone, bleomycin, busufan, methotrexate, and nitrofurantoin; those subject to radiation therapy to the chest; and those whose family member have pulmonary fibrosis.

Symptoms of COPD can include any one of shortness of breath, wheezing, chest tightness, having to clear one's throat first thing in the morning because of excess mucus in the lungs, a chronic cough that produces sputum that can be clear, white, yellow or greenish, cyanosis, frequent respiratory infections, lack of energy, and unintended weight loss. Subjects with COPD can also experience exacerbations, during which symptoms worsen and persist for days or longer. Symptoms of pulmonary fibrosis include, for example, shortness of breath, particularly during exercise; dry, hacking cough; fast, shallow breathing; gradual, unintended weight loss; fatigue; aching joints and muscles; and clubbing of the fingers or toes.

Other pulmonary conditions that can be treated by using a senolytic combination of the invention include emphysema, asthma, bronchiectasis, and cystic fibrosis. Pulmonary diseases can also be exacerbated by tobacco smoke, occupational exposure to dust, smoke, or fumes, infection, or pollutants that contribute to inflammation.

Bronchiectasis can result from damage to the airways that causes them to widen and become flabby and scarred. Bronchiectasis can be caused by a medical condition that injures the airway walls or inhibits the airways from clearing mucus. Examples of such conditions include cystic fibrosis and primary ciliary dyskinesia (PCD). When only one part of the lung is affected, the disorder can be caused by a blockage rather than a medical condition.

The methods of this invention for treating or reducing the likelihood of a pulmonary condition can also be used for treating a subject who is aging and has loss of pulmonary function, or degeneration of pulmonary tissue. The respiratory system can undergo various anatomical, physiological and immunological changes with age. The structural changes include chest wall and thoracic spine deformities that can impair the total respiratory system compliance resulting in increased effort to breathe. The respiratory system undergoes structural, physiological, and immunological changes with age. An increased proportion of neutrophils and lower percentage of macrophages can be found in bronchoalveolar lavage (BAL) of older adults compared with younger adults. Persistent low grade inflammation in the lower respiratory tract can cause proteolytic and oxidant-mediated injury to the lung matrix resulting in loss of alveolar unit and impaired gas exchange across the alveolar membrane seen with aging. Sustained inflammation of the lower respiratory tract can predispose older adults to increased susceptibility to toxic environmental exposure and accelerated lung function decline. Oxidative stress exacerbates inflammation during aging. Alterations in redox balance and increased oxidative stress during aging precipitate the expression of cytokines, chemokines, and adhesion molecules, and enzymes. Constitutive activation and recruitment of macrophages, T cells, and mast cells foster release of proteases leading to extracellular matrix degradation, cell death, remodeling, and other events that can cause tissue and organ damage during chronic inflammation.

Effects of treatments of the invention can be determined using techniques that evaluate mechanical functioning of the lung, for example, techniques that measure lung capacitance, elastance, and airway hypersensitivity can be performed. To determine lung function and to monitor lung function throughout treatment, any one of numerous measurements can be obtained, for example, expiratory reserve volume (ERV), forced vital capacity (FVC), forced expiratory volume (FEV) (e.g., FEV in one second, FEV1), FEV1/FEV ratio, forced expiratory flow 25% to 75%, and maximum voluntary ventilation (MVV), peak expiratory flow (PEF), slow vital capacity (SVC). Total lung volumes include total lung capacity (TLC), vital capacity (VC), residual volume (RV), and functional residual capacity (FRC). Gas exchange across alveolar capillary membrane can be measured using diffusion capacity for carbon monoxide (DLCO).

Peripheral capillary oxygen saturation (SpO₂) can also be measured; normal oxygen levels are typically between 95% and 100%. An SpO₂ level below 90% suggests the subject has hypoxemia. Values below 80% are considered critical and require intervention to maintain brain and cardiac function and avoid cardiac or respiratory arrest.

### Treatment of Hepatic Conditions

Mortality rates for chronic liver disease are rising, and pose a serious burden on health care systems worldwide. Chronic liver disease results in liver inflammation. This in turn leads to loss of functional hepatocytes, fibrosis, and ultimately cirrhosis and increased risk for liver cancer. In late-stage liver disease, decompensation of the liver leads to mortality rates of up to 85% within 5 years, unless the patent is fortunate above to have a liver transplant.

Liver disease can arise from a diversity of causes including infection, genetic disorders, dietary lifestyle choices and substance abuse. These include infection with Hepatitis B or C viruses (viral hepatitis), excessive alcohol intake (alcoholic hepatitis), autoimmune-associated disease (primary sclerosing cholangitis, primary biliary cholangitis, autoimmune hepatitis), and genetic disorders such as α1 antitrypsin deficiency and hemochromatosis (arising from specific mutations in susceptibility genes A1AT and HFE, respectively). Some diseases arise from a combination of metabolic disease, diet and genetic predisposition. These include non-alcoholic fatty liver disease (NAFLD) and non-alcoholic steatohepatitis (NASH). Any of these conditions can lead to severe chronic liver disease, and ultimately hepatic failure or liver transplant.

This invention provides a new approach to treating liver disease by eliminating senescent cells that reside in or around the site of the disease pathophysiology. Senescent cells having particular features have been identified as suitable targets for pharmacological intervention. Removal of senescent cells from affected sites using small molecule agents that specifically target senescent cells can help ameliorate signs and symptoms of liver disease, and prevent progression to more severe stages of the disease.

As a non-limiting example, screening candidate senolytic combinations of the invention for selective elimination of p16-expressing hepatocytes or cholangiocytes can be performed as follows. In chronic liver diseases, both hepatocytes and cholangiocytes have been implicated as contributing to populations of p16-expressing senescent cells. To prepare p16 positive liver cells, cryopreserved human hepatocytes (obtained from SigmaAldrich®) or primary cryopreserved human cholangiocytes (obtained from Celprogen®) are seeded and plated in multiwell plates at densities appropriate for the number of wells desired to be used in the assay, which should be confined to 24, 96, or 384 wells. After cell seeding, cells are challenged with a small molecule compound that induces senescence, or with radiation. A dose-response time course of senescence induction can be used to assess the kinetics of expression of p21 and p16 during this process. To test potential senolytic agents for selective elimination of senescent hepatocytes or cholangiocytes, various doses of senolytic compounds are compared with vehicle treatments to assess the number of p16 and p21 expressing cells that survive after a defined period of treatment (typically 1 to 5 days). Assessment of total cell viability in this assay is performed using Cell-Titer Glow™ assay (Promega®) to control for compounds that induce non-specific cell death. Vehicle and senolytic compound treatment groups are compared by quantitation of cells positive for p16, p21, both or neither as quantified using high content microscopic immunocytochemical methods with antibodies against p16, p21 (Dako®) and DAPI nuclear stain to determine selectivity indexes for p16, p21, and p16/p21.

For non-limiting in vivo hepatic models, the STAM™ mouse model of NASH recapitulates the histological progression of NASH observed in human patients and includes similar effects on liver function and development of hepatocellular carcinoma (Saito, T et al., Intern Med. 2007; 46(2):101-3, and Saito, K et al., Physiol Res. 2017 Nov. 24; 66(5):791-799). After birth, animals are injected with streptozotocin (STZ) to ablate pancreatic beta-cells and induce metabolic disease in the form of insufficient insulin formation. The animals are then put on a high fat diet (HFD) to induce sever metabolic disease and NAFLD, which progresses in a predictable time course to NASH and ultimately to hepatocellular carcinoma (HCC) by 20 weeks.

There is an increase in the burden and distribution of senescent cells in the mouse STAM™ model of NASH that mirrors what is observed in samples from human patients diagnosed with NASH. Elevation in the number of p16-positive cells occurs by 8 weeks after HFD is initiate. This supports the hypothesis that p16-positive senescent cells may have an immediate influence on the progression of disease, prior to the development of cirrhosis and hepatocellular carcinoma (HCC).

In accordance with this model, treatment of the mice with a senolytic agent can begin 5 to 12 weeks after HFD is initiated. The candidate senolytic combinations of the invention are dosed systemically, enterally or parenterally as needed. A positive end-point may be shown by reduction of p16-positive cells in liver parenchyma any time between 6 and 20 weeks after initiation. A positive end point may also be shown by reduction of fibrosis, as determined by Sirius red or trichome histology at 20 weeks. Reduction of either of these markers may correlate with a reduced likelihood of HCC development in the model, which can be assessed by quantitation of tumor burden and nodule formation at 20 weeks.

The senolytic compounds, conjugates, and formulations of this invention can be administered for the treatment or prevention of liver disease at any stage. It is often desirable to assess patients that will progress to an acute liver insult such as acute hepatitis, NAFLD, or NASH, to cirrhosis and ultimately to liver failure. Assessing liver function can be done according to standard tests for liver function, including serum markers and characteristic liver histopathology. Candidate patients can be graded for disease progression prior to end stage disease as described by Eddowes et al., Aliment Pharmacol Ther. 2018 March; 47(5):631-644.

Efficacy of senolytic treatment can be measured by changes in circulating liver enzyme levels (aspartate transaminase (AST) and alanine transaminase (ALT)), the five-year risk score for requiring a liver transplant, development of HCC, and progression-free survival.

### Treatment of Atherosclerosis

Atherosclerosis is characterized by patchy intimal plaques (atheromas) that encroach on the lumen of medium-sized and large arteries; the plaques contain lipids, inflammatory cells, smooth muscle cells, and connective tissue. Atherosclerosis can affect large and medium-sized arteries, including the coronary, carotid, and cerebral arteries, the aorta and its branches, and major arteries of the extremities.

Atherosclerosis is a syndrome affecting arterial blood vessels due in significant part to a chronic inflammatory response of white blood cells in the walls of arteries. This is promoted by low-density lipoproteins (LDL, plasma proteins that carry cholesterol and triglycerides) in the absence of adequate removal of fats and cholesterol from macrophages by functional high-density lipoproteins (HDL). The earliest visible lesion of atherosclerosis is the “fatty streak,” which is an accumulation of lipid-laden foam cells in the intimal layer of the artery. The hallmark of atherosclerosis is atherosclerotic plaque, which is an evolution of the fatty streak and has three major components: lipids (e.g., cholesterol and triglycerides); inflammatory cells and smooth muscle cells; and a connective tissue matrix that may contain thrombi in various stages of organization and calcium deposits.

Within the outer-most and oldest plaque, calcium and other crystallized components (e.g., microcalcification) from dead cells can be found. Microcalcification and properties related thereto are also thought to contribute to plaque instability by increasing plaque stress. Fatty streaks reduce the elasticity of the artery walls, but may not affect blood flow for years because the artery muscular wall accommodates by enlarging at the locations of plaque. Lipid-rich atheromas are at increased risk for plaque rupture and thrombosis. Reports have found that of all plaque components, the lipid core exhibits the highest thrombogenic activity. Within major arteries in advanced disease, the wall stiffening may also eventually increase pulse pressure.

A vulnerable plaque that may lead to a thrombotic event (stroke or myocardial infarction (MI), commonly known as a heart attack) and is sometimes described as a large, soft lipid pool covered by a thin fibrous cap. An advanced characteristic feature of advance atherosclerotic plaque is irregular thickening of the arterial intima by inflammatory cells, extracellular lipid (atheroma) and fibrous tissue (sclerosis. Fibrous cap formation is believed to occur from the migration and proliferation of vascular smooth muscle cells and from matrix deposition. A thin fibrous cap contributes instability of the plaque and to increased risk for rupture.

The methods and senolytic combinations according to this invention may have any one or more of the following effects: inhibit formation, increase stability, increase fibrous cap thickness, decrease lipid concentration of atherosclerotic plaques, inhibit calcium deposition in blood vessels, preventing or inhibiting progression of angina, and thus decreasing the risk of an infarction.

### Definitions

A “senescent cell” is generally thought to be derived from a cell type that typically replicates, but as a result of aging or other event that causes a change in cell state, can no longer replicate. For the purpose of practicing aspects of this invention, senescent cells can be identified as, for example, expressing p16, or at least one marker selected from p16, senescence-associated β-galactosidase, and lipofuscin; sometimes two or more of these markers, and other markers of SASP such as, but not limited to, interleukin 6, and inflammatory, angiogenic and extracellular matrix modifying proteins.

A “senescence-associated”, “senescence-related” or “age-related” disease, disorder, or condition as referred to in this disclosure is a physiological condition that is caused or mediated in part by senescent cells, which may be induced by multiple etiologic factors including age, DNA damage, oxidative stress, genetic defects, etc. Lists of senescence associated disorders that can potentially be treated or managed using the methods and products taught in this disclosure.

A compound, composition or agent is typically referred to as “senolytic” if it eliminates senescent cells, compared with replicative cells of the same tissue type, or quiescent cells lacking SASP markers. Alternatively, or in addition, the methods and senolytic combinations of the invention may effectively be used according to this invention if it decreases the release of pathological soluble factors or mediators as part of the senescence associated secretory phenotype (SASP) that play a role in the initial presentation or ongoing pathology of a condition, or inhibit its resolution. In this respect, the term “senolytic” is exemplary, with the understanding that compounds that work primarily by inhibiting rather than eliminating senescent cells (senescent cell inhibitors) can be used in a similar fashion with ensuing benefits.

Selective removal or “elimination” of senescent cells from a mixed cell population or tissue does not necessarily require that all cells bearing a senescence phenotype in a target tissue or organ be removed: only that the proportion of senescent cells initially in the tissue that remain after treatment is substantially lower than the proportion of non-senescent cells initially in the tissue that remain after the treatment.

The terms “disease,” “disorder,” or “condition” are used interchangeable to refer to any condition of a human or animal body that has signs, symptoms, and/or phenotypical features that are in some respects undesirable to the subject, for which the subject desires is deemed to be worthy of treatment according to this invention.

Successful “treatment” of a senescence-associated disease or disorder, according to this invention, may have any effect that is beneficial to the subject being treated. This includes decreasing the severity, duration, or progression of a senescence-associated disease or disorder, or of any adverse signs or symptoms resulting therefrom. In some circumstances, senolytic agents can also be used to prevent or inhibit presentation of a senescence-associated disease or disorder for which a subject is susceptible, for example, because of an inherited susceptibility or because of medical history.

A “therapeutically effective amount” is an amount of a compound of the invention that (i) treats the particular senescence-associated disease or disorder, (ii) attenuates, ameliorates, or eliminates one or more symptoms of the particular senescence-associated disease or disorder, (iii) prevents or delays the onset of one or more symptoms of the particular senescence-associated disease or disorder described herein, (iv) prevents or delays progression of the particular senescence-associated disease or disorder, or (v) at least partially reverses damage caused by the senescence-associated disease or disorder prior to treatment.

“Enhancement of senolytic activity”, according to this invention, means the ability for the combination therapies of the invention to demonstrate a synergistic senolytic activity on senescent cells, which is more than an additive senolytic activity of each individual senolytic compound by itself. This can be calculated by using, for example, the Zero interaction potency (ZIP) model, described herein and in Example 7. The senolytic activity for any individual senolytic compound and for any senolytic combination may be measured by, for example, a dose-response assay as described in Example 7.

A “phosphorylated” form of a compound is a compound in which one or more —OH or —COOH groups have been substituted with a phosphate group which is either —OPO₃H₂ or —CₙPO₃H₂ (where n is 1 to 4). This includes phosphorylated forms that act as prodrugs by including a phosphate group that may be removed in vivo (for example, by enzymolysis). A non-phosphorylated or dephosphorylated form has no such group.

“Prodrug” refers to a derivative of an active agent that requires a transformation within the body to release the active agent. The transformation can be an enzymatic transformation. Prodrugs are frequently, although not necessarily, pharmacologically inactive until converted to the active agent.

Unless otherwise stated or required, each of the compound structures referred to in the invention include conjugate acids and bases having the same structure, crystalline and amorphous forms of those compounds, pharmaceutically acceptable salts, and prodrugs. This includes, for example, polymorphs, solvates, hydrates, unsolvated polymorphs (including anhydrates), and phosphorylated and unphosphorylated forms of the compounds.

The term “alkenyl” refers to a monovalent linear or branched chain group of one to twelve carbon atoms, and such as 1 to 6 carbon atoms, or 1 to 5, or 1 to 4, or 1 to 3 carbon atoms, derived from a straight or branched chain hydrocarbon (hydrocarbyl) containing at least one carbon-carbon double bond.

The term “alkoxy” refers to an alkyl group attached to the parent molecular moiety through an oxygen atom.

The term “alkoxyalkyl” refers to an alkoxy group attached to the parent molecular moiety through an alkyl group.

The term “alkoxycarbonyl” refers to an alkoxy group attached to the parent molecular moiety through a carbonyl group.

The term “alkoxycarbonyl” refers to an alkoxy group attached to the parent molecular moiety through a carbonyl group.

The term “alkoxycarbonylalkyl” refers to an alkoxycarbonyl group attached to the parent molecular moiety through an alkyl group.

The term “alkyl” refers to a monovalent saturated aliphatic hydrocarbyl group having from 1 to 12 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “alkylaminosulfonyl” refers to an alkylamino group attached to the parent molecular moiety through a sulfonyl group.

The term “alkylsulfanyl” refers to an alkyl group attached to the parent molecular moiety through a sulfur atom (—S—).

The term “alkylsulfinyl” refers to an alkyl group attached to the parent molecular moiety through a sulfinyl group (—SO—).

The term “alkylsulfonyl” refers to an alkyl group attached to the parent molecular moiety through a sulfonyl group (—SO₂—).

The term “alkylsulfonylalkyl” refers to an alkylsulfonyl group attached to the parent molecular moiety through an alkyl group.

The term “alkylsulfonylalkyl” refers to an alkylsulfonyl group attached to the parent molecular moiety through an amino group (—NR^{a}—) wherein R^{a} is hydrogen, alkanoyl, alkenyl, alkoxyalkyl, alkoxyalkoxyalkyl, alkoxycarbonyl, alkyl, alkylaminoalkyl, alkylaminocarbonylalkyl, aryl, arylalkyl, cycloalkyl, (cycloalkyl)alkyl, cycloalkylcarbonyl, haloalkanoyl, haloalkyl, (heterocycle)alkyl, heterocyclecarbonyl, hydroxyalkyl, a nitrogen protecting group, —C(NH)NH₂, or —C(O)NR^{c}R^{d}, where R^{c} and R^{d} are hydrogen, alkyl, aryl, heteroaryl, carbocycle or heterocycle.

The term “alkynyl” refers to a straight or branched chain hydrocarbyl group of one to twelve carbon atoms, and such as 1 to 6 carbon atoms, or 1 to 5, or 1 to 4, or 1 to 3 carbon atoms, containing at least one carbon-carbon triple bond.

The term “amino” refers to —NR^{a}R^{b}, wherein R^{a} and R^{b} are hydrogen, alkanoyl, alkenyl, alkoxyalkyl, alkoxyalkoxyalkyl, alkoxycarbonyl, alkyl, alkylaminoalkyl, alkylaminocarbonylalkyl, aryl, arylalkyl, cycloalkyl, (cycloalkyl)alkyl, cycloalkylcarbonyl, haloalkanoyl, haloalkyl, (heterocycle)alkyl, heterocyclecarbonyl, hydroxyalkyl, a nitrogen protecting group, —C(NH)NH₂, or —C(O)NR^{c}R^{d}, where R^{c} and R^{d} are hydrogen, alkyl, aryl, heteroaryl, carbocycle or heterocycle; wherein the aryl; the aryl part of the arylalkyl; the cycloalkyl; the cycloalkyl part of the (cycloalkyl)alkyl and the cycloalkylcarbonyl; and the heterocycle part of the (heterocycle)alkyl and the heterocyclecarbonyl can be optionally substituted with one, two, three, four, or five substituents independently selected from the group consisting of alkanoyl, alkoxy, alkyl, cyano, halo, haloalkoxy, haloalkyl, hydroxy, and nitro.

The term “aminosulfonyl” refers to an amino group attached to the parent molecular moiety through a sulfonyl group.

The terms “Aryl” or “Ar” refer to a monovalent aromatic carbocyclic group of from 6 to 18 carbon atoms having a single ring (such as is present in a phenyl group) or a ring system having multiple condensed rings, e.g., a bicyclic fused ring system or a tricyclic fused ring system (examples of such aromatic ring systems include naphthyl, anthryl and indanyl), which condensed rings may or may not be aromatic, provided that the point of attachment is through an atom of an aromatic ring. This term includes, by way of example, phenyl and naphthyl. Bicyclic fused ring systems are exemplified by a phenyl group fused to a cycloalkyl group as defined herein, a cycloalkenyl group as defined herein, or another phenyl group. Tricyclic fused ring systems are exemplified by a bicyclic fused ring system fused to a cycloalkyl group as defined herein, a cycloalkenyl group as defined herein, or another phenyl group. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

The term “arylalkoxy” refers an aryl group attached to the parent molecular moiety through an alkoxy group.

The term “arylalkyl” refers an aryl group attached to the parent molecular moiety through an alkyl group.

The term “arylcycloalkenylalkyl” refers a bicyclic aryl-cycloalkenyl group attached to the parent molecular moiety through an alkyl group.

The term “arylheteroarylalkyl” refers a bicyclic aryl-heteroaryl group attached to the parent molecular moiety through an alkyl group.

The term “aryloxy” refers to an aryl group attached to the parent molecular moiety through an oxygen atom.

The term “aryloxyalkoxy” refers an aryloxy group attached to the parent molecular moiety through an alkoxy group.

The term “aryloxyalkyl” refers to an aryloxy group attached to the parent molecular moiety through an alkyl group.

The term “arylsulfanyl” refers to an aryl group attached to the parent molecular moiety through a sulfur atom (—S—).

The term “arylsulfanylalkoxy” refers to an arylsulfanyl group attached to the parent molecular moiety through an alkoxy group.

The term “arylsulfanylalkyl” refers to an arylsulfanyl group attached to the parent molecular moiety through an alkyl group.

The term “arylsulfinyl” refers to an aryl group attached to the parent molecular moiety through a sulfinyl group (—SO—).

The term “arylsulfinylalkyl” refers to an arylsulfinyl group attached to the parent molecular moiety through an alkyl group.

The term “arylsulfonyl” refers to an aryl group attached to the parent molecular moiety through a sulfonyl group (—SO₂—).

The term “arylsulfonylalkyl” refers to an arylsulfonyl group attached to the parent molecular moiety through an alkyl group.

The term “biaryl”, unless indicated otherwise, refers to a group including two aryl rings linked via a single covalent bond.

The term “biarylalkyl” refers to a biaryl group attached to the parent molecular moiety through an alkyl group.

The term C₁₋ₙalkyl linker where n is an integer of 1 to 100, e.g., n is 2, 3, 4, 5, 6, or more, refers to a divalent alkyl linker that connects two groups and has a backbone of “n” atoms in length. The divalent alkyl linker is optionally substituted.

The terms “carbocycle” and “carbocyclic” refer to a saturated or unsaturated group having a single ring or multiple condensed rings, including fused, bridged and spiro ring systems, and having from 3 to 20 ring carbon atoms. In fused ring systems, one or more of the rings can be cycloalkyl or aryl, provided that the point of attachment is through the non-aromatic ring.

The term “carbonyloxy” refers to an alkanoyl group attached to the parent molecular moiety through an oxygen atom.

The terms “carboxyl”, “carboxy” or “carboxylate” refer to —CO₂H or salts thereof.

The term “carboxyalkyl” refers to a carboxy group attached to the parent molecular moiety through an alkyl group.

“Cyano” or “nitrile” refers to the group —CN.

The term “cycloalkenyl” refers to non-aromatic cyclic alkyl groups of from 3 to 10 carbon atoms having single or multiple rings and having at least one double bond and preferably from 1 to 2 double bonds.

The term “cycloalkenylalkyl” refers to a cycloalkenyl group attached to the parent molecular moiety through an alkyl group.

The term “cycloalkyl” refers to a saturated carbocyclic ring system having three to twelve carbon atoms and one to three rings including fused, bridged, and spiro ring systems. Examples of cycloalkyl groups include cyclopropyl, cyclobutyl, cyclopentyl, cyclooctyl, bicyclo(3.1.1)heptyl, adamantyl, and the like. The cycloalkyl groups of this invention can be optionally substituted with one, two, three, four, or five substituents independently selected from alkoxy, alkoxycarbonyl, alkyl, aminoalkyl, arylalkoxy, aryloxy, arylsulfanyl, halo, haloalkoxy, haloalkyl and hydroxy, where the aryl part of the arylalkoxy, the aryloxy, and the arylsulfanyl can be further optionally substituted with one, two, or three substituents independently selected from the group consisting of alkoxy, alkyl, halo, haloalkoxy, haloalkyl and hydroxy.

The term “cycloalkylalkoxy” refers to a cycloalkyl group attached to the parent molecular moiety through an alkoxy group.

The term “cycloalkylalkyl” refers to a cycloalkyl group attached to the parent molecular moiety through an alkyl group.

The term “cycloalkylcarbonyl” refers to a cycloalkyl group attached to the parent molecular moiety through a carbonyl group (—CO—).

The term “cycloalkyloxy” refers to a cycloalkyl group attached to the parent molecular moiety through an oxygen atom.

The term “dialkylamino” refers to —N(R)₂, wherein each R is alkyl.

The term “haloalkoxy” refers to an alkoxy group substituted by one, two, three, or four halogen atoms.

The term “haloalkyl” refers to an alkyl group substituted by one, two, three, or four halogen atoms.

“Heteroaryl” refers to an aromatic group of from 1 to 15 carbon atoms, such as from 1 to 10 carbon atoms, and 1 to 10 heteroatoms selected from oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, SO-heteroaryl, SO₂-alkyl, —SO₂-substituted alkyl, —SO₂-aryl and —SO₂-heteroaryl, and trihalomethyl.

The term “heteroarylalkyl” refers a heteroaryl group attached to the parent molecular moiety through an alkyl group.

The term “heteroarylarylalkyl” refers a bicyclic heteroaryl-aryl group attached to the parent molecular moiety through an alkyl group.

The term “arylcycloalkenylalkyl” refers a bicyclic heteroaryl-cycloalkenyl group attached to the parent molecular moiety through an alkyl group.

The term “heteroaryloxy” refers to a heteroaryl group attached to the parent molecular moiety through an oxygen atom.

The term “heteroaryloxyalkyl” refers a heteroaryloxy group attached to the parent molecular moiety through an alkyl group.

The term “heteroarylsulfanylalkyl” refers to a heteroarylsulfanyl group attached to the parent molecular moiety through an alkyl group.

The term “heteroarylsulfinylalkyl” refers to a heteroarylsulfinyl group attached to the parent molecular moiety through an alkyl group.

The term “heteroarylsulfonylalkyl” refers to a heteroarylsulfonyl group attached to the parent molecular moiety through an alkyl group.

The term “heterocycle-sulfanylalkyl” refers to a heterocycle group attached to the parent molecular moiety through a sulfonyl (—S—) and an alkyl group.

“Heterocycle” “heterocyclic” and “heterocyclyl” refer to a saturated or unsaturated group having a single ring or multiple condensed rings, including fused, bridged and spiro ring systems, and having from 3 to 20 ring atoms, including 1 to 10 heteroatoms. These ring heteroatoms are selected from nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties. When the heterocycle is saturated it may be referred to as a “heterocycloalkyl”.

The term “hydroxyalkyl” refers to a hydroxy group attached to the parent molecular moiety through an alkyl group.

The term “linker” or “linkage” refers to a linking moiety that connects at least two groups and has a backbone of 100 atoms or less in length between the at least two groups. A linker may be a covalent bond that connects two groups or a group having a backbone of between 1 and 100 atoms in length, for example a backbone of 1, 2, 3, 4, 5, 6, 8, 10, 12, 14, 16, 18 or 20 carbon atoms in length, where the linker may be linear, branched, cyclic or a single atom. A linker that is branched can connect three groups (i.e., trivalent). In certain cases, one, two, three, four or five or more carbon atoms of a linker backbone may be optionally substituted with a sulfur, nitrogen or oxygen heteroatom. The bonds between backbone atoms may be saturated or unsaturated, where usually not more than one, two, or three unsaturated bonds will be present in a linker backbone. The linker may include one or more substituent groups, for example an alkyl, aryl, heteroaryl or alkenyl group. A linker may include, without limitations, ethylene glycol or poly(ethylene glycol) units, ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heteroaryl, a heterocycle or a cycloalkyl group, where 2 or more atoms, e.g., 2, 3 or 4 atoms, of the cyclic group are included in the backbone. A linker may be cleavable or non-cleavable.

The term “monoalkylamino” refers to —NHR, where R is alkyl.

In addition to the disclosure herein, the term “substituted” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻ M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —O(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC (O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —O S(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “substituted alkoxy” refers to a substituted alkyl group attached to the parent molecular moiety through an oxygen atom.

The term “substituted alkyl” refers to an alkyl group where one or more carbon atoms in the alkyl chain have been optionally replaced with a heteroatom such as O—, N—, S—, —S(O)ₙ— (where n is 0 to 2), —NR— (where R is hydrogen or alkyl) and having from 1 to 5 substituents selected from alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, SO₂-heteroaryl and —NR^{a}R^{b}, where R^{a} and R^{b} may be the same or different and are chosen from hydrogen, optionally substituted alkyl, cycloalkyl, alkenyl, cycloalkenyl, alkynyl, aryl, heteroaryl and heterocyclic.

Except where otherwise stated or required, other terms used in the specification have their ordinary meaning.

### EXAMPLES

### Example 1: Inducing Senescence in Primary Human Cells

The ability to induce senescence in human primary cells in culture was performed to set up for in vitro experiments testing candidate senolytic combinations. Primary human small airway epithelial cells (SAEC) and human bronchial epithelial cells (HBEC) were obtained from Lonza®, ATCC®, and Promocell®. Cells were maintained and propagated at <75% confluency in Airway Epithelial Cell Growth Medium or Small Airway Epithelial Cell Growth Medium (Promocell®; Heidelberg, Germany) at 20% O2, 5% CO2, and ˜95% humidity. To make these primary cells senescent, x-ray irradiation was employed.

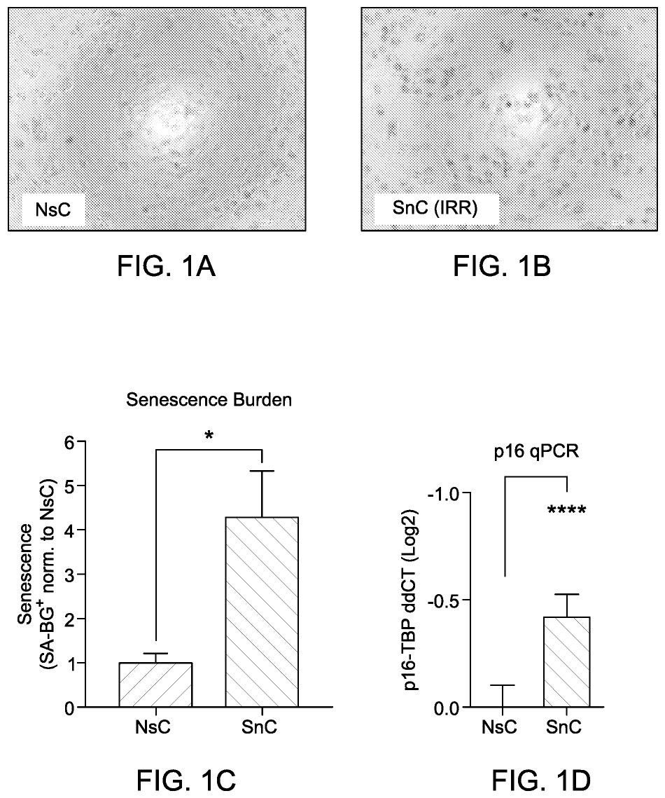

On Day 0, SAEC or HBEC cells were covered with TrypLE trypsin-containing reagent (Thermofisher Scientific®, Waltham, Mass.) and incubated for 8 min until the cells rounded up and began to detach from the plate. Cells were dispersed, counted, and prepared in medium at a concentration of 94,400 cells per mL. This cell suspension was plated in 384-well plates at a volume of 25 μL per well (2360 cells/well). Within 24-hours after cell plating, the 384-well plates were irradiated at 12 Gy to generate senescent cells (SnC). In addition, control 384-well plates were processed in parallel that were not irradiated and served as controls and represent normal, non-senescent cells (NsC). On Day 3, the medium in each well was aspirated and replaced with 25 μL fresh medium. On Day 7, senescence of cells was determined through senescence β-galactosidase staining (Biovision®, Cat. K320-250). To determine induction of the senescence biomarker p16 in irradiated cells, qPCR was performed using Cells-to-CT to measure relative gene expression by real-time RT-PCR and TaqMan detection chemistry (ThermoFisher Scientific®, Cat. A35374) using primers specific for p16 (forward primer: 5′-CTGCCCAACGCACCGAATA-3′ (SEQ ID NO:1); reverse primer: 5′-GCTGCCCATCATCATGACCT-3′ (SEQ ID NO:2); and probe 5′-TTACGGTCGGAGGCCGATCC-3′ (SEQ ID NO.3)) and a housekeeping control gene Tbp (ThermoFisher Scientific®, Cat. 4331182). FIGS. 1A-D demonstrate the ability to induce senescence in primary human epithelial cells by irradiation, where FIG. 1A demonstrates a non-senescent cell, as validated by the detection of senescence β-galactosidase staining (FIGS. 1B and C) and by qPCR detecting p16 (FIG. 1D).

### Example 2: Measuring Bcl/Mcl-1 Inhibition of Candidate Senolytics

The ability of candidate compounds to inhibit a Bcl family activity can be measured on the molecular level by direct binding. This assay uses a homogenous assay technology based on oxygen channeling that is marketed by PerkinElmer Inc., Waltham, Mass.: see Eglin et al., Current Chemical Genomics, 2008, 1, 2-10. The candidate senolytic agent is combined with the target Bcl protein and a peptide representing the corresponding cognate ligand, labeled with biotin. The mixture is then combined with streptavidin bearing luminescent donor beads and luminescent acceptor beads, which proportionally reduces luminescence if the senolytic agent has inhibited the peptide from binding to the Bcl protein.

Bcl-2, Bcl-xL, Bcl-w, and Mcl-1 proteins are available from Sigma-Aldrich Co., St. Louis, Mo. Biotinylated BIM peptide (ligand for Bcl-2, Bcl-xL, and Mcl-1) and BAD peptide (ligand for Bcl-xL) are described in US 2016/0038503 A1. AlphaScreen® Streptavidin donor beads and Anti-6×His AlphaLISA® acceptor beads are available from PerkinElmer

To conduct the assay, a 1:4 dilution series of a senolytic agent is prepared in DMSO, and then diluted 1:100 in assay buffer. In a 96-well PCR plate, the following are combined in order: 10 μL peptide (120 nM BIM or 60 nM BIM), 10 μL of a candidate senolytic agent, and 10 μL Bcl protein (0.8 nM Bcl-2/W or 0.4 nM Bcl-XL or Mcl-1). The assay plate is incubated in the dark at room temperature for 24 h. The next day, donor beads and acceptor beads are combined, and 5 μL is added to each well. After incubating in the dark for 30 minutes, luminescence is measured using a plate reader, and the affinity or degree of inhibition by each senolytic agent is determined.

### Example 3: Measuring Senolytic Activity of Candidate Senolytic Agents in Senescent Fibroblasts

Human fibroblast IMR90 cells can be obtained from the American Type Culture Collection (ATCC®) with the designation CCL-186. The cells are maintained at <75% confluency in DMEM containing FBS and Pen/Strep in an atmosphere of 3% O₂, 10% CO₂, and ˜95% humidity. The cells are divided into groups: irradiated cells (cultured for 14 days after irradiation prior to use) and quiescent cells (cultured at high density for four days prior to use).

On Day 0, the irradiated cells are prepared as follows. IMR90 cells are washed, placed in T175 flasks at a density of 50,000 cells per mL, and irradiated at 10-15 Gy. Following irradiation, the cells are plated at 100 μL in 96-well plates. On Days 1, 3, 6, 10, and 13, the medium in each well is aspirated and replaced with fresh medium.

On Day 10, the quiescent healthy cells are prepared as follows. IMR90 cells are washed, combined with 3 mL of TrypLE trypsin-containing reagent (Thermofisher Scientific, Waltham, Mass.) and cultured for 5 min until the cells have rounded up and begin to detach from the plate. Cells are dispersed, counted, and prepared in medium at a concentration of 50,000 cells per mL. 100 μL of the cells is plated in each well of a 96-well plate. Medium is changed on Day 13.

On Day 14, candidate senolytic agents are combined with the cells as follows. A DMSO dilution series of each test compound is prepared at 200 times the final desired concentration in a 96-well PCR plate. Immediately before use, the DMSO stocks are diluted 1:200 into prewarmed complete medium. Medium is aspirated from the cells in each well, and 100 μL/well of the compound containing medium is added.

Candidate senolytic agents for testing are cultured with the cells for 6 days, replacing the culture medium with fresh medium and the same compound concentration on Day 17. Candidate senolytic agents are cultured with the cells for 3 days. The assay system uses the properties of a thermostable luciferase to enable reaction conditions that generate a stable luminescent signal while simultaneously inhibiting endogenous ATPase released during cell lysis. At the end of the culture period, 100 μL of CellTiter-Glo® reagent (Promega Corp., Madison, Wis.) is added to each of the wells. The cell plates are placed for 30 seconds on an orbital shaker, and luminescence is measured.

### Example 4: Measuring Senolytic Activity of Candidate Senolytic Agents in Senescent HUVEC Cells

Human umbilical vein (HUVEC) cells from a single lot can be expanded in Vascular Cell Basal Media supplemented with the Endothelial Cell Growth Kit™-VEGF from ATCC® to approximately eight population doublings then cryopreserved. Nine days prior to the start of the assay, cells for the senescent population can be thawed and seeded at approximately 27,000/cm₂. All cells are cultured in humidified incubators with 5% CO₂ and 3% O₂ and media changed every 48 hr. Two days after seeding, the cells are irradiated, delivering 12 Gy radiation from an X-ray source. Three days prior to the start of the assay, cells for the non-senescent populations are thawed and seeded as for the senescent population. One day prior to the assay, all cells are trypsinized and seeded into 384-well plates, 5,000/well senescent cells and 10,000/well non-senescent in separate plates in a final volume of 55 μL/well. In each plate, the central 308 wells contained cells and the outer perimeter of wells are filled with 70 μL/well deionized water.

On the day of the assay, candidate senolytic agents can be diluted from 10 mM stocks into media to provide the highest concentration working stock, aliquots of which can then be further diluted in media to provide the remaining two working stocks. To initiate the assay, 5 μL of the working stock can be added to the cell plates. The final test concentrations were 20, 2, and 0.2 μM. In each plate, 100 candidate senolytic agents can be assayed in triplicate at a single concentration along with three wells of a positive control and five no treatment (DMSO) controls. Following senolytic agent addition, the plates are returned to the incubators for three days.

Cell survival can be assessed indirectly by measuring total ATP concentration using CellTiter-Glo™ reagent (Promega®). The resultant luminescence was quantitated with an EnSpire™ plate reader (Perkin Elmer®). The relative cell viability for each concentration of a senolytic agent is calculated as a percentage relative to the no-treatment controls for the same plate.

For follow-up dose responses of candidate senolytic agents, 384-well plates of senescent and non-senescent cells can be prepared as described above. Senolytic agents are prepared as 10-point 1:3 dilution series in DMSO, then diluted to 12× in media. Five microliters of this working stock are then added to the cell plates. After three days of incubation, cell survival relative to DMSO control can be calculated as described above. All measurements can be performed in quadruplicate.

### Example 5: Selectivity of Candidate Senolytic Combinations on Senescent Epithelial Cells

On Day 0, SAEC or HBEC cells were covered with TrypLE trypsin-containing reagent (Thermofisher Scientific®, Waltham, Mass.) and incubated for 8 min until the cells rounded up and began to detach from the plate. Cells were dispersed, counted, and prepared in medium at a concentration of 188,800; 94,400; 47,200; and 23,600 cells per mL. This cell suspension was plated in 384-well plates at a volume of 25 μL per well (4720, 2360, 1180, and 590 cells/well respectively). Within 24-hours after cell plating, the 384-well plates were irradiated at 12 Gy to generate senescent cells (SnC), as described in Example 1. Control 384-well plates were processed in parallel that were not irradiated, representing normal, non-senescent cells (NsC). On Day 3, the medium in each well was aspirated and replaced with 25 μL fresh medium. On Day 7, candidate senolytics were combined with the cells as follows: (1) navitoclax (ABT-263) alone; and (2) navitoclax (ABT-263)+AMG-176 at two different doses—2.5 μM and 0.5 μM. A 13-pt dilution series of each senolytic (in DMSO) was prepared at 1000 times the final desired concentration in a 384-well plate. Immediately before use, the DMSO stocks were diluted 1:1000 into prewarmed complete medium. Medium was aspirated from the cells in each well, and 25 μL/well of the senolytic, in this case navitoclax, containing medium was added. Next, a second senolytic, in this case AMG-176, an Mcl-1 inhibitor, was added using a Tecan D300e Digital Dispenser at a fixed concentration.

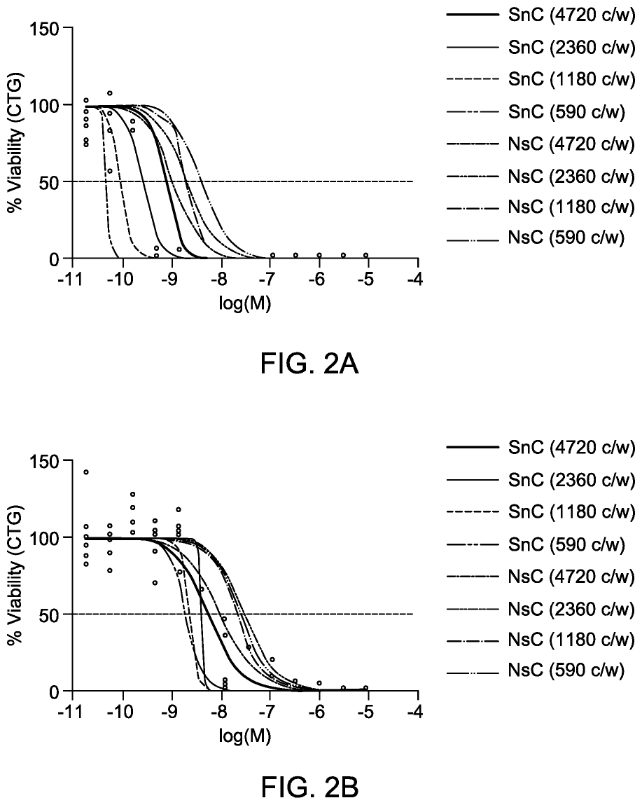

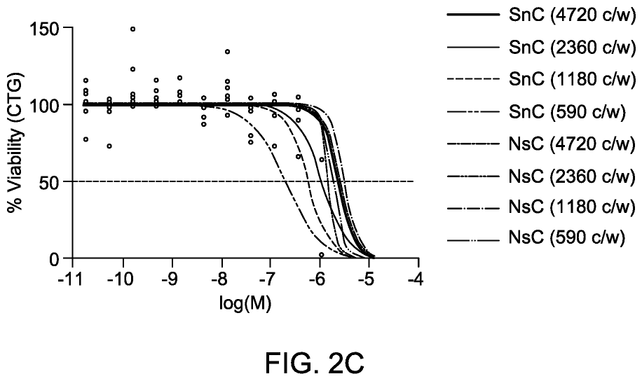

The candidate senolytics were cultured with the senescent cells for 3 days. The assay system used the properties of a thermostable luciferase to enable reaction conditions that generate a stable luminescent signal while simultaneously inhibiting endogenous ATPase released during cell lysis. On Day 10, the end of the culture period, the plates were removed from the incubator and allowed to equilibrate at room temperature for 15 minutes then 25 μL of CellTiter-Glo® reagent (Promega® Corp., Madison, Wis.) was added to each of the wells. The cell plates were placed for 30 seconds on an orbital shaker and then allowed to stand at room temperature for 30 minutes before measuring luminescence. The luminescence readings were normalized to determine % cell survival/growth and plotted against candidate senolytic concentrations, and potencies expressed as EC50 values were determined by non-linear curve fitting in Graphpad Prism. FIG. 2A-C demonstrates the results.

The concentration-response curve for the senolytic combination of navitoclax and AMG-176 (FIG. 2A, B) demonstrates sensitivity of senescent lung epithelial cell survival (SnC) to incubation with a senolytic, whereas this senolytic combination shows limited senolysis in non-senescent cells (NsC). Including a senolytic combination increases the senolytic potency while retaining selectivity in senescent cells. These data show that senolytic combinations are capable of selectively eliminating senescent lung airway cells in culture.

### Example 6: Bcl-xL and Mcl-1 Target Engagement Pharmacodynamics

A target engagement assay was developed to determine Bcl-xL and Mcl-1's respective interaction with BIM both in vivo in mice, or cells in culture, in order to predict senolysis. The respective binding pockets of Bcl-xL and Mcl-1 proteins under normal conditions sequestrate pro-apoptotic proteins, such as BIM. Co-immunoprecipitation (co-IP) was employed to study protein-protein interactions in order to determine if candidate senolytics can effectively displace Bcl-xL or Mcl-1 from BIM protein.

Lysates for co-IP experiments were prepared from: (a) lungs from mice that had been dosed for 8 hours by oral aspiration (OA) with either a candidate senolytic Bcl (Compound 1) or Mcl-1 inhibitor (S-63845), or a control vehicle treatment; or (b) mouse bronchiotrachael epithelial (MBE) cells, dosed for 4 hours with the same Bcl or Mcl-1 inhibitor, using a non-denaturing lysis buffer: PBS pH 7.4, 2% CHAPS, supplemented with a protease inhibitor cocktail (Roche®)). For every 0.25 gram of tissue 1 mL of lysis buffer was added to the sample, and next tissue was homogenized using a Precellys 24 (Bertin Technologies®; 3 cycles of 20 s and 20 s pause). Homogenates were cleared by centrifugation at 13000 g at 4° C. for 10 min, and the supernatant lysate was transferred to new tubes, aliquoted, flash-frozen using liquid nitrogen, and stored at −80° C.

Immunoprecipitations were performed using rabbit monoclonal anti-BIM (C34C5) antibodies (Cell Signaling Technology®) bound by Protein A-coated magnetic beads (Life Technologies®). 100 ul lysate was incubated with 10 μL of magnetic beads (pre-coated with 2 μg of antibody) for 1 hour at 4° C. in a Thermal Mixer at 1300 rpm (VWR®). After immunocapture, samples were washed three times with cold lysis buffer and eluted from the beads using non-reducing NuPAGE LDS Sample Buffer® (Life Technologies®). The eluted BIM co-immunoprecipitates were separated on a NuPAGE 4-12% Bis-Tris Gel using MES running buffer (Life Technologies®), and transferred on a nitrocellulose membrane using the iBlot Gel Transfer System® (Life Technologies®) according to the manufacturer's instructions.

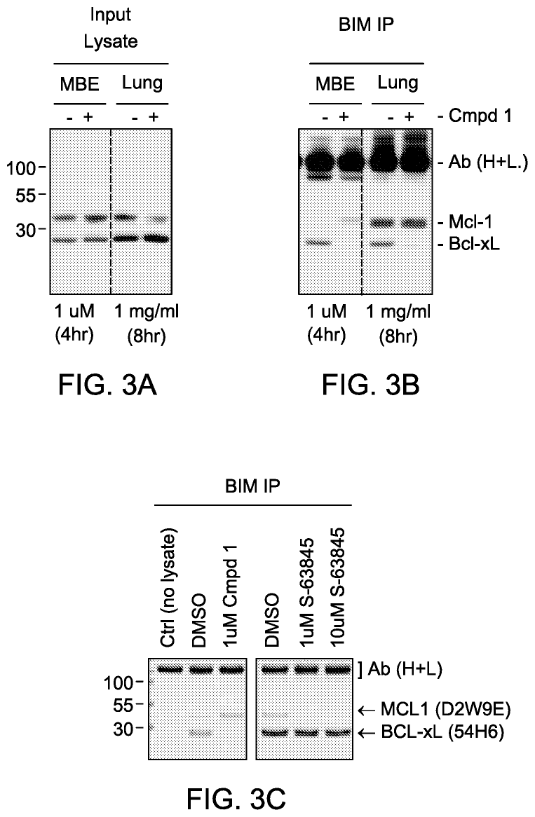

For western blot detection of Bcl-xL and Mcl-1 proteins, in both lysates and co-immunoprecipitates, rabbit monoclonal antibodies were used simultaneously at 1:1000 (Cell Signaling Technology®; clones 54H6 and D2W9E, respectively). Anti-rabbit IgG, HRP-linked antibody (Cell Signaling Technology®) was used as a secondary antibody at 1:10000. Chemiluminescence was performed using the SuperSignal Chemiluminescence Kit® (Pierce®) according to the manufacturer's instructions and images were captured using an Azure Western Blot Imaging System®. Results are shown in FIGS. 3A-C, which demonstrate that in both OA-dosed mouse lungs and MBE cells (FIG. 3A-C), when BIM-Bcl-xL interactions were blocked by the aryl sulfonamide Bcl-2/Bcl-xL inhibitor Compound 1 (FIG. 3B), a compensatory Mcl-1 binding to BIM was observed (FIG. 3B). In FIG. 3C, Bcl-xL appeared to compensate for BIM binding when Mcl-1 was inhibited by the Mcl-1 inhibitor S-63845 at two concentrations 1 uM and 10 uM. In sum, this suggests that both Bcl-xL and Mcl-1 need to be displaced for efficient senolysis.

### Example 7: Synergistic Efficacy of Senolytic Combinations on Senescent Epithelial Cells

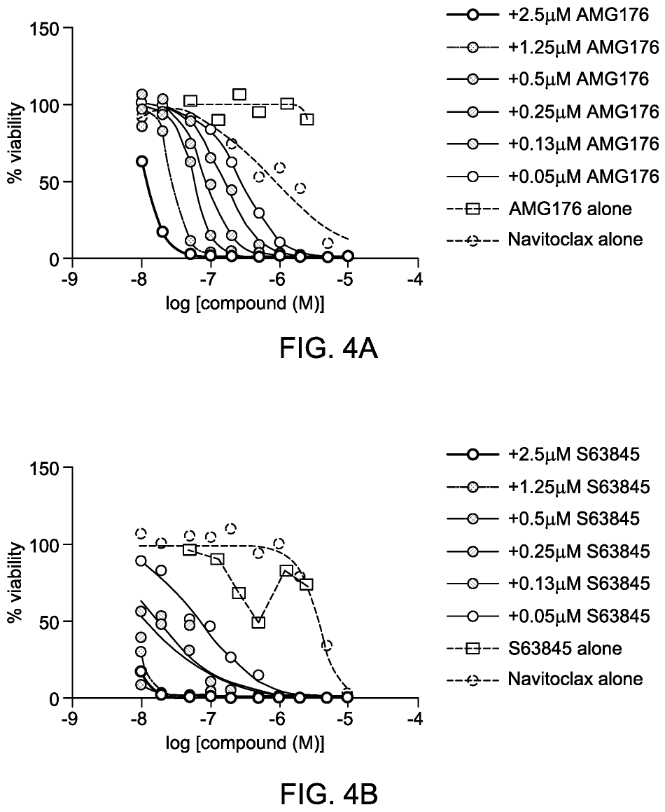

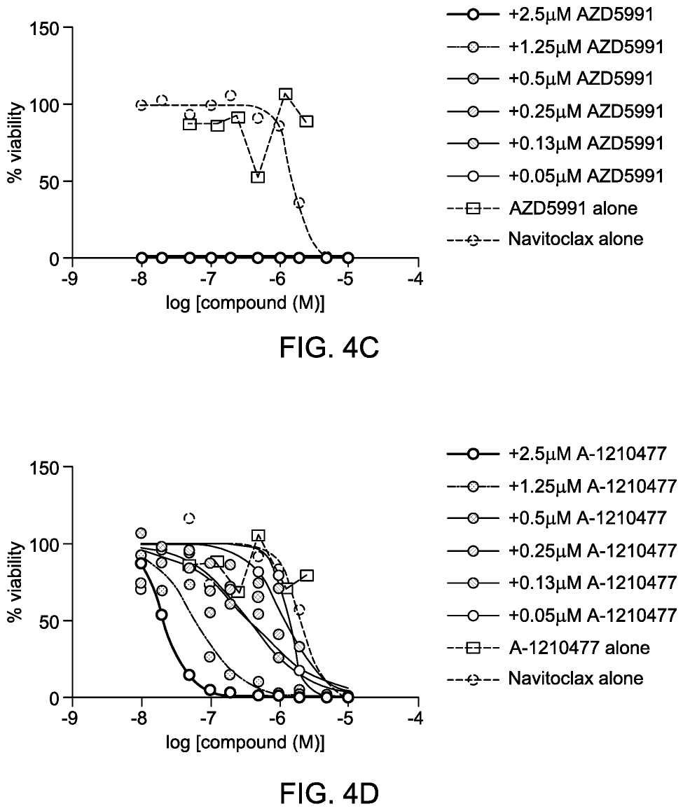

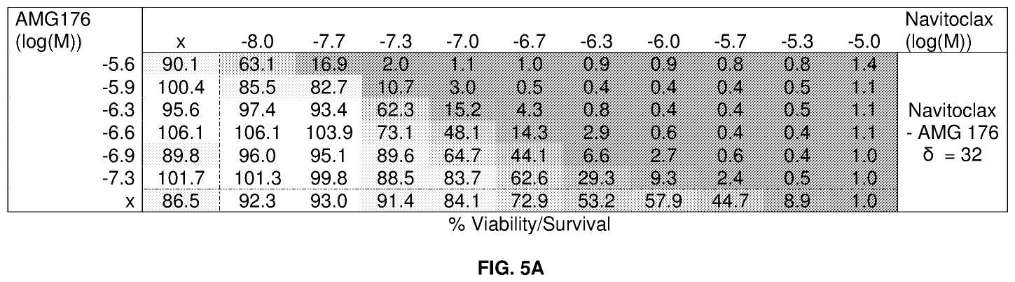

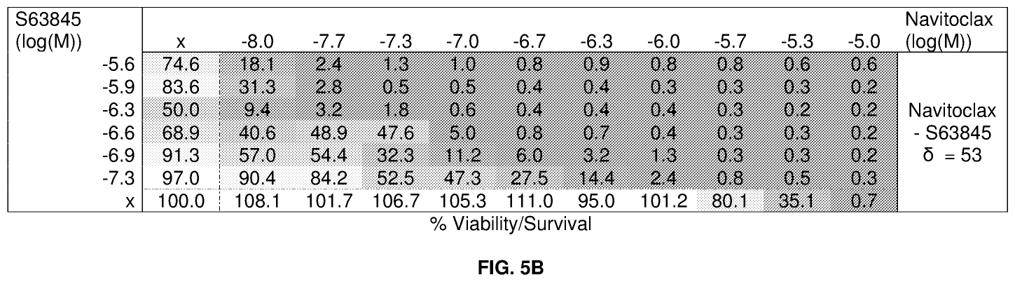

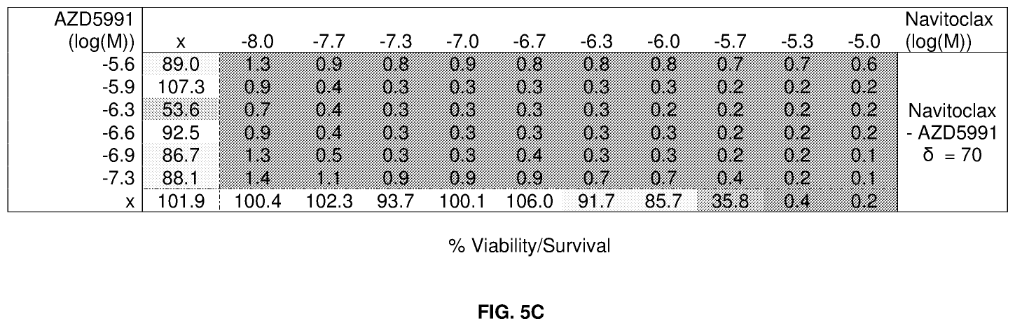

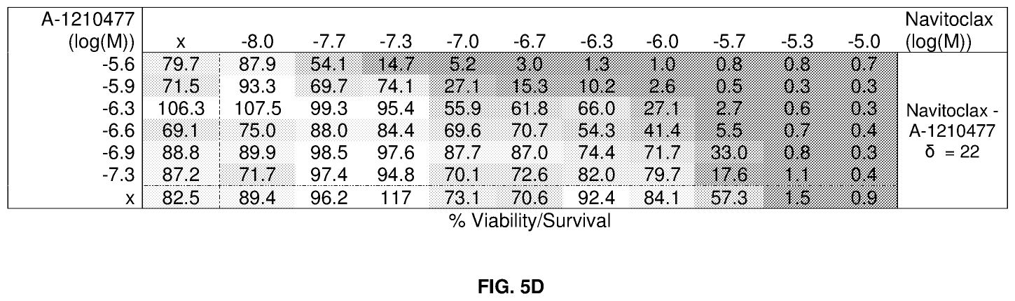

Results from Example 6 suggested that both Bcl-xL and Mcl-1 needed to be displaced from binding to BIM to observe senolysis. Thus, particular Bcl inhibitor and Mcl-1 inhibitor combinations were tested for their senolytic potential by performing dose-response matrices on senescent cells. Primary human SAECs were made senescent as described in Example 1. Fresh media was added on Day 7 and candidate senolytic combinations were added in a dose-response matrix on a 384-well plate in a 11×7 well format. On the x-axis, Bcl inhibitors were tested at the following final concentrations: 0, 0.010, 0.022, 0.046, 0.1, 0.22, 0.46, 1.00, 2.15, 4.64, 10 μM (left to right), whereas on the y-axis an Mcl-1 inhibitor was added at 2.50, 1.16, 0.54, 0.25, 0.12, 0.05 μM (top to bottom). Candidate senolytics in dimethyl sulfoxide (DMSO) were added using a Tecan® D300e Digital Dispenser (Tecan Life Sciences®). Each plate also included a similar matrix in which the candidate senolytic was substituted with DMSO to serve as a viability normalization control. The candidate senolytics were cultured with the senescent SAECs for 3 days. On Day 10, the end of the assay period, the plates were removed from the incubator and allowed to equilibrate at room temperature for 15 minutes. Then, 25 μL of CellTiter-Glo® reagent (Promega® Corp., Madison, Wis.) was added to each of the wells. The assay system used the properties of a thermostable luciferase to enable reaction conditions that generate a stable luminescent signal while simultaneously inhibiting endogenous ATPase released during cell lysis. The cell plates were placed for 30 seconds on an orbital shaker and then allowed to stand at room temperature for 30 minutes before measuring luminescence. The luminescence readings were normalized to the DMSO controls to determine % cell survival/growth and plotted against candidate senolytic concentrations. Two different results were calculated from these dose-response matrixes: (a) dose-responses for cell viability expressed as an EC50 (FIG. 4A-D); and (b) the degree of synergistic senolysis, or synergistic coefficient, expressed as a δ value (FIG. 5A-D), as described in detail below.

Synergistic senolysis was calculated using the Zero Interaction Potency Delta (δ) methodology as described in Yadav et al., Comput Struct Biotechnol J. 2015; 13: 504-513, which is incorporated by reference. Basically, “delta” (δ) indicates the degree of synergy achieved and was calculated using equation (19) as described in Yadav et al 2015 and herein. For example, a δ=0.2 corresponds to 20% of response beyond expectation). Thus, the larger the δ value, the stronger the synergistic senolysis. The delta scoring requires the parameters for the dose-response curves both in monotherapy and in combination and at least three dose-response data points. A delta score can be calculated for each senolytic dose combination in the matrix, which allows for a surface plot of delta scores. Such a surface plot enables one to characterize drug interaction effects over the full dose matrix, which is more informative than what a single summary score can provide.

The results of the various senolytic combinations by (a) EC50 (FIG. 4A-D); and (b) by the synergistic coefficient “delta” (δ) (FIG. 5A-D) are as follows. FIG. 4A-D and FIG. 5A-D all show synergistic senolysis with the Bcl-2/Bcl-xL inhibitor navitoclax in combination with four different Mcl-1 inhibitors tested: AMG-176 (FIGS. 4A and 5A), S-63845 (FIGS. 4B and 5B), AZD-5991 (FIGS. 4C and 5C), and A-1210477 (FIGS. 4D and 5D).

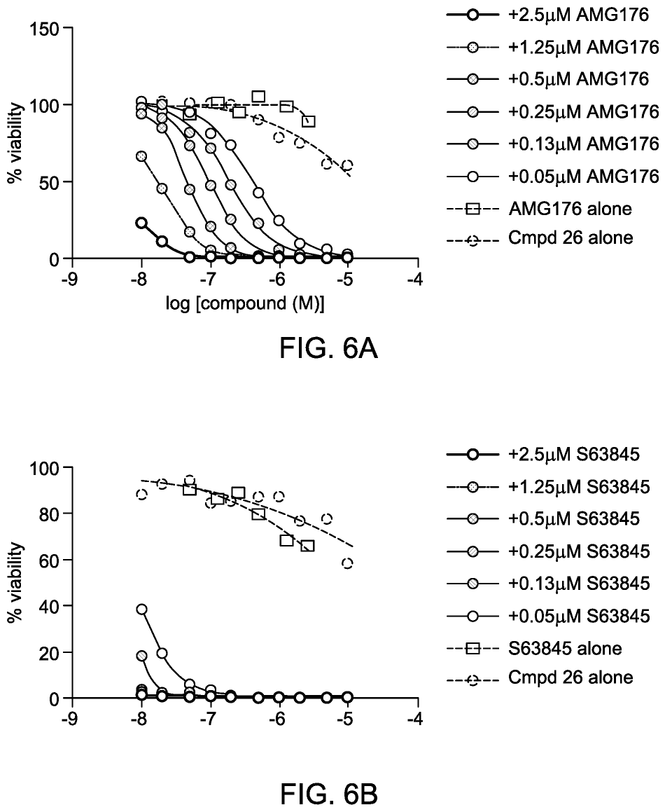

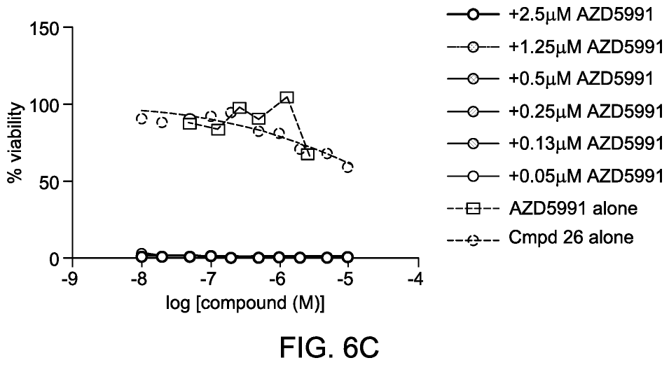

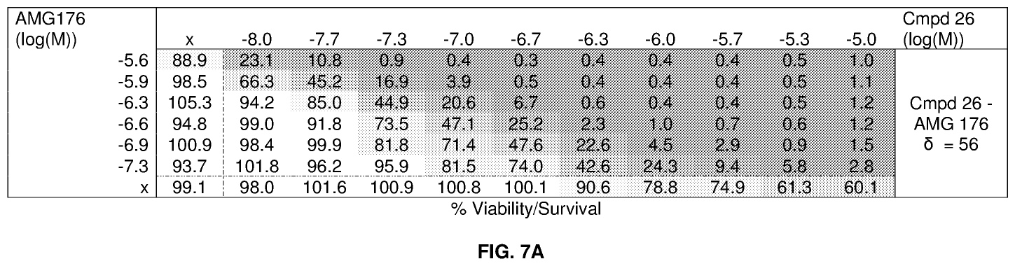

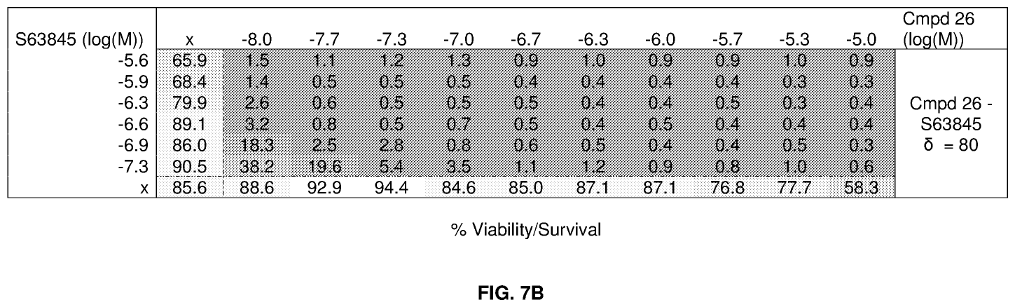

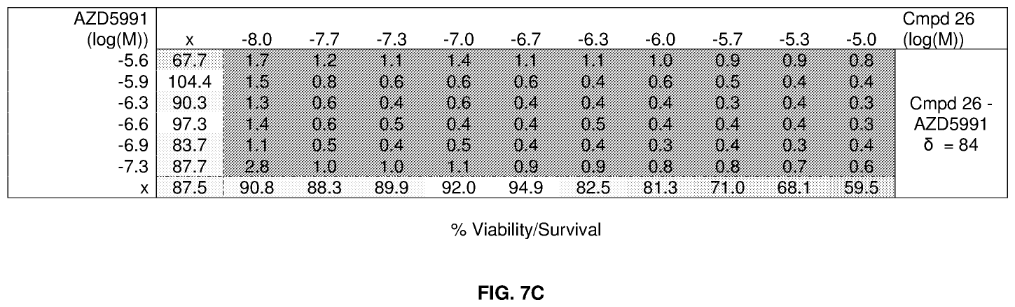

FIG. 6A-C and FIG. 7A-C all show synergistic senolysis with an aryl sulfonamide Bch 2/Bcl-xL inhibitor Compound 26 in combination with three different Mcl-1 inhibitors: AMG-176 (FIGS. 6A and 7A), S-63845 (FIGS. 6B and 7B), and AZD-5991 (FIGS. 6C and 7C).

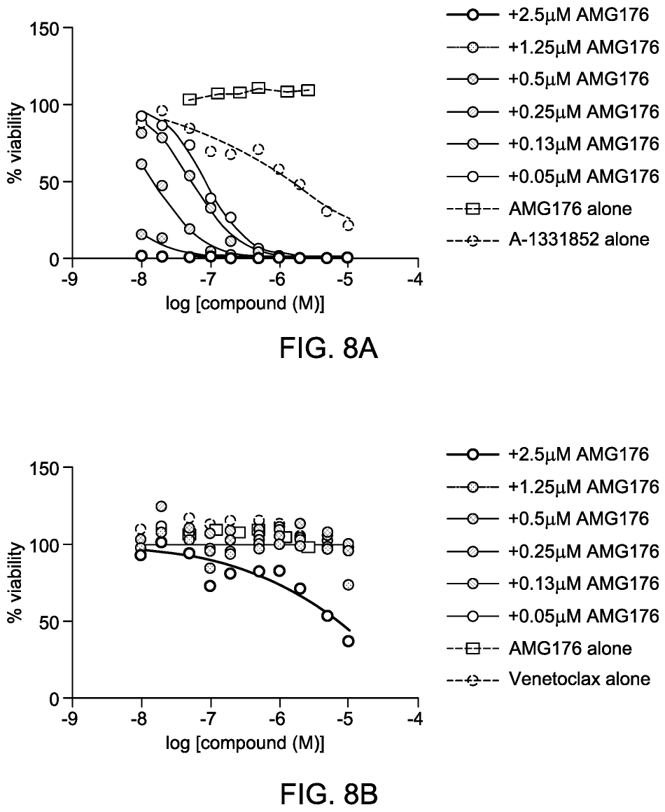

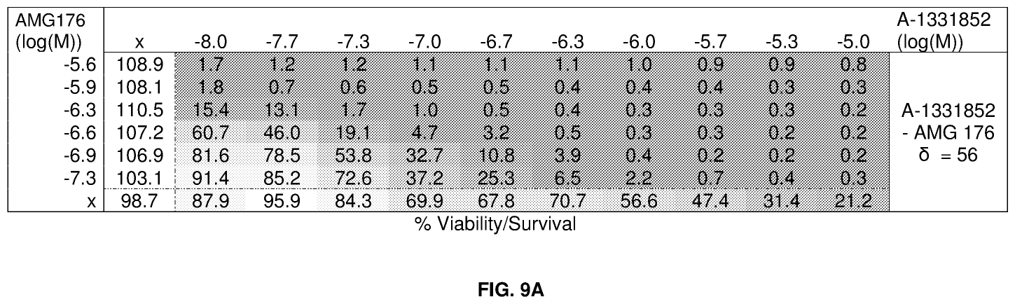

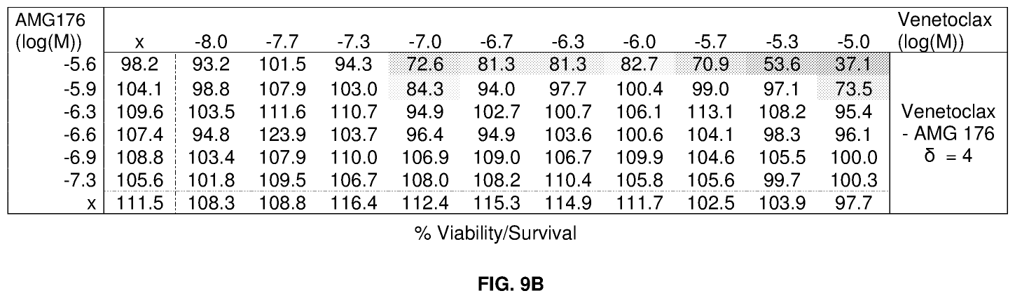

FIGS. 8A and 9A both show synergistic senolysis with a Bcl-xL selective inhibitor A-1331852 in combination with the Mcl-1 inhibitor AMG-176. However, combining Venetoclax, a known Bcl-2-selective inhibitor, and the Mcl-1 inhibitor AMG-176 demonstrated poor senolysis and no synergy (FIGS. 8B and 9B), suggesting that senolytic synergy may require Bcl-xL inhibition and that Bcl-2 inhibition alone is not sufficient for effective senolysis.

### Example 8: Senolysis Assessment in the Idiopathic Pulmonary Fibrosis Pharmacodynamic Model

Senescence was induced in the lungs of mice using bleomycin. Mouse lung cells were processed to enrich for lung epithelial cells. Epithelial cells are thought to be major contributors to the inflammation and fibrosis associated with many human interstitial lung diseases. Post-bleomycin administration, Bcl and Mcl-1 inhibitors were administered in combination to determine their senolytic potency towards eliminating senescent epithelial cells. Senescence can be measured using qPCR, flow cytometry and immunohistochemistry (IHC). Induction of apoptosis in senescent cells was measured by caspases as described herein.

Bleomycin senescence induction: Senescence was induced in mouse lungs using oral aspiration (OA) delivery of bleomycin. Bleomycin is a DNA damaging agent that induces senescence, inflammation and fibrosis in the lungs. Briefly, between 10 to 20 4-8-week-old male c57Bl/6 mice per experimental group were OA dosed with either ˜2.2 Units/Kg of bleomycin (formulated in PBS) or PBS vehicle. Weight and health were monitored daily. Mice that lost more than 20% of their starting weight prior to treatment were excluded from the study.

Administration of candidate Bcl and Mcl-1 inhibitor combinations: Bcl and Mcl-1 inhibitors were administered 14 days post-bleomycin induction via OA delivery. In the experiment described herein, the Bcl inhibitor Compound 1 was administered OA (50 μl; 1 mg/ml) and then 1 hour later the Mcl-1 inhibitor AZD-5991, was administered OA (50 μl; 0.3 mg/ml, 0.5 mg/ml, or 1 mg/ml). The appropriate vehicle control was also included.

### Lung Processing—Single Cell Preparation

At various time points after dosing, mice Lung Processing—Single Cell Preparation: Five hours after Bcl and Mcl-1 inhibitor combination administration, mice were anesthetized, and the lungs were perfused with PBS and isolated for processing. The left lung was fixed in optimal cutting temperature (OCT) compound for IHC analysis while the right lung was processed to make a single cell suspension. Briefly, right lungs were minced with scissors and then incubated in a collagenase cocktail (30 mg collagenase type 2 (Worthington Biochemical Corp. Cat LS-004177), 30 mg hyaluronidase (Sigma Cat #H3506), 30 mg dispase (Sigma Cat D-4693), 5000 U DNAse (Sigma Cat #4536282001) 2.5% FBS (VWR Cat #97068-085) in DMEM F-12 (ATCC Cat #30-2006) for lhr at 37° C. This suspension was then incubated for 5 min at room temperature with Biovision® RBC lysis buffer (Cat #5830-100). After lysis the suspension was centrifuged for 5 min at 350 g. The pelleted cells are then resuspended in DMEM with 5% FBS and filtered through a 70 μm filter. Cells were counted after filtration in preparation for CD45+ cell depletion, discussed below.

### CD45/CD31 Depletion

CD45/CD31 Depletion: To remove CD45(+)/CD31(+) immune cells, the single cell lung preps were incubated with 10% CD45(+)/CD31(+) microbeads (Stemcell Technology Cat #60030BT/BioLegend Cat #102504) for 15 min at 4° C. in a microtiter plate. After incubation the cells were centrifuged at 350 g for 5 min. The CD45(+) cells were removed by magnetic separation after several washes with buffer. The desired CD45(−) were counted and then used for epithelial cell adhesion molecule (EpCAM) enrichment (described below).

### EpCAM Enrichment

Epithelial cells exhibit increased expression of EpCAM, an important transmembrane glycoprotein involved in cell adhesion. The CD45(−) cell suspension was incubated with microbeads conjugated to an anti-CD326 antibody (eBioscience/Invitrogen Cat #12-5791-83) for 15 min at 4° C. Magnetic separation was used to remove EpCAM (+) cells from the cell population. Epcam(−) cells were removed after washing with MACS buffer. The EpCAM (+) cells were then released using nanoparticles. The resulting cells were counted and should be CD45(−)/CD31(−) and EpCAM (+). Aliquots of these cells were then used to confirm cell enrichment via flow cytometry and to quantify senescence via qPCR.

Senescence qPCR

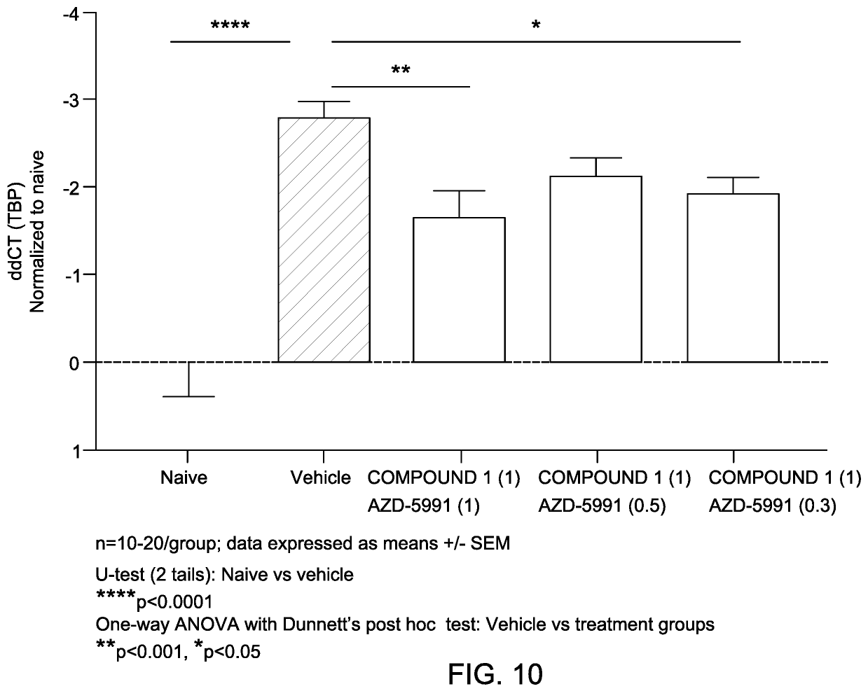

RNA was isolated from CD45(−)/EpCAM(+) using standard trizol protocols (Trizol Cat #15596026). A multiplexed qPCR reaction was run using cDNA encoded from 500 ng isolated RNA using the Superscript 4 kit (Invitrogen Cat #18091050). The PCR reaction utilizes Taqman™ expression assays which contain primers specific for p16 (IDT; Sequence Information: Fwd: 5′-AAC TCT TTC GGT CGT ACC CC-3′; Rvs: 5′-TCC TCG CAG TTC GAA TCT G-3′; Probe: 5′-/56-FAM/AGG TGA TGA/ZEN/TGA TGG GCA ACG TTC AC/3IABkFQ/-3′), TATA box binding protein (TBP) and actin. p16 is quantified using the ΔΔ Ct method using actin and TBP as reference genes to normalize the expression levels. FIG. 10 shows the effect of Compound 1 in combination with AZD-5991 in bleomycin-induced p16 expression in mouse lung epithelial cells. Data was expressed as means+/−SEM, U-test (2 tails): naïve versus vehicle. ****p<0.0001. One-way ANOVA with Dunnett's post hoc test: vehicle versus treatment groups **p<0.001, *P<0.05.

### Caspase Assay

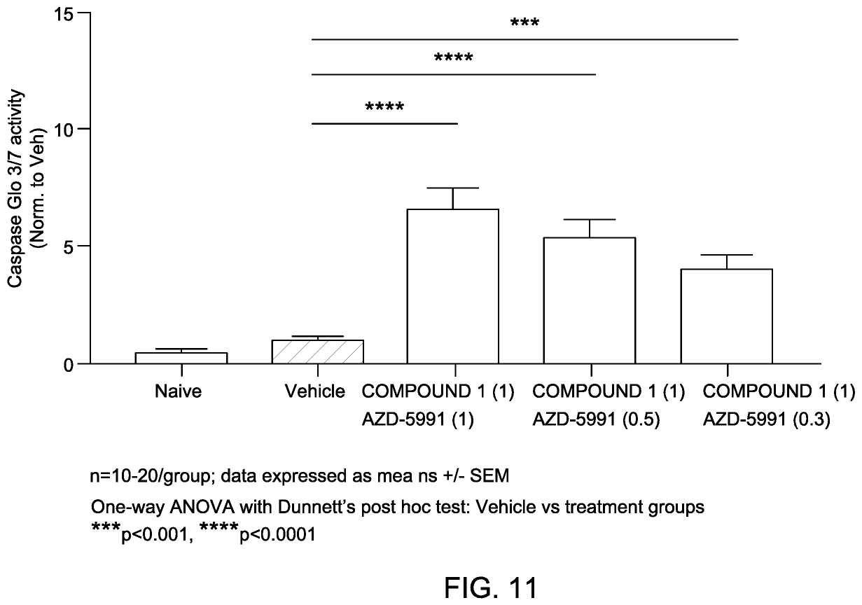

To measure apoptosis, whole lungs were homogenized using Precellys bead homogenization. One microliter of the whole lung lysate was added to the Caspase-Glo® 3/7 Assay System (Promega® Cat #G8090). The 1 μl lysate was also diluted 10-fold to ensure linearity. The mixture was incubated for 30 min at room temperature and luminosity was measured for each sample using a Perkin Elmer Enspire®. FIG. 11 shows the effect of Compound 1 in combination with AZD-5991 on caspase 3/7 activity in bleomycin-induced mouse lung epithelial cells. Data was expressed as means+/−SEM. One-way ANOVA with Dunnett's post hoc test: vehicle versus treatment groups ***p<0.001, ****p<0.0001.

### Example 9: Efficacy of Senolytic Agents in a Pulmonary Disease Model

This example illustrates the testing of candidate senolytic combinations in a mouse model for treatment of lung disease: specifically, as a model for chronic obstructive pulmonary disease (COPD), in which mice are exposed to cigarette smoke. The effect of candidate senolytic agents in combination on the mice exposed to smoke is assessed by senescent cell clearance, lung function, and histopathology.

The mice to be used in this study include the 3MR strain, described in US 2017/0027139 A1 and in Demaria et al., Dev Cell. 2014 Dec. 22; 31(6): 722-733. The 3MR mouse has a transgene encoding thymidine kinase that converts the prodrug gancyclovir (GCV) to a compound that is lethal to cells. The enzyme in the transgene is placed under control of the p16 promoter, which causes it to be specifically expressed in senescent cells. Treatment of the mice with GCV eliminates senescent cells.

Other mice to be used in this study include the INK-ATTAC strain, described in US 2015/0296755 A1 and in Baker et al., Nature 2011 Nov. 2; 479(7372):232-236. The INK-ATTAC mouse has a transgene encoding switchable caspase 8 under control of the p16 promoter. The caspase 8 can be activated by treating the mice with the switch compound AP20187, whereupon the caspase 8 directly induces apoptosis in senescent cells, eliminating them from the mouse.

To conduct the experiment, six-week-old 3MR or INK-ATTAC mice can be chronically exposed to cigarette smoke generated from a Teague TE-10 system, an automatically-controlled cigarette smoking machine that produces a combination of side-stream and mainstream cigarette smoke in a chamber, which is transported to a collecting and mixing chamber where varying amounts of air is mixed with the smoke mixture. The COPD protocol was adapted from the COPD core facility at Johns Hopkins University (Rangasamy et al., 2004, J. Clin. Invest. 114:1248-1259; Yao et al., 2012, J. Clin. Invest. 122:2032-2045).

Mice can receive a total of 6 hours of cigarette smoke exposure per day, 5 days a week for 6 months. Each lighted cigarette (3R4F research cigarettes containing 10.9 mg of total particulate matter (TPM), 9.4 mg of tar, and 0.726 mg of nicotine, and 11.9 mg carbon monoxide per cigarette [University of Kentucky, Lexington, Ky.]) was puffed for 2 seconds and once every minute for a total of 8 puffs, with the flow rate of 1.05 L/min, to provide a standard puff of 35 cm³. The smoke machine can be adjusted to produce a mixture of side stream smoke (89%) and mainstream smoke (11%) by smoldering 2 cigarettes at one time. The smoke chamber atmosphere was monitored for total suspended particulates (80-120 mg/m³) and carbon monoxide (350 ppm).

Beginning at day 7, INK-ATTAC and 3MR mice are treated with AP20187 (3× per week) or gancyclovir (5 consecutive days of treatment followed by 16 days off drug, repeated until the end of the experiment), respectively. An equal number of mice received a corresponding vehicle as control. The remaining mice are evenly split and can be placed into three different treatment groups. One group can receive a test Bcl inhibitor in combination with a test Mcl-1 inhibitor at doses suitable for the necessary pK and PD. One group can receive the test Bcl inhibitor alone or the test Mcl-1 inhibitor alone, and the last group can receive only the vehicle as a control, following the same treatment regimen as the test inhibitors. Additional mice that did not receive exposure to cigarette smoke were used as controls for the experiment.

After two months of cigarette smoke (CS) exposure, lung function can be assessed by monitoring oxygen saturation using the MouseSTAT PhysioSuite™ pulse oximeter (Kent Scientific). Animals are anesthetized with isoflurane (1.5%) and the toe clip is applied. Mice are monitored for 30 seconds and the average peripheral capillary oxygen saturation (SpO₂) measurement over this duration can be calculated.

### Example 10: Efficacy of Senolytic Agents in an In Vivo Osteoarthritis Model

Candidate senolytics in combination may be tested in a mouse model for treatment of osteoarthritis as follows. C57BL/6J mice can undergo surgery to cut the anterior cruciate ligament of one rear limb to induce osteoarthritis in the joint of that limb. During week 3 and week 4 post-surgery, the mice can be treated with candidate senolytics in combination per operated knee by intra-articular injection, q.o.d. for 2 weeks. At the end of 4 weeks post-surgery, joints of the mice may be monitored for the presence of senescent cells, assessed for function, monitored for markers of inflammation, and histological assessment.

Two control groups of mice can be included in the studies performed: one group comprising C57BL/6J mice that undergo a sham surgery (i.e., surgical procedures followed except for cutting the ACL) and intra-articular injections of vehicle parallel to the senolytic treated group; and one group comprising C57BL/6J mice that undergo an ACL surgery and received intra-articular injections of vehicle parallel to the senolytic-treated group. RNA from the operated joints of mice from the senolytic-treated mice can be analyzed for expression of SASP factors, such as, for example, IL-6, and senescence markers, such as, for example, p16. qRT-PCR can be performed to detect mRNA levels.

Function of the limbs can be assessed 4 weeks post-surgery by a weight bearing test to determine which leg the treated mice favored. The mice can be allowed to acclimate to the chamber on at least three occasions prior to taking measurements. Mice may be maneuvered inside the chamber to stand with one hind paw on each scale. The weight that is placed on each hind limb can be measured over a three second period. At least three separate measurements can be made for each animal at each time point. The results are then expressed as the percentage of the weight placed on the operated limb versus the contralateral unoperated limb.

### Example 11: Efficacy of Senolytic Agents in a Bleomycin-Induced Glaucoma Model

This example illustrates the testing of a Bcl inhibitor in combination with an Mcl-1 inhibitor in a mouse model for treatment of an eye disease, specifically primary open angle glaucoma (POAG).

Male C57Bl6/J mice aged 8-10 weeks can be sedated in isofluorane chamber for 3 min then placed on operating table in a nose-cone to maintain constant isofluorane anesthesia. One drop of 2.5% phenylephrine-tropicamide is deposited on the eye for dilation. Measurement of baseline intra-ocular pressure (TOP) can be taken on both eyes using Tonolab™ prior to surgery. The IOP value is reported as an average of six measurements. To induce glaucoma-like phenotype, two μL of bleomycin (0.25 U/kg) or PBS (control) can be intra-camerally injected in the right eye.

IOP measurements can be performed at Day 7 (before treatment), 14, and 21 days after injury. Treatment can be performed 7 days after bleomycin injury. Mice can be sedated in an isofluorane chamber for 3 min then placed on operating table in a nose-cone to maintain constant isofluorane anesthesia. One drop of 2.5% phenylephrine-tropicamide can be deposited on the eye for dilation. Microliter volumes at suitable concentrations of the candidate senolytic agents in combination or vehicle only can be intra-camerally injected into one eye.

Eye samples can be collected 14 and 21 days after bleomycin injury. Trabecular meshwork can be collected and fast frozen in liquid nitrogen. Storage of the samples can be at −80° C. until RNA extraction. RNA extraction can be performed using chloroform extraction followed by use of the Direct-Zol Microprep™ RNA extraction kit (VWR®). Five hundred nanograms of RNA can be used to prepare cDNA using the High Capacity Reverse Transcriptase™ kit (ThermoFisher®). One tenth of the cDNA can be used for level of RNA expression measurements using the PerfeCTa qPCR ToughMix Low Rox™ and Taqman™ primer/probe (QuantaBio™)

### Example 12: Efficacy of Senolytic Agents in an Animal Model of Diabetes Induced Retinopathy

The streptozotocin (STZ) rodent model (Feit-Leichman et al, IOVS 46:4281-87, 2005) recapitulates features of diabetic retinopathy and diabetic macular edema through the induction of hyperglycemia via the direct cytotoxic action of STZ on pancreatic beta cells. Hyperglycemia occurs within days following STZ administration and phenotypic aspects of diabetic retinopathy occur within weeks, with vascular leakage and reduced visual acuity and contrast sensitivity demonstrated in these rodents. This model has thus been widely used for the evaluation of therapeutic agents in diabetic eye disease.

C57BL/6J mice of 6- to 7-weeks are weighed and their baseline glycemia are measured (Accu-Chek®, Roche). Mice can be injected intraperitoneally with STZ (Sigma-Alderich®, St. Lois, Mo.) for 5 consecutive days at 55 mg/Kg. Age-matched controls can be injected with buffer only. Glycemia can be measured again a week after the last STZ injection and mice are considered diabetic if their non-fasted glycemia is higher than 17 mM (300 mg/dL). STZ treated diabetic C57BL/6J mice can be intravitreally injected with microliter volumes of candidate senolytic agents at 8 and 9 weeks after STZ administration. Retinal Evans blue permeation assay can be performed at 10 weeks after STZ treatment.

### Example 13: Effect of Senolytic Agents in Animal Models of Atherosclerosis

Candidate senolytics in combination may be tested in a mouse model for treatment of atherosclerosis utilizing the LDLR^{-/-} mice (The Jackson Laboratory), that have a Ldlr^{tm1Her} mutation resulting in an elevated serum cholesterol level, and can be induced to have very high levels of serum cholesterol when fed a high fat diet, as follows.

Two groups of LDLR^{-/-} mice (10 weeks) can be fed a commercially available murine high fat diet (HFD) of Harlan Teklad TD.88137, having 42% calories from fat, beginning at Week 0 and throughout the study. Two groups of LDLR^{-/-} mice (10 weeks) can be fed normal chow (−HFD). From weeks 0-2, one group of HFD mice and −HFD mice are treated with candidate senolytic agents in combination. One treatment cycle is 14 days treatment, 14 days off. Vehicle is administered to one group of HFD mice and one group of −HFD mice. At week 4 (time point 1), one group of mice are sacrificed and to assess presence of senescent cells in the plaques. For the some of the remaining mice, candidate senolytic agent treatment and vehicle administration is repeated from weeks 4-6. At week 8 (timepoint 2), the mice can be sacrificed to assess the presence of senescent cells in the plaques. The remaining mice are treated with candidate senolytic agents or vehicle from weeks 8-10. At week 12 (timepoint 3), the mice are sacrificed and to assess the level of plaque and the number of senescent cells in the plaques.

Plasma lipid levels can be measured in LDLR^{-/-} mice fed a HFD and treated with candidate senolytic agents or vehicle at time point 1 as compared with mice fed a −HFD. Plasma can be collected mid-afternoon and analyzed for circulating lipids and lipoproteins. Clearance of senescent cells with candidate senolytic agents in LDLR^{-/-} mice fed a HFD can be assessed and the expression levels of several SASP factors and senescent cell markers, MMP3, MMP13, PAH, p21, IGFBP2, IL-1A, and IL-1B after 1 treatment cycle can also be measured by RT-PCR analysis. At the end of time point 2, aortic arches can be dissected for RT-PCR analysis of SASP factors and senescent cell markers.

At the end of time point 3, LDLR^{-/-} mice fed a HFD and treated with candidate senolytic agents or vehicle can be sacrificed, and aortas dissected and stained with Sudan IV to detect the presence of lipid. Body composition of the mice can be analyzed by MRI, and circulating blood cells can be counted by an automated hematology system, Hemavet™ (Drew Scientific Group).

The several hypotheses presented in this disclosure provide a premise by way of which the reader may understand the invention. This premise is provided for the enrichment and appreciation of the reader. Practice of the invention does not require detailed understanding or application of the hypothesis. Except where stated otherwise, features of the hypothesis presented in this disclosure do not limit application or practice of the claimed invention. For example, except where the elimination of senescent cells is explicitly required, the senolytic combinations of this invention may be used for treating the conditions described regardless of their effect on senescent cells. Although many of the senescence-related conditions referred to in this disclosure occur predominantly in older patients, the invention may be practiced on patients of any age having the condition indicated, unless otherwise explicitly indicated or required.

While the invention has been described with reference to the specific examples and illustrations, changes can be made and equivalents can be substituted to adapt to a particular context or intended use as a matter of routine development and optimization and within the purview of one of ordinary skill in the art, thereby achieving benefits of the invention without departing from the scope of what is claimed.

What is claimed is:

1. A method for treating a senescence-associated disease or disorder comprising administering to a subject in need thereof therapeutically-effective amounts of a Bcl inhibitor and an Mcl-1 inhibitor.

2. The method of claim 1, wherein said Bcl inhibitor and Mcl-1 inhibitor selectively kill senescent cells.

3. A method for selectively killing a senescent cell, comprising contacting the cell with an effective amount of a senolytic combination, wherein the senolytic combination is a means for inhibiting Bcl and a means for inhibiting Mcl-1.

4. A method of enhancing the senolytic activity of a Bcl inhibitor and/or the therapeutic efficacy of the Bcl inhibitor for treating a senescence associated disease or disorder, wherein the method comprises combining the Bcl inhibitor with a means for inhibiting Mcl-1.

5. A method of enhancing the senolytic activity of an Mcl-1 inhibitor and/or the therapeutic efficacy of the Mcl-1 inhibitor for treating a senescence associated disease or disorder, wherein the method comprises combining the Mcl-1 inhibitor with a means for inhibiting Bcl.

6. The method of claims 1-5, wherein the senescence-associated disease or disorder is not cancer.

7. The method of claims 1-6, wherein the Bcl inhibitor is a Bcl-2/Bcl-xL/Bcl-w inhibitor, a Bcl-2/Bcl-xL inhibitor, a Bcl-xL/Bcl-w inhibitor, or a Bcl-xL selective inhibitor.

8. The method of claims 1-7, wherein the Bcl inhibitor is any one of the Bcl inhibitors listed or exemplified in this disclosure.

9. The method of claims 1-8, wherein the Mcl-1 inhibitor is a small molecule compound, a peptide mimetic, a BH3-derived peptide, or a stapled peptide.

10. The method of claims 1-9, wherein the Mcl-1 inhibitor is any one of the Mcl-1 inhibitors listed or exemplified in this disclosure.

11. The method of claims 1-10, wherein the Bcl inhibitor is navitoclax (ABT263) and the Mcl-1 inhibitor is selected from AMG-176, AZD-5991, S-63845, and A1210477.

12. The method of claims 1-10, wherein the Bcl inhibitor is (R)-5-(4-chlorophenyl)-4-(3-fluoro-5-(4-(4-(4-(4-(4-(hydroxymethyl)piperidin-1-yl)-1-(phenylthio)butan-2-ylamino)-3-(trifluoromethylsulfonyl)phenylsulfonamido)phenyl)piperazin-1-yl)phenyl)-1-isopropyl-2-methyl-1H-pyrrole-3-carboxylic acid (Compound 26) and the Mcl-1 inhibitor is selected from AMG-176, AZD-5991, and S-63845.

13. The method of claims 1-10, wherein the Bcl inhibitor is A-1331852 and the Mcl-1 inhibitor is AMG-176.

14. The method of claims 1-13, wherein the Bcl inhibitor and the Mcl-1 inhibitor in combination have a synergy coefficient (δ) greater than 10 for killing irradiated small airway epithelial cells (SAEC).

15. The method of claim 14, wherein the synergy coefficient (δ) is between 10-100.

16. The method of claims 1-15, wherein the senescent cells are senescent endothelial cells, senescent fibroblasts, senescent mesenchymal cells, senescent chondrocytes, or senescent synoviocytes.

17. The method of claims 1-15, wherein the cells are senescent epithelial cells.

18. The method of claims 1-16, wherein the senescence-associated disease or disorder is atherosclerosis.

19. The method of claims 1-16, wherein the senescence-associated disease or disorder is osteoarthritis.

20. The method of claims 1-16, wherein the senescence-associated disease or disorder is a pulmonary disease, such as idiopathic pulmonary fibrosis (IPF) or chronic obstructive pulmonary disease (COPD).

21. The method of claims 1-16, wherein the senescence-associated disease or disorder is an eye disease or disorder, such as age-related macular degeneration, glaucoma, or diabetic retinopathy.

22. The method of claims 1-16, wherein the senescence-associated disease or disorder is a liver disease, such as non-alcoholic steatohepatitis (NASH), primary biliary cholangitis (PBC), or primary sclerosing cholangitis (PSC).

23. The method of claims 1-2, 4-22, wherein the Bcl inhibitor and the Mcl-1 inhibitor are administered as a combination within at least one treatment cycle, which treatment cycle comprises a treatment course followed by a non-treatment interval; and wherein the total dose of the combination administered during the treatment cycle is an amount less than the amount effective for a cancer treatment.

24. The method of claim 3, wherein the senolytic combination contacts the senescent cell within at least one treatment cycle, which treatment cycle comprises a treatment course followed by a non-treatment interval; and wherein the total dose of the senolytic combination administered during the treatment cycle is an amount less than the amount effective for a cancer treatment.

25. The method of claims 1-17, 19-21, 23, wherein the Bcl inhibitor and the Mcl-1 inhibitor are administered directly to an organ or tissue affected by the senescence-associated disease or disorder that comprises the senescent cells.

26. The method of claims 18, 22-23, wherein the Bcl inhibitor and the Mcl-1 inhibitor are administered systemically.

27. The method of claim 2 or 22, wherein the senolytic combination is administered directly to an organ or tissue affected by the senescence-associated disease or disorder that comprises the senescent cells.

28. The method of claim 3, 18, 22, 24, wherein the senolytic combination is administered systemically.

29. A combination of a Bcl inhibitor medicament and an Mcl-1 inhibitor medicament for treating a senescence-associated disease or disorder, wherein the Bcl inhibitor medicament and the Mcl-1 inhibitor medicament selectively kill senescent cells.

30. Use of a Bcl inhibitor in combination with an Mcl-1 inhibitor for the manufacture of a medicament for the treatment of a senescence-associated disease or disorder in a subject.

31. A unit dose of a pharmaceutical composition that is formulated for relief of symptoms of a senescence-associated disease or disorder at a disease site in a subject in need thereof;
wherein the unit dose contains an amount of a first compound that constitutes a means for selectively inhibiting Bcl and an amount of a second compound that constitutes a means for specifically inhibiting Mcl-1 in a formulation that is configured for administration in or around the site of the disorder in the subject the subject;
wherein the formulation of the composition, the amount of the first compound, the amount of the second compound, and the molar ratio of the first compound to the second compound configure the unit dose such that one or more administrations of the unit dose in or around the disease site during a treatment period is effective in selectively removing senescent cells from the disease site, and
thereby providing the subject with a subsequent therapeutic period during which the signs or symptoms of the senescence-associated disease or disorder are relieved as a result of the administration of the composition to the disease site during the treatment period.

32. A method of identifying a combination of medicaments that is effective for killing senescent cells or treating a senescence-associated disease or disorder, the method comprising:
(1) contacting a senescent cell (such as an irradiated cell) with a predetermined concentration and molar ratio of a test Bcl inhibitor and a test Mcl-1 inhibitor;
(2) contacting a non-senescent cell (such as a non-irradiated cell of the same tissue type) with the same concentration and molar ratio of the test Bcl inhibitor and the test Mcl-1 inhibitor; and
(3) identifying the test Bcl inhibitor and the test Mcl-1 inhibitor as an effective combination of medicaments at said concentration and molar ratio if the combination has an LD50 that is selective (such as 3, 5, or 10 times lower) for the senescent cells compared with the non-senescent cells.


SEQUENCE LISTING (3 sequences)

SEQ ID NO: 1 - DNA, 19 bp, Artificial Sequence
  Synthetic Sequence
ctgcccaacgcaccgaata

SEQ ID NO: 2 - DNA, 20 bp, Artificial Sequence
  Synthetic Sequence
gctgcccatcatcatgacct

SEQ ID NO: 3 - DNA, 20 bp, Artificial Sequence
  Synthetic Sequence
ttacggtcggaggccgatcc